(19) United States
(12) Patent Application Publication
(10) Pub. No.: US 2011/0021473 A1
(43) Pub. Date: Jan. 27, 2011

(54) INHIBITORS OF PHOSPHODIESTERASE TYPE-IV

(75) Inventors: Sonali Rudra, Gurgaon (IN); Nidhi Gupta, New Delhi (IN); Lalit Kumar Baregama, Kapasan (IN); Ritu Agarwal, West Bengal (IN); Vinayak Vasantrao Khairnar, Nashik (IN); Saswati Chakladar, New Delhi (IN); Mandadapu Raghu Ramaiah, Andra Pradesh (IN); Nagarajan Muthukamal, Tamil Nadu (IN); Sarala Balachandran, Mumbai (IN); Sarika Ramnani, Uttar Pradesh (IN); Venkata P. Palle, Aundh Pune (IN); Sunanda G. Dastidar, New Delhi (IN); Abhijit Ray, New Delhi (IN); Lalitha Vijaykrishan, New Delhi (IN); Jitendra Sattigeri, Gurgaon (IN)

Correspondence Address: Ranbaxy Inc., Intellectual Property Department, 600 College Road East, PRINCETON, NJ, 08540, US

(73) Assignee: Ranbaxy Laboratories Limited

(21) Appl. No.: 12/442,257

(22) PCT Filed: Sep. 22, 2007
(86) PCT No.: PCT/IB2007/053854

§ 371 Date: Jan. 6, 2010

(30) Foreign Application Priority Data
Sep. 22, 2006 (IN) 2097/DEL/2006

(51) Int. Cl. A61K 31/424; C07D 498/10; C07D 261/20; A61K 31/423; A61K 31/4439; A61K 31/5377; A61K 31/454; A61K 31/573; A61P 29/00; A61P 25/00; A61P 17/00; A61P 11/00; A61P 19/00; A61P 31/18

(52) U.S. Cl. 514/171; 548/240; 514/378; 546/15; 514/278; 544/70; 514/236.8

(57) ABSTRACT

The present invention relates to catechol derivatives of formula (I), which can be used as inhibitors of phosphodiesterase (PDPI) type 4 or type 7, Compounds disclosed herein can be useful in the treatment of CNS disorders, inflammatory diseases such as, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans. Processes for the preparation of disclosed compounds are provided, as well as pharmaceutical compositions containing the disclosed compounds, and their use as phosphodiesterase (PDE) type 4 or type 7 inhibitors.

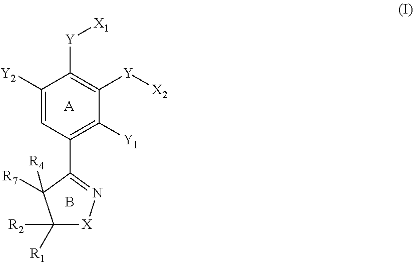

### FIELD OF THE INVENTION

The present invention relates to catechol derivatives, which can be used as inhibitors of phosphodiesterase (PDE) type 4 or type 7.

Compounds disclosed herein can be useful in the treatment of CNS disorders, inflammatory diseases such as, AIDS, asthma, arthritis; bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

Processes for the preparation of disclosed compounds are provided, as well as pharmaceutical compositions containing the disclosed compounds, and their use as phosphodiesterase (PDE) type 4 or type 7 inhibitors.

### BACKGROUND OF THE INVENTION

It is known that cyclic adenosine-3′,5′-monophosphate (cAMP) exhibits an important role of acting as an intracellular secondary messenger (Sutherland and Roll, Pharmacol. Rev (1960);12:265). Its intracellular hydrolysis to adenosine 5′-monophosphate (AMP) causes number of inflammatory conditions which are not limited to psoriasis, allergic rhinitis, shock, atopic dermatitis, crohn's disease, adult respiratory distress syndrome (ARDS), eosinophilic granuloma, allergic conjunctivitis, osteoarthritis, ulcerative colitis. The most important role in the control of cAMP (as well as of cGMP) levels is played by cyclic nucleotide phosphodiesterases (PDE) which represent a biochemically and functionally, highly variable superfamily of the enzyme. Eleven distinct families of phosphodiesterases with more than 2.5 gene products are currently recognized. Although PDE1, PDE2, PDE4, and PDE7 all use cAMP as a substrate, only the PDE4 and PDE7 types are highly selective for hydrolysis of cAMP. Inhibitors of PDE, particularly the PDE4 inhibitors, such as rolipram or Ro-1724 are therefore known as cAMP-enhancers. Immune cells contain type 4 and type 3 PDE, the PDE4 type being prevalent in human mononuclear cells. Thus the inhibition of phosphodiesterase type 4 has been a target for modulation and, accordingly, for therapeutic intervention in a range of disease processes. Studies have shown that administering PDE4 inhibitors can have a restorative effect on memory loss in animal models, including those of Alzheimer's disease (Expert Opin. Ther. Targets (2005) 9 (6): 1283-1305; Drug Discovery Today, (2005); 10 (22): 1503-19).

The potential importance of subtypes of PDE4 in terms of development of new inhibitors of PDE4 has recently emerged. In PDE4B-deficient mice, but not those lacking PDE4D, there is a profound reduction in the ability of LPS to generate TNFα from stimulated peripheral blood leukocytes (Jin and Conti, Proc Natl Acad Sci (2002); 99 (11): 7628-33). It would appear that development of more specific PDE4B inhibitors may be useful, since the PDE4B knock-out mice showed reduced duration of xylazine/ketamine-triggered anaesthesia which is used as a surrogate marker for emesis in mice, which do not usually demonstrate vomiting (Robichaud et al., 2002, J. Clin. Invest. 110:1045).

Of the other cAMP family of proteins discovered so far, PDE7A also offers itself as a promising candidate for inhibitor development because of its cellular distribution in almost all pro inflammatory and immune cells (Curr Pharm Des. (2006); 12 (25): 3207-20). Additionally, it has been shown to be a prime modulator of human T cell function (Science. (1999) February 5; 283 (5403): 848-51).

The initial observation that xanthine derivatives, theophylline and caffeine inhibit the hydrolysis of cAMP led to the discovery of the required hydrolytic activity in the cyclic nucleotide phosphodiesterase (PDE) enzymes. More recently, distinct classes of PDEs have been recognized (Beavo and Reifsnyder, Trends Pharmacol. Sci., (1990); 11: 150), and their selective inhibition has led to improved drug therapy Nichoius, Challiss and Shahid, Trends Pharmacol. Sci., (1991); 12: 19). Thus it was recognized that inhibition of PDE 4 could lead to inhibition of inflammatory mediator release (Verghese et. al, J. Mol. Cell. Cardiol., (1989), 12 (Suppl.II): S 61).

WO 2004046095 discloses certain arylthiourea derivatives and related compounds, which possess antiviral activity. WO 00/35891 discloses certain morpholinone and morpholine derivative, which are selective antagonists for human α₁ₐ receptor. WO 2004050024 discloses 3-aminopyrrolidine derivatives and their use as modulators of chemokine receptors. WO 2005/21515 relates to isoxazoline derivatives, which can be used as selective inhibitors of phosphodiesterase (PDE) type IV. WO2005/051931 discloses phosphodiesterase inhibitors.

### SUMMARY OF INVENTION

The present invention provides catechol derivatives, which can be used for the treatment of CNS disorders, inflammatory diseases such as, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans, and the processes for the synthesis of these compounds.

Pharmaceutically acceptable salts, pharmaceutically acceptable solvates, enantiomers, diastereomers, polymorphs or N-oxides of these compounds having the same type of activity are also provided.

Pharmaceutical compositions containing the compounds, which may also contain pharmaceutically acceptable carriers or diluents, can be used for CNS disorders, inflammatory diseases such as, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

The present invention encompasses a compound having the structure of Formula I,

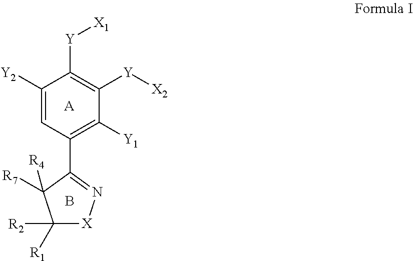

its pharmaceutically acceptable salts, pharmaceutically acceptable solvates, enantiomers, diastereomers, polymorphs or N-oxides wherein
when X is oxygen,
R₁ can be hydrogen, alkyl, heterocyclyl, —(CH₂)₁₋₄OR′, provided that R₂ is also (CH₂)₁₋₄OR′ (wherein R′ can be hydrogen, alkyl, alkenyl, alkynyl, (un)saturated cycloalkyl, aryl, heterocyclyl or heteroaryl), —C(═O)NRₓR_{y} provided that R₂ is also —C(═O)NRₓR_{y} [wherein Rₓ and R_{y} can be hydrogen, alkyl, alkenyl of three to six carbon atoms, alkynyl of three to six carbon atoms, cycloalkyl, —SO₂R₅ (wherein R₅ can be hydrogen, alkyl, alkenyl, alkynyl, aryl, cycloalkyl, alkaryl, heteroaryl, heteroarylalkyl, heterocyclyl or heterocyclylalkyl), aryl, alkaryl, heteroaryl, heterocyclyl, heteroarylalkyl, and heterocyclylalkyl], —(CH₂)ₘ—C(═O)R₃ (wherein in can be an integer in the range of 0-2 and R₃ can be cycloalkyl, aryl, optionally substituted Rₚ or R_{q}, wherein Rₚ can be heterocyclyl or heteroaryl ring wherein the said rings can be attached to (CH₂)ₘC(═O) through N, and R_{q} can be heterocyclyl or heteroaryl ring wherein the said rings can be attached to —(CH₂)ₘC(═O) through C);
R₂ can be —(CH₂)ₘC(═O)R₃ (wherein m and R₃ are the same as defined earlier), —(CH₂)₁₋₄OR′, provided R₁ is also (CH₂)₁₋₄OR′ (wherein R′ is same as defined earlier); —C(═O)NRₓR_{y} provided R₁ is also —C(═O)NRₓR_{y} (wherein Rₓ and R_{y} are same as defined earlier), or R₁ and R₂ may together form optionally substituted cycloalkyl or heterocyclyl ring wherein the substituents of such a joint R₁-R₂ ring(s) can be oxo, alkyl, alkenyl, alkynyl, halogen (F, Cl, Br, I), nitro, —NH₂, ═NOH, —C(═O)NRₓR_{y}), —COORₓ, —COONRₓR_{y} (wherein Rₓ and R_{y} are the same as defined earlier), —NHCOOR₆ (wherein R₁₅ can be alkyl, alkenyl, alkynyl, cycloalkyl, alkaryl, heteroarylalkyl or heterocyclylalkyl), cyano, hydroxy, alkoxy, or substituted amino;
R₄ can be hydrogen, alkyl, —OR₅ (wherein R₅ is the same as defined earlier), halogen (F, Cl, Br, I), —NH₂, substituted amino, cyano, carboxy, or —C(═O)NRₓR_{y} (wherein Rₓ and R_{y} are the same as defined above), or R₂ and R₄ may together form optionally substituted 4-12 membered (un)saturated monocyclic or bicyclic ring system fused to ring B having 0-4 heteroatom(s) selected from N, O and S with the proviso that R₂ and R₄ together does not form —CH₂—O—CH₂—O—CH₂—, wherein the substituents can be one or more of alkyl, halogen (F, Cl, Br, I), hydroxy, alkoxy, —NH₂ or substituted amino;
R₇ can be hydrogen, alkyl, alkenyl, alkynyl, —OR₅ (wherein R₅ is the same as defined earlier), halogen (F, Cl, Br, I), cyano, —NH₂ or substituted amino;
X₁ and X₂ can be hydrogen, alkyl, alkaryl, cycloalkyl, heterocyclyl, heteroaryl, aryl, heteroarylalkyl or heterocyclylalkyl, —(CH₂)ₘCOR₃, —(CH₂)_{g}C(═O)NRₓR_{y} or —(CH₂)_{g1}C(═O)OR₃ (wherein g and g₁ can be an integer from 0-3, m, R_{y} and R₃ are the same as defined earlier);
Y can be an oxygen atom, a sulphur atom, or —NR (wherein R can be hydrogen, acyl, aryl, or alkyl);
Y₁ and Y₂ can be independently selected from hydrogen, alkyl, —OR (wherein R is the same as defined earlier), —SR (wherein R is the same as defined earlier, and —NHR (wherein R is the same as defined earlier);
Any of Y₁ and X₂ & X₁ and Y₂ may together form a cyclic ring fused with the ring A shown in Formula I, the ring containing 3-5 carbon atoms within the ring and having 1-3 heteroatoms such as N, O and S.
X₁ and X₂ may together form a cyclic ring fused with the ring A shown in Formula I, the ring containing 3-5 carbon atoms within the ring and having 2-3 heteroatoms such as N, O and S.
When X is NR₇ or S, wherein R₇ is hydrogen or lower alkyl (C₁-C₆)
R₁ and R₂ can be independently alkyl, alkenyl, alkynyl, alkoxy, hydroxyl, cyano, nitro, halogen (F, Cl, Br, I), heteroaryl, heterocyclyl, heteroarylalkyl, heterocyclylalkyl, —NH₂, substituted amino, carboxy, —(CH₂)ₘ(C═O)R₃ (wherein m and R₃ are the same as defined earlier), —C(═O)NRₓR_{y} (wherein Rₓ and R_{y} are the same as defined above), or —(CH₂)₁₋₄OR′ (wherein R′ is same as defined earlier) or R₁ and R₂ may together form optionally substituted cycloalkyl or heterocyclyl ring wherein the substituents of such a joint R₁-R₂ ring(s) can be oxo, alkyl, alkenyl, alkynyl, halogen (F, Cl, Br, I), nitro, —NH₂, ═NOH, —C(═O)NRₓR_{y}, —COORₓ, —COONRₓR_{y}, (wherein Rₓ and R_{y} are the same as defined earlier), —NHCOOR₆ (wherein R₆ can be alkyl, alkenyl, alkynyl, cycloalkyl, alkaryl, heteroarylalkyl or heterocyclylalkyl), cyano, hydroxy, alkoxy or substituted amino;
R₄ can be hydrogen, alkyl, halogen (F, Cl, Br, I), —OR₅ (wherein R₅ is the same as defined earlier), cyano, carboxy, —NH₂, substituted amino, or —C(═O)NRₓR_{y} (wherein Rₓ and R_{y} are the same as defined above), or R₂ and R₄ may together form optionally substituted 4-12 membered (un)saturated monocyclic or bicyclic ring system fused to ring B having 0-4 heteroatom(s) such as N, O and S, with the proviso that R₂ and R₄ together does not form —CH₂—O—CH₂—O—CH₂—, wherein the substituents can be one or more of alkyl, halogen (F, Cl, Br, I), hydroxy, alkoxy, or amino;
R₇ can be hydrogen, alkyl, alkenyl, alkynyl, —OR₅ (wherein R₅ is the same as defined earlier), halogen (F, Cl, Br, I), cyano, —NH₂ or substituted amino;
X₁ and X₂ can be alkyl, cycloalkyl, alkaryl, heteroaryl, heterocyclyl, heteroarylalkyl or heterocyclylalkyl, —(CH₂)_{g}C(═O)NRₓR_{y} or —(CH₂)_{g1}C(═O)OR₃ (wherein g and g₁ can be an integer from 0-3, R₃, Rₓ and R_{y} are the same as defined earlier);
Y can be an oxygen atom, a sulphur atom, or —NR (wherein R can be hydrogen, acyl, aryl or alkyl);
Y₁ and Y₂ can be independently hydrogen, alkyl, —OR (wherein R is the same as defined earlier), —SR (wherein R is the same as defined earlier), or —NHR (wherein R is the same as defined earlier);
Any of Y₁ and X₂ & X₁ and Y₂ may together form a cyclic ring fused with the ring A shown in Formula I, the ring containing 3-5 carbon atoms within the ring and having 1-3 heteroatoms such as N, O and S;
X₁ and X₂ may together form a cyclic ring fused with the ring A shown in Formula I, the ring containing 3-5 carbon atoms within the ring and having 2-3 heteroatoms such as N, O and S.
The following definitions apply to terms as used herein.

The term “alkyl,” unless otherwise specified, refers to a monoradical branched or unbranched saturated hydrocarbon having from 1 to about 20 carbon atoms. This term is exemplified by groups such as methyl, ethyl, n-propyl, iso-propyl, n-butyl, iso-butyl, t-butyl, n-hexyl, n-decyl, tetradecyl, and the like. The alkyl groups may further be substituted with one or more substituents such as alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryloxy, aminosulfonyl, aminocarbonylamino, hydroxyamino, alkoxyamino, nitro, —S(O)ₙR₅ (wherein n is 0, 1 or 2 and R₅ is the same as defined earlier), heterocyclyl or heteroaryl. Unless otherwise constrained by the definition, all substituents may optionally be further substituted by 1-3 substituents chosen from alkyl, carboxy, aminocarbonyl, hydroxy, alkoxy, halogen, —CF₃, amino, substituted amino, cyano, and —S(O)ₙR₅ (wherein R₅ and n are same as defined earlier) or an alkyl group as defined above that is interrupted by 1-5 atoms or groups independently chosen from oxygen, sulfur and —NRₐ— (where Rₐ is chosen from hydrogen, alkyl, cycloalkyl, alkenyl, alkynyl, aryl). Unless otherwise constrained by the definition, all substituents may optionally be further substituted by 1-3 substituents chosen from alkyl, carboxy, aminocarbonyl, hydroxy, alkoxy, halogen, CF₃, amino, substituted amino, cyano, and —S(O)ₙR₅ (wherein n and R₅ are the same as defined earlier); or an alkyl group as defined above that has both substituents as defined above and is also interrupted by 1-5 atoms or groups as defined above.

The term “alkenyl” unless otherwise specified, refers to a monoradical of a branched or unhranched unsaturated hydrocarbon group preferably having from 2 to 20 carbon atoms with cis or trans geometry. Preferred alkenyl groups include ethenyl or vinyl (CH═CH₂), 1-propylene or allyl(—CH₂CH═CH₂), iso-propylene (—C(CH₃)═CH₂), bicyclo[2.2.1]heptene, and the like. In the event that alkenyl is attached to a heteroatom, the double bond cannot be alpha to the heteroatom. The alkenyl group may further be substituted with one or more substituents such as alkyl, alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryl, aryloxy, aminosulfonyl, aminocarbonylamino, hydroxyamino, alkoxyamino, nitro, —S(O)ₙR₅ (wherein n and R₅ are the same as defined earlier), heterocyclyl or heteroaryl. Unless otherwise constrained by the definition, all substituents may optionally be further substituted by 1-3 substituents chosen from alkyl, carboxy, aminocarbonyl, hydroxy, alkoxy, halogen, —CF₃, amino, substituted amino, cyano, and —S(O)ₙR₅ (wherein R₅ and n are the same as defined earlier).

The term “alkynyl” unless otherwise specified, refers to a monoradical of an unsaturated hydrocarbon, preferably having from 2 to 20 carbon atoms. Preferred alkynyl groups include ethynyl (—C═CH), propargyl (or propynyl; —CH₂C═CH), and the like.

In the event that alkynyl is attached to a heteroatom, the triple bond cannot be alpha to the heteroatom. The alkynyl group may further be substituted with one or more substituents such as alkyl, alkenyl, alkynyl, alkoxy, cycloalkyl, acyl, acylamino, acyloxy, amino, aminocarbonyl, alkoxycarbonylamino, azido, cyano, halogen, hydroxy, OXO, thiocarbonyl, carboxy, arylthio, thiol, alkylthio, aryl, aryloxy, aminosulfonyl, aminocarbonylamino, hydroxyamino, alkoxyamino, nitro, —S(O)ₙR₅ (wherein R₅ and n are the same as defined earlier). Unless otherwise constrained by the definition, all substituents may optionally be further substituted by 1-3 substituents chosen from alkyl, carboxy, aminocarbonyl, hydroxy, alkoxy, halogen, CF₃, amino, substituted amino, cyano, and —S(O)ₙR₅ (wherein R₅ and n are the same as defined earlier).

The term “cycloalkyl” refers to saturated or unsaturated cyclic alkyl groups of from 3 to 20 carbon atoms having a single cyclic ring or multiple condensed rings, which contains an optional olefinic bond. Such cycloalkyl groups include, by way of example, single ring structures such as cyclopropyl, cyclobutyl, cyclopentyl, cyclohexyl, cyclooctyl, cyclopropylene, cyclobutylene and the like, or multiple ring structures such as adamantanyl, and bicyclo[2.2.1]heptane, 1,4-dioxa-spiro[4,5]decane or cyclic alkyl groups to which is fused an aryl group, thr example indane, and the like. The cycloalkyl may further be substituted with one or more substituents such as alkyl, alkenyl, alkynyl, alkoxy, cycloalkyl, acyl, acylamino, acyloxy, amino, aminocarbonyl, alkoxycarbonylamino, azido, cyano, halogen, hydroxy, oxo, thiocarbonyl, carboxy, arylthio, thiol, alkylthio, aryl, aryloxy, aminosulfonyl, aminocarbonylamino, hydroxyamino, alkoxyamino, nitro, —S(O)ₙR₅ (wherein R₅ and n are the same as defined earlier), heteroaryl or heterocyclyl. Unless otherwise constrained by the definition, all substituents may optionally be further substituted by 1-3 substituents chosen from alkyl, carboxy, aminocarbonyl, hydroxy, alkoxy, halogen, CF₃, —NH₂, substituted amino, cyano, and —S(O)ₙR₅ (wherein R₅ and n are the same as defined earlier).

The term “alkoxy” denotes the group O-alkyl, wherein alkyl is the same as defined above.

The term “alkaryl” refers to alkyl-aryl linked through alkyl (wherein alkyl is the same as defined earlier) portion and the said alkyl portion contains carbon atoms from 1-6 and aryl is same as defined below.

The term “aryl” herein refers to phenyl, naphthyl, 2,3-dihydro-1H-indenyl or indanyl ring and the like optionally substituted with 1 to 3 substituents selected from the group consisting of halogen (F, Cl, Br, I), hydroxy, alkyl, alkenyl, alkynyl, cycloalkyl, alkoxy, aryloxy, oxo, —S(O)ₙRₛ (wherein R₅ is the same as defined earlier), cyano, nitro, carboxy, heterocyclyl, heteroaryl, heterocyclylalkyl, amino, —NHCOalkyl, —NHCOOalkyl, —NHSO₂alkyl, heteroarylalkyl, acyl or (CH₂)₀₋₃C(═O)NRₓR_{y} (wherein Rₓ and R_{y} are same as defined earlier).

The term “carboxy” as defined herein refers to —C(═O)O—R₆ wherein R₆ can be for example, hydrogen, alkyl, alkenyl, alkynyl, cycloalkyl, alkaryl, heteroarylalkyl or heterocyclylalkyl.

The term “heteroaryl” unless otherwise specified, refers to an aromatic ring structure containing 5 or 6 carbon atoms, or a bicyclic aromatic group having 8 to 10 carbon atoms, with one or more heteroatom(s) independently selected from the group consisting of N, O and S, optionally substituted with 1 to 3 substituent(s) such as halogen (F, Cl, Br, I), hydroxy, alkyl, alkenyl, alkynyl, cycloalkyl, aryl, —S(O)ₙR₅ (wherein n and R₅ are the same as defined earlier), alkoxy, alkaryl, cyano, nitro, acyl or C(═O)NRₓR_{y} (wherein Rₓ and R_{y} are the same as defined earlier). Examples of heteroaryl groups are pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like such as analogous oxygen, sulphur, and mixed hetero atom containing groups.

The term ‘heterocyclyl” unless otherwise specified refers to a saturated or unsaturated monocyclic or polycyclic ring having 3 to 10 atoms, in which 1 to 3 carbon atoms in a ring are replaced by heteroatoms selected from the group comprising of O, S, SO, SO₂, N or N-oxide, and are optionally benzofused or fused heteroaryl of 5-6 ring members and are optionally substituted wherein the substituents can be halogen (F, Cl, Br, I), hydroxy, alkyl, alkenyl, alkynyl, hydroxyalkyl, cycloalkyl, carboxy, aryl, alkoxy, alkaryl, heteroaryl, heterocyclyl, heteroarylalkyl, heterocyclylalkyl, oxo, alkoxyalkyl or —S(O)ₙR₅ (wherein n and R₅ are the same as defined earlier), cyano, nitro, —NH₂, substituted amino, acyl or —C(═O)NRₓR_{y} (wherein Rₓ and R_{y} are the same as defined earlier). Examples of heterocyclyl groups are tetrahydrofuranyl, dihydrofuranyl, azabicyclohexane, dihydropyridinyl, piperidinyl, isoxazoline, piperazinyl, dihydrobenzofuryl, morpholinyl, pyrrolidinyl, oxetane, tetrahydropyranyl, thietane, tetrahydrothiophene-1-oxide, tetrahydrothiophene, isoindole-dione, dihydroindolyl,

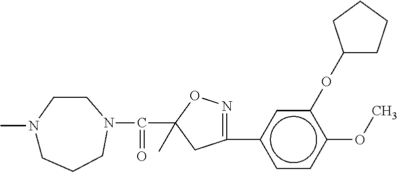

and the like.

“Heteroarylalkyl” refers to alkyl-heteroaryl group, wherein the alkyl and heteroaryl are the same as defined earlier.

“Heterocyclylalkyl” refers to alkyl-heterocyclyl group, wherein the alkyl and heterocyclyl are the same as defined earlier.

The term “acyl” as defined herein refers to —C(═O)R″ wherein R″ is the hydrogen, alkyl, alkaryl, cycloalkyl, aryl, heterocyclyl, heteroaryl, heteroarylalkyl or heterocyclylalkyl.

“Substituted amino” unless and otherwise specified refers to a group —N(Rₖ)₂ wherein each Rₖ is independently selected from the group consisting of hydrogen [provided that both Rₖ groups are not hydrogen (defined as “—NR₂”)], alkyl, alkenyl, alkynyl, alkaryl, cycloalkyl, aryl, heteroaryl, heterocyclyl, heterocyclylalkyl, heteroarylalkyl, acyl, —S(O)ₘR₅ wherein m and R₅ is the same as defined above, —C(═O)NRₓR_{y}, —C(═O)ORₓ (wherein Rₓ and R_{y} are the same as defined earlier) or NHC(═O)NR_{y}Rₓ (wherein R_{y} and Rₓ are the same as defined earlier).

Unless otherwise constrained by the definition, all substituents may optionally be further substituted by 1-3 substituents chosen from alkyl, alkaryl, cycloalkyl, aryl, heteroaryl, heterocyclyl, carboxy, hydroxy, alkoxy, halogen, —CF₃, cyano, —C(═O)NRₓR_{y}, —O(CO)NRₓR_{y} (wherein Rₓ and R_{y} are the same as defined earlier) and —OC(═O)NRₓR_{y}, —S(O)ₘR₅ (where R₅ is the same as defined above and m is 0, 1 or 2).

The compounds of the present invention can be used for treating CNS disorders, inflammatory diseases such as, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases especially in humans.

In accordance with yet another aspect, there are provided processes for the preparation of the compounds as described herein.

### DETAILED DESCRIPTION OF THE INVENTION

The compounds of the present invention may be prepared by techniques well known in the art. In addition, the compounds of the present invention may be prepared following a reaction sequence as depicted below.

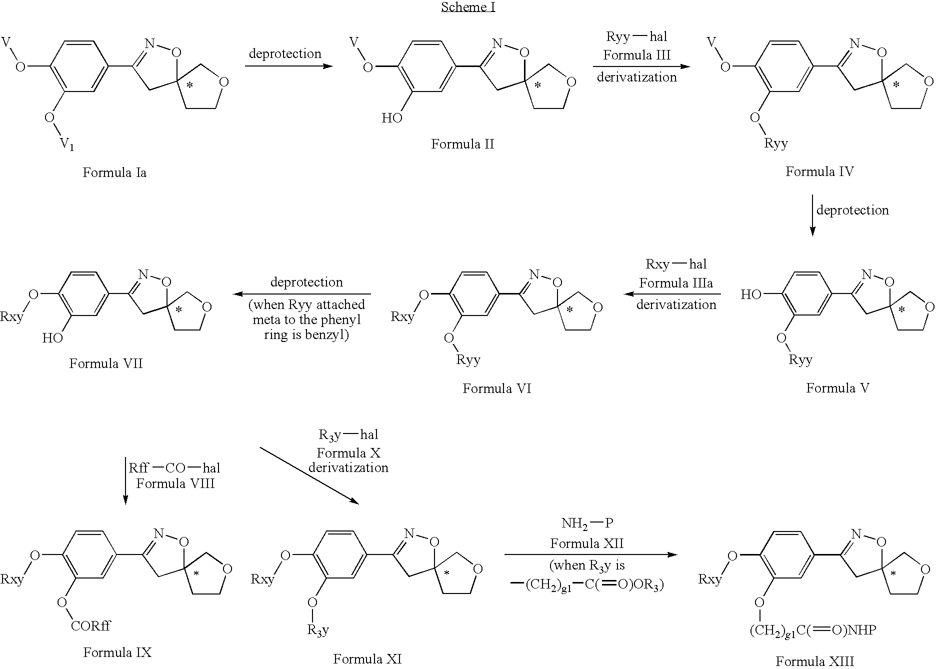

The compounds of Formulae II, IV, V, VI, VII, TX, XI and XIII can be prepared by following the procedure as depicted in Scheme I. The reaction comprises deprotecting a compound of Formula Ia [wherein * refers to chiral centre (racemic or R or S isomer); V is alkyl and V₁ is cycloalkyl] to give a compound of Formula II, which can be reacted with a compound of Formula III (wherein hal is Br, Cl or I; Ryy can be alkyl, aryl, cycloalkyl, alkaryl, heteroaryl, heterocyclyl, heteroarylalkyl, heterocyclylalkyl, —(CH₂)_{g1}COOR₃, —(CH₂)ₘCOR₃ or —(CH₂)_{g}C(═O)NRₓR_{y}) (wherein R₃, g, m, Rₓ, R_{y} and g₁ are the same as defined earlier) to give a compound of Formula IV, which can be deprotected to give a compound of Formula V, which can be reacted with a compound of Formula IIIa to give a compound of Formula VI (wherein hal is Br, Cl or I; Rxy can be alkyl, cycloalkyl, alkaryl, heteroaryl, heterocyclyl, heteroarylalkyl, heterocyclylalkyl), which can be deprotected (when Ryy attached meta to the phenyl ring is benzyl) to give a compound of Formula VII, which can be reacted with a compound of Formula VIII (wherein Rff can be alkyl, cycloalkyl, alkaryl, aryl, heteroaryl, heterocyclyl, heteroarylalkyl or heterocyclylalkyl and hal is Br, Cl or I)) to give a compound of Formula IX. The compound of Formula VII can be reacted with a compound of Formula X (wherein R_{3y} can be —(CH₂)_{g1}C(═O)OR₃, —(CH₂)ₘCOR₃, alkyl, cycloalkyl, alkaryl, heteroaryl, heterocyclyl, heteroarylalkyl, heterocyclylalkyl or —(CH₂)_{g}C(═O)NRₓR_{y}) to give a compound of Formula XI, which can be reacted with a compound of Formula XII (wherein P is selected from alkyl, aralkyl, cycloalkyl, —C(═O)Oaralkyl, —C(═O)OC(CH₃)₃, —C(═O)OC(CH₃)₂CHBr₂ or C(═O)OC(CH₃)₂CCl₃) to give a compound of Formula XIII.

The deprotection of a compound of Formula Ia to give a compound of Formula II can be carried out in an organic solvent selected from dichloromethane, dichloroethane, chloroform or carbon tetrachloride in the presence of Lewis acid as a catalyst selected from aluminium trichloride, aluminium tribromide, zirconium tetrachloride, tin chloride or trichlorobismuthine.

The reaction of a compound of Formula II with a compound of Formula III to give a compound of Formula IV can be carried out in an organic solvent selected from dimethylformamide, tetrahydrofuran, diethylether or dioxane in the presence of a base selected from potassium carbonate, sodium carbonate or sodium bicarbonate.

The deprotection of a compound of Formula IV to give a compound of Formula V can be carried with an agent selected from sodiumethane thiolate, sodium decane thiolate, sodium dodecane thiolate, sodium thiocresolate in an organic solvent selected from N,N-dimethylacetamide, hexamethyl phosphoramide or dimethylformamide.

The reaction of a compound of Formula V with a compound of Formula IIIa to give a compound of Formula VI can be carried out in an organic solvent selected from dimethylformamide, tetrahydrofuran, diethylether or dioxane in the presence of a base selected from potassium carbonate, sodium carbonate or sodium bicarbonate.

The deprotection of a compound of Formula VI to give a compound of Formula VII can be carried out in an organic solvent selected from methanol, ethanol, propanol or isopropylalcohol in the presence of palladium on carbon, palladium on carbon with ammonium formate or palladium hydroxide.

The reaction of a compound of VII with a compound of Formula VIII to give a compound of Formula IX can be carried out in an organic solvent selected from dimethylformamide, tetrahydrofuran, diethylether or dioxane in the presence of a base selected from sodium hydride, potassium hydride, triethyl amine, potassium carbonate or sodium bicarbonate.

The reaction of a compound of Formula VII with a compound of Formula X to give a compound of Formula XI can be carried out in an organic solvent selected from dimethylformamide, tetrahydrofuran, diethylether or dioxane in the presence of a base selected from potassium carbonate, sodium carbonate or sodium bicarbonate.

The compound of Formula XI can be reacted with a compound of Formula XII to give a compound of Formula XIII

Particular compounds are listed below:
3-[2-(Difluoromethoxy)-5-(1,7-dioxa-2-azaspiro[4.4]non-2-en-3-yl)phenoxy]prop (Compound No. 2);
[2-(Difluoromethoxy)-5-(1,7-dioxa-2-azaspiro[4.4]non-2-en-3-yl)phenoxy]acetonitrile (Compound No. 3);
4-[(5S or 5R)-1,7-Dioxa-2-azaspiro[4.4]non-2-en-3-yl]-2-methoxyphenol (Compound No. 4);
4-[(5R or 5S)-1,7-Dioxa-2-azaspiro[4.4]non-2-en-3-yl]-2-methoxyphenol (Compound No. 5);
5-[(5S or 5R)-1,7-Dioxa-2-azaspiro[4.4]non-2-en-3-yl]-2-methoxyphenol (Compound No. 6);
(5S or 5R)-3-(3,4-Dimethoxyphenyl)-1,7-dioxa-2-azaspiro[4.4]non-2-ene (Compound No. 7);
(5R or 5S)-3-(3,4-Dimethoxyphenyl)-1,7-dioxa-2-azaspiro[4.4]non-2-ene (Compound No. 8);
2-(Benzyloxy)-4-(1,7-dioxa-2-azaspiro[4.4]non-2-en-3-yl)phenol (Compound No. 9)
2-[2-(Difluoromethoxy)-5-(1,7-dioxa-2-azaspiro[4.4]non-2-en-3-yl)phenoxy]ethanol (Compound No. 10);
3-[4-(Difluoromethoxy)-3-ethoxyphenyl)-1,7-dioxa-2-azaspiro[4.4]non-2-ene (Compound No. 11);
3-(3-(Cyclohexyloxy)-4-(difluoromethoxy)phenyl]-1,7-dioxa-2-azaspiro[4.4]non-2-ene (Compound No. 12);
(5R or 5S)-3-[4-(Difluoromethoxy)-3-methoxyphenyl]-1,7-dioxa-2-azaspiro[4.4]non-2-ene (Compound No. 13);
(5S or 5R)-3-[4-(Difluoromethoxy)-3-methoxyphenyl]-1,7-dioxa-2-azaspiro[4.4]non-2-ene (Compound No. 14);
Ethyl [2-(difluoromethoxy)-5-(1,7-dioxa-2-azaspiro[4.4]non-2-en-3-yl)phenoxy]acetate (Compound No. 15);
-2-(Cyclopropylmethoxy)-4-[(5R or 5S)-1,7-dioxa-2-azaspiro[4.4]non-2-en-3-yl]phenol (Compound No. 65);
4-[(5R or 5S)-1,7-Dioxa-2-azaspiro[4.4]non-2-en-3-yl]-2-isopropoxyphenol (Compound No. 66);
(5R or 5S)-3-[3-(Cyclopropylmethoxy)-4-(difluoromethoxy)phenyl]-1,7-dioxa-2-azaspiro[4.4]non-2-ene (Compound No. 67);
(5R or 5S)-3-[4-(difluoromethoxy)-3-isopropoxyphenyl]-1,7-dioxa-2-azaspiro[4.4]non-2-ene (Compound No. 68)
3-[4-(Difluoromethoxy)-3-(2-morpholin-4-ylethoxy)phenyl]-1,7-dioxa-2-azaspiro[4.4]non-2-ene (Compound No. 16);
2-(Difluoromethoxy)-5-(1,7-dioxa-2-azaspiro[4.4]non-2-en-3-yl)phenyl cyclohexanecarboxylate (Compound No. 17);
5-[2-(Difluoromethoxy)-5-(1,7-dioxa-2-azaspiro[4.4]non-2-en-3-yl)phenoxy]pentanoic acid (Compound No. 18);
3-[3-(2,2,2-Trifluoroethoxy)-4-(difluoromethoxy)phenyl]-1,7-dioxa-2-azaspiro[4.4]non-2-ene (Compound No. 19);
3-[3-(Cyclopentylmethoxy)-4-(difluoromethoxy)phenyl]-1,7-dioxa-2-azaspiro[4.4]non-2-ene (Compound No. 20);
N-cyclopropyl-2-[2-(difluoromethoxy)-5-(1,7-dioxa-2-azaspiro[4.4]non-2-en-3-yl)phenoxy]acetamide (Compound No. 21);
2-[2-(Difluoromethoxy)-5-(1,7-dioxa-2-azaspiro[4.4]non-2-en-3-yl)phenoxy]acetamide (Compound No. 22);
2-[2-(Difluoromethoxy)-5-(1,7-dioxa-2-azaspiro[4.4]non-2-en-3-yl)phenoxy]-N-methylacetamide (Compound No. 23);
3-[3-(Cyclopentyloxy)-4-(2,2,2-trifluoroethoxy)phenyl]-1,7-dioxa-2-azaspiro[4.4]non-2-ene (Compound No. 24);
2-(Difluoromethoxy)-5-(1,7-dioxa-2-azaspiro[4.4]non-2-en-3-yl)phenyl cyclopropanecarboxylate (Compound No. 25);
2-(Difluoromethoxy)-5-(1,7-dioxa-2-azaspiro[4.4]non-2-en-3-yl)phenyl morpholine-4-carboxylate (Compound No. 26);
2-(Difluoromethoxy)-5-(1,7-dioxa-2-azaspiro[4.4]non-2-en-3-yl)phenyl benzoate (Compound No. 27);
5-[2-(Difluoromethoxy)-5-(1,7-dioxa-2-azaspiro[4.4]non-2-en-3-yl)phenoxy]pentanamide (Compound No. 28);
3-[3-Propoxy-4-(2,2,2-trifluoroethoxy)phenyl]-1,7-dioxa-2-azaspiro[4.4]non-2-ene (Compound No. 29);
3-[3-Isopropoxy-4-(2,2,2-trifluoroethoxy)phenyl]-1,7-dioxa-2-azaspiro[4.4]non-2-ene (Compound No. 30);
3-[3-(Cyclopropylmethoxy)-4-(2,2,2-trifluoroethoxy)phenyl]-1,7-dioxa-2-azaspiro[4.4]non-2-ene (Compound No. 31);
3-[3-(2,3-Dihydro-1H-inden-2-yloxy)-4-(2,2,2-trifluoroethoxy)phenyl]-1,7-dioxa-2-azaspiro[4.4]non-2-ene (Compound No. 32);
5-(1,7-Dioxa-2-azaspiro[4.4]non-2-en-3-yl)-2-(2,2,2-trifluoroethoxy)phenol (Compound No. 33);
3-[3-Methoxy-4-(2,2,2-trifluoroethoxy)phenyl]-1,7-dioxa-2-azaspiro[4.4]non-2-ene (Compound No. 34);
3-[3-Ethoxy-4-(2,2,2-trifluoroethoxy)phenyl]-1,7-dioxa-2-azaspiro[4.4]non-2-ene (Compound No. 35);
3-[3-Butoxy-4-(2,2,2-trifluoroethoxy)phenyl]-1,7-dioxa-2-azaspiro[4.4]non-2-ene (Compound No. 36);
3-[3-(Cyclohexylmethoxy)-4-(2,2,2-trifluoroethoxy)phenyl]-1,7-dioxa-2-azaspiro[4.4]non-2-ene (Compound No. 37);
3-{[2-(Difluoromethoxy)-5-(1,7-dioxa-2-azaspiro[4.4]non-2-en-3-yl)phenoxy]ethyl}benzonitrile (Compound No. 38);
2-{2-[2-(difluoromethoxy)-5-(1,7-dioxa-2-azaspiro[4.4]non-2-en-3-yl)phenoxy]ethyl}-1H-isoindole-1,3(2H)-dione (Compound No. 39);
3-[3-(Cyclohexyloxy)-4-(2,2,2-trifluoroethoxy)phenyl]-1,7-dioxa-2-azaspiro[4.4]non-2-ene (Compound No. 40);
Ethyl [5-(1,7-dioxa-2-azaspiro[4.4]non-2-en-3-yl)-2-(2,2,2-trifluoroethoxy)phenoxy]acetate (Compound No. 41);
3-[3-(Cyclohexylmethoxy)-4-(difluoromethoxy)phenyl]-1,7-dioxa-2-azaspiro[4.4]non-2-ene (Compound No. 42);
Tert-butyl [2-(difluoromethoxy)-5-(1,7-dioxa-2-azaspiro[4.4]non-2-en-3-yl)phenoxy]acetate (Compound No. 43);
N-cyclopropyl-2-[5-(1,7-dioxa-2-azaspiro[4.4]non-2-en-3-yl)-2-(2,2,2-trifluoroethoxy)phenoxy]acetamide (Compound No. 44);
N-benzyl-2-[5-(1,7-dioxa-2-azaspiro[4.4]non-2-en-3-yl)-2-(2,2,2-trifluoroethoxy)phenoxy]acetamide (Compound No. 47);
N-Cyclopentyl-2-[5-(1,7-dioxa-2-azaspiro[4.4]non-2-en-3-yl)-2-(2,2,2-trifluoroethoxy)phenoxy]acetamide (Compound No. 48);
(5S or 5R)-3-(3-isopropoxy-4-methoxyphenyl)-1,7-dioxa-2-azaspiro[4.4]non-2-ene (Compound No. 57);
(5S or 5R)-3-[3-(Cyclopropylmethoxy)-4-methoxyphenyl]-1,7-dioxa-2-azaspiro[4.4]non-2-ene (Compound No. 58);
2-(Cyclopropylmethoxy)-4-[(5S or 5R)-1,7-dioxa-2-azaspiro[4.4]non-2-en-3-yl]phenol (Compound No. 59);
4-[(5S or 5R)-1,7-Dioxa-2-azaspiro[4.4]non-2-en-3-yl]-2-isopropoxyphenol (Compound No. 60);
(5S or 5R)-3-[3-(Cyclopropylmethoxy)-4-(difluoromethoxy)phenyl]-1,7-dioxa-2-azaspiro[4.4]non-2-ene (Compound No. 62);
(5S or 5R)-3-[4-(difluoromethoxy)-3-isopropoxyphenyl]-1,7-dioxa-2-azaspiro[4.4]non-2-ene (Compound No. 63);
(5R or 5S)-3-[4-(difluoromethoxy)-3-isopropoxyphenyl]-1,7-dioxa-2-azaspiro[4.4]non-2-ene (Compound No. 64);
(5S or 5R)-3-[3-(Benzyloxy)-4-(difluoromethoxy)phenyl]-1,7-dioxa-2-azaspiro[4.4]non-2-ene (Compound No. 76);
2-(Benzyloxy)-4-[(5S or 5R)-1,7-dioxa-2-azaspiro[4.4]non-2-en-3-yl]phenol (Compound No. 77);
(5S or 5R)-3-[3-(Benzyloxy)-4-methoxyphenyl]-1,7-dioxa-2-azaspiro[4.4]non-2-ene (Compound No. 78);
3-[3-(Benzyloxy)-4-(2,2,2-trifluoroethoxy)phenyl]-1,7-dioxa-2-azaspiro[4.4]non-2-ene (Compound No. 90);
2-(Difluoromethoxy)-5-[(5S or 5R)-1,7-dioxa-2-azaspiro[4.4]non-2-en-3-yl]phenol (Compound No. 91);
5-[(5R or 5S)-1,7-Dioxa-2-azaspiro[4.4]non-2-en-3-yl)-2-methoxyphenol (Compound No. 92);
(5R or 5S)-3-(3-(benzyloxy)-4-methoxyphenyl]-1,7-dioxa-2-azaspiro[4.4]non-2-ene (Compound No. 126);
2-(Benzyloxy)-4-[(5R or 5S)-1,7-dioxa-2-azaspiro[4.4]non-2-en-3-yl]phenol (Compound No. 127);
(5R or 5S)-3-[3-(Benzyloxy)-4-(difluoromethoxy)phenyl]-1,7-dioxa-2-azaspiro[4.4]non-2-ene (Compound No. 128);
2-(Difluoromethoxy)-5-[(5R or 5S)-1,7-dioxa-2-azaspiro[4.4]non-2-en-3-yl]phenol (Compound No. 130);
   pharmaceutically acceptable salts, pharmaceutically acceptable solvates, enantiomers, diastereomers, polymorphs or N-oxides thereof.

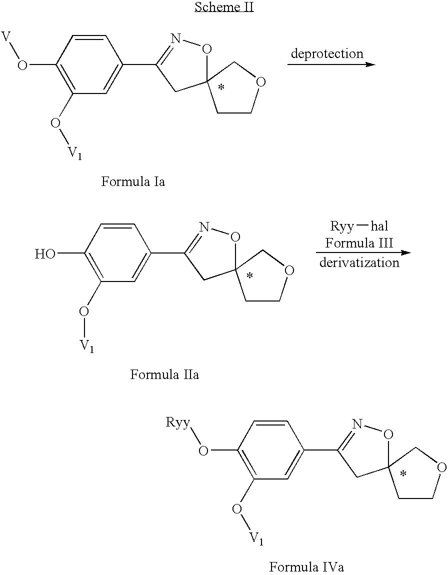

The compounds of Formulae IIa and IVa can be prepared by following the procedure as depicted in Scheme II. Thus the reaction comprises deprotecting a compound of Formula Ia (wherein V and V₁ are the same as defined earlier) [wherein * represents chiral centre (racemic or optically active)] to give a compound of Formula IIa which can be reacted with a compound of Formula III (wherein hal is Br, Cl or I; Ryy can be alkyl, aryl, cycloalkyl, alkaryl, heteroaryl, heterocyclyl, heteroarylalkyl or heterocyclylalkyl, —(CH₂)_{g}C(═O)NRₓR_{y}, —(CH₂)ₘCOR₃ or —(CH₂)_{g1}C(═O)OR₃ (wherein m, R₃, g, g₁, Rₓ and R_{y} are the same as defined earlier) to give a compound of Formula IVa.

The deprotection of a compound of Formula Ia to give a compound of Formula IIa can be carried out with an agent selected from sodiumethane thiolate, sodium decane thiolate, sodium dodecane thiolate, sodium thiocresolate in an organic solvent selected from N,N-dimethylacetamide, hexamethyl phosphoramide or dimethylformamide.

The reaction of a compound of Formula IIa with a compound of Formula III to give a compound of Formula IVa can be carried out in an organic solvent selected from dimethylformamide, tetrahydrofuran, diethylether or dioxane in the presence of a base selected from potassium carbonate, sodium carbonate or sodium bicarbonate.

Particular compounds are described below:
2-(Cyclopentyloxy)-4-[(5R or 5S)-1,7-dioxa-2-azaspiro[4.4]non-2-en-3-yl]phenol (Compound No. 45);
2-(Cyclopentyloxy)-4-[(5S or 5R)-1,7-dioxa-2-azaspiro[4.4]non-2-en-3-yl]phenol (Compound No. 46);
(5R or 5S)-3-[3-(cyclopentyloxy)-4-(difluoromethoxy)phenyl]-1,7-dioxa-2-azaspiro[4.4]non-2-ene (Compound No. 56);
(5S or 5R)-3-[3-(cyclopentyloxy)-4-(difluoromethoxy)phenyl]-1,7-dioxa-2-azaspiro[4.4]non-2-ene (Compound No. 61);
   pharmaceutically acceptable salts, pharmaceutically acceptable solvates, enantiomers, diastereomers, polymorphs or N-oxides thereof.

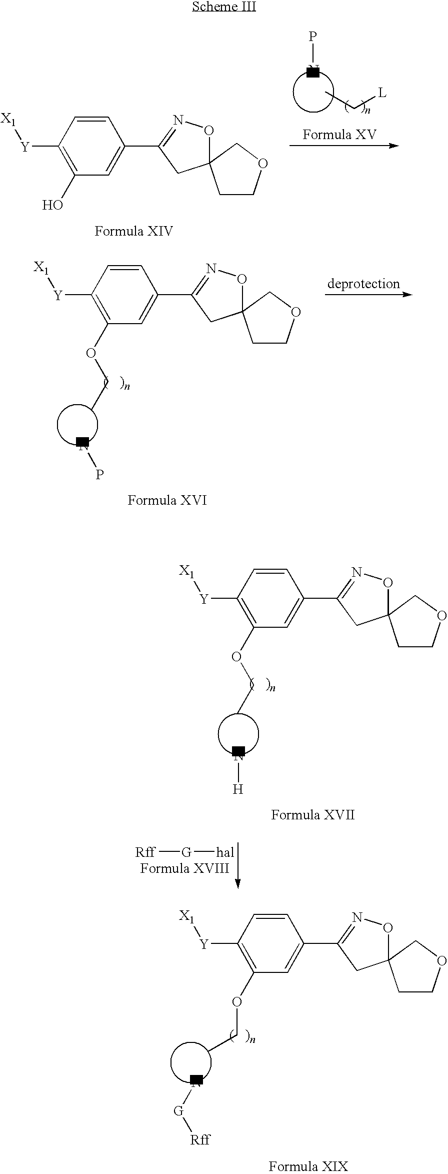

The compounds of Formula XVII and XIX can be prepared by following the procedure as depicted in Scheme III. Thus the reaction comprises reacting a compound of Formula XIV (wherein X₁ and Y are the same as defined earlier) with a compound of Formula XV (wherein P is the same as defined earlier and L is a leaving group selected from hal (Br, Cl or I), —Omesyl, —Otosyl or —Otriflyl) to give a compound of Formula XVI (wherein n is in integer from 0-2), which can be deprotected to give a compound of Formula XVII, which can be reacted with a compound of Formula XVIII (wherein G is —CO or —SO₂ and Rff is the same as defined earlier) to give a compound of Formula XIX.

The reaction of a compound of Formula XIV with a compound of Formula XV to give a compound of Formula XVI can be carried out in an organic solvent selected from dimethylformamide, tetrahydrofuran, diethylether or dioxane in the presence of a base selected from potassium carbonate, sodium carbonate or sodium bicarbonate.

The deprotection of a compound of Formula XVI (when P is —C(═O)OC(CH₃)₃ or —C(═O)OC(CH₃)₇CHBr₂) to give a compound of Formula XVII can be carried out in, for example, hydrochloric acid solution of methanol, ethanol, propanol, isopropylalcohol, tetrahydrofuran or ether.

Alternatively, the deprotection of a compound of Formula XVI (when P is —C(═O)OC(CH₃)₃ or —C(═O)OC(CH₃)₂CHBr₂) to give a compound of Formula XVII can be carried with trifluoroacetic acid in dichloromethane.

The deprotection of a compound of Formula XVI (when P is —C(═O)OC(CH₃)₂CCl₃) to give a compound of Formula XVII can be carried out by a supernucleophile (for example, lithium cobalt (I) phthalocyanine, zinc and acetic acid or cobalt phthalocyanine).

The deprotection of a compound of Formula XVI (when P is aralkyl) to give a compound of Formula XVII can be carried out in an organic solvent selected from methanol, ethanol, propanol or isopropylalcohol in the presence of palladium on carbon in presence of hydrogen gas or palladium on carbon with a source of hydrogen gas selected from ammonium formate solution, cyclohexene or formic acid).

The reaction of a compound of Formula XVII with a compound of Formula XVIII to give a compound of Formula XIX can be carried out in an organic solvent selected from dimethylformamide, tetrahydrofuran, diethylether or dioxane in the presence of a base selected from potassium carbonate, sodium carbonate or sodium bicarbonate.

Particular compounds are described below:
3-[3-{[(3S)-1-Benzylpyrrolidin-3-yl]oxy}-4-(difluoromethoxy)phenyl]-1,7-dioxa-2-azaspiro[4.4]non-2-ene (Compound No. 1);
Tert-butyl 4-[2-(difluoromethoxy)-5-(1,7-dioxa-2-azaspiro[4.4]non-2-en-3-yl)phenoxy]piperidine-1-carboxylate (Compound No. 49);
Hydrochloride salt of 3-[4-(difluoromethoxy)-3-(piperidin-4-yloxy)phenyl]-1,7-dioxa-2-azaspiro[4.4]non-2-ene (Compound No. 50);
3-{3-[(1-Acetylpiperidin-4-yl)oxy]-4-(difluoromethoxy)phenyl}-1,7-dioxa-2-azaspiro[4.4]non-2-ene (Compound No. 51);
Tert-butyl (3S)-3-[2-(difluoromethoxy)-5-(1,7-dioxa-2-azaspiro[4.4]non-2-en-3-yl)phenoxy]pyrrolidine-1-carboxylate (Compound No. 52);
Tert-butyl (3R)-3-[2-(difluoromethoxy)-5-(1,7-dioxa-2-azaspiro[4.4]non-2-en-3-yl)phenoxy]pyrrolidine-1-carboxylate (Compound No. 53);
Tert-butyl 3-[2-(difluoromethoxy)-5-(1,7-dioxa-2-azaspiro[4.4]non-2-en-3-yl)phenoxy]piperidine-1-carboxylate (Compound No. 54);
Tert-butyl (2S)-2-{[2-(difluoromethoxy)-5-(1,7-dioxa-2-azaspiro[4.4]non-2-en-3-yl)phenoxy]methyl}pyrrolidine-1-carboxylate (Compound No. 55);
Hydrochloride salt of 3-{4-(Difluoromethoxy)-3-[(3S)-pyrrolidin-3-yloxy]phenyl}-1,7-dioxa-2-azaspiro[4.4]non-2-ene (Compound No. 69);
Hydrochloride salt of 3-{4-(difluoromethoxy)-3-[(2S)-pyrrolidin-2-ylmethoxy]phenyl}-1,7-dioxa-2-azaspiro[4.4]non-2-ene (Compound No. 70);
Hydrochloride salt of 3-{4-(difluoromethoxy)-3-[(2R)-pyrrolidin-2-ylmethoxy]phenyl}-1,7-dioxa-2-azaspiro[4.4]non-2-ene (Compound No. 71);
3-[4-(Difluoromethoxy)-3-{[(2R)-1-propionylpyrrolidin-2-yl]methoxy}phenyl]-1,7-dioxa-2-azaspiro[4.4]non-2-ene (Compound No. 72);
3-[3-{[(2S)-1-acetylpyrrolidin-2-yl]methoxy}-4-(difluoromethoxy)phenyl]-1,7-dioxa-2-azaspiro[4.4]non-2-ene (Compound No. 73);
3-[3-{[(3S)-1-benzoylpyrrolidin-3-yl]oxy}-4-(difluoromethoxy)phenyl]-1,7-dioxa-2-azaspiro[4.4]non-2-ene (Compound No. 74);
3-[4-(Difluoromethoxy)-3-{[(3S)-1-propionylpyrrolidin-3-yl]oxy}phenyl]-1,7-dioxa-2-azaspiro[4.4]non-2-ene (Compound No. 75);
3-{4-(Difluoromethoxy)-3-[(1-propionylpiperidin-4-yl)oxy]phenyl}-1,7-dioxa-2-azaspiro[4.4]non-2-ene (Compound No. 79);
3-[4-(Difluoromethoxy)-3-{[1-(4-fluorobenzoyl)piperidin-4-yl]oxy}phenyl]-1,7-dioxa-2-azaspiro[4.4]non-2-ene (Compound No. 80);
3-[3-{[1-(Cyclopropylcarbonyl)piperidin-4-yl]oxy}-4-(difluoromethoxy)phenyl]-1,7-dioxa-2-azaspiro[4.4]non-2-ene (Compound No. 81);
3-[3-{[1-(Cyclopentylcarbonyl)piperidin-4-yl]oxy}-4-(difluoromethoxy)phenyl]-1,7-dioxa-2-azaspiro[4.4]non-2-ene (Compound No. 82);
3-[4-(Difluoromethoxy)-3-({1-[(trifluoromethyl)sulfonyl]piperidin-4-yl}oxy)phenyl]-1,7-dioxa-2-azaspiro[4.4]non-2-ene (Compound No. 83);
3-{3-[(1-Acetylpiperidin-3-yl)oxy]-4-(difluoromethoxy)phenyl}-1,7-dioxa-2-azaspiro[4.4]non-2-ene (Compound No. 84);
3-{4-(Difluoromethoxy)-3-[(1-propionylpiperidin-3-yl)oxy]phenyl}-1,7-dioxa-2-azaspiro[4.4]non-2-ene (Compound No. 85);
3-[4-(Difluoromethoxy)-3-{[1-(4-fluorobenzoyl)piperidin-3-yl]oxy}phenyl]-1,7-dioxa-2-azaspiro[4.4]non-2-ene (Compound No. 86);
3-[3-{[1-(Cyclopropylcarbonyl)piperidin-3-yl]oxy}-4-(difluoromethoxy)phenyl]-1,7-dioxa-2-azaspiro[4.4]non-2-ene (Compound No. 87);
3-[3-{[1-(Cyclopentylcarbonyl)piperidin-3-yl]oxy}-4-(difluoromethoxy)phenyl]-1,7-dioxa-2-azaspiro[4.4]non-2-ene (Compound No. 88);
3-[4-(Difluoromethoxy)-3-{[1-(ethylsulfonyl)piperidin-3-yl]oxy}phenyl]-1,7-dioxa-2-azaspiro[4.4]non-2-ene (Compound No. 89);
3-[3-{[(3S)-1-acetylpyrrolidin-3-yl]oxy}-4-(difluoromethoxy)phenyl]-1,7-dioxa-2-azaspiro[4.4]non-2-ene (Compound No. 93);
Hydrochloride salt of 3-[4-(Difluoromethoxy)-3-(piperidin-3-yloxy)phenyl]1,7-dioxa-2-azaspiro[4.4]non-2-ene (Compound No. 94);
3-[4-(Difluoromethoxy)-3-{[1-(phenylcarbonyl)piperidin-4-yl]oxy}phenyl]-1,7-dioxa-2-azaspiro[4.4]non-2-ene (Compound No. 95);
3-[4-(Difluoromethoxy)-3-{[1-(morpholin-4-ylcarbonyl)piperidin-4-yl]oxy}phenyl]-1,7-dioxa-2-azaspiro[4.4]non-2-ene (Compound No. 96);
3-[4-(1-Difluoromethoxy)-3-{[1-(phenylcarbonyl)piperidin-3-yl]oxy}phenyl]-7-dioxa-2-azaspiro[4.4]non-2-ene (Compound No. 97);
3-[4-(Difluoromethoxy)-3-{[1-(morpholin-4-ylcarbonyl)piperidin-3-yl]oxy}phenyl]-1,7-dioxa-2-azaspiro[4.4]non-2-ene (Compound No. 98);
3-[4-(Difluoromethoxy)-3-({1-[(trifluoromethyl)sulfonyl]piperidin-3-yl}oxy)phenyl]-1,7-dioxa-2-azaspiro[4.4]non-2-ene (Compound No. 99);
3-[4-(Difluoromethoxy)-3-{[(2R)-1-(phenylcarbonyl)pyrrolidin-2-yl]methoxy}phenyl]-1,7-dioxa-2-azaspiro[4.4]non-2-ene (Compound No. 100);
3-[3-{[(2R)-1-acetylpyrrolidin-2-yl]methoxy}-4-(difluoromethoxy)phenyl]-1,7-dioxa-2-azaspiro[4.4]non-2-ene (Compound No. 101);
3-[4-(Difluoromethoxy)-3-{[(2R)-1-propanoylpyrrolidin-2-yl]methoxy}phenyl]-1,7-dioxa-2-azaspiro[4.4]non-2-ene (Compound No. 102);
3-{[3-[(2R)-1-(cyclopropylcarbonyl)pyrrolidin-2-yl]methoxy}-4-(difluoromethoxy)phenyl]-1,7-dioxa-2-azaspiro[4.4]non-2-ene (Compound No. 103);
3-[3-{[(3S)-1-(cyclopropylcarbonyl)pyrrolidin-3-yl]oxy}-4-(difluoromethoxy)phenyl]-1,7-dioxa-2-azaspiro[4.4]non-2-ene (Compound No. 104);
3-[3-{[(3S)-1-(cyclopentylcarbonyl)pyrrolidin-3-yl]oxy}-4-(difluoromethoxy)phenyl]-1,7-dioxa-2-azaspiro[4.4]non-2-ene (Compound No. 105);
3-[4-(Difluoromethoxy)-3-({(3R)-1-[(4-fluorophenyl)carbonyl]pyrrolidin-3-yl}oxy)phenyl]-1,7-dioxa-2-azaspiro[4.4]non-2-ene (Compound No. 106);
   pharmaceutically acceptable salts, pharmaceutically acceptable solvates, enantiomers, diastereomers, polymorphs or N-oxides thereof.

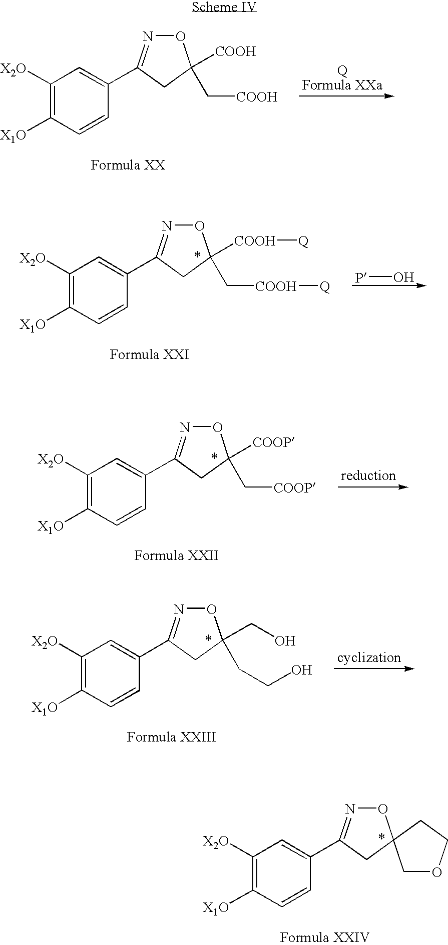

The compounds of Formula XXIV can be prepared by following the procedure as depicted in Scheme IV. Thus a compound of Formula XX (wherein X₁ and X₂ are the same as defined earlier) can be reacted with a compound of Formula XXa (wherein Q is a chiral resolving agent selected from IL-Ephedrine, D-Ephedrine, (+)-Brussian, (−)-Brussian, (1S,2R) (−)-cis-1-amino-2-indanol, (1R 2S) (+)-cis-4-amino-2-indanol, (1R,2R)-(−)-1,2-diamino cyclohexane or (1S,2S)-(+)-1,2-diamino cyclohexane, α-methylbenzylamine or β-methylbenzylamine) to give a compound of Formula XXI [wherein * refers to chiral centre (racemic or optically active)], which undergoes protection with a compound of Formula P′—OH to give a compound of Formula XXII (wherein P′ can be alkyl or aralkyl), which undergoes reduction to give a compound of Formula XXIII, which undergoes cyclisation to give a compound of Formula XXIV

The compound of Formula XX can be reacted with a compound of Formula XXa to give a compound of Formula XXI in an organic solvent, for example, acetone, ethanol, isopropyl alcohol, methanol, acetonitrile, tert-butyl alcohol, ethyl acetate, dioxane, dichloromethane or chloroform.

The protection of a compound of Formula XXI with a compound of Formula P′—OH to give a compound of Formula XXII can be carried out with halogenating agents, for example, thionyl chloride, oxalyl chloride, phosphorous pentachloride or phosphorous trichloride.

The compound of Formula XXII undergoes reduction to give a compound of Formula XXIII in an organic solvent, for example, tetrahydrofuran, dimethylformamide, diethyl ether or dioxane with a reducing agent selected from lithium aluminium hydride, sodium borohydride, borane dimethyl sulphide or lithium borohydride.

Alternatively, the compound of Formula XXIII can also be prepared by reducing free acid form of compound of Formula XXII.

The compound of Formula XXIII undergoes cyclisation to give a compound of Formula XXIV in an organic solvent, for example, tetrahydrofuran, dimethylformamide, dioxane or diethyl ether in the presence of a redox couple. The oxidizing part of the redox couple is selected from the group consisting of diisopropylazodicarboxylate (DIAD), diethylazodicarboxylate (DEAD), N,N,N′,N′-tetramethylazodicarboxylate (TMAD), 1,1′-(azodicarbonyl)dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′,N,′-tetraisopropylazodicarboxamide (TIPA). The reduction part of the redox couple is phosphine selected from the group consisting of trialkylphosphine (such as tributylphosphine), triarylphosphine (for example, triphenylphosphine), tricycloalkylphosphine (for example, triscyclohexylphosphine) or tetraheteroarylphosphine. The phosphine reagents with a combination of aryl, alkyl or heteroaryl substituents may also be used (for example, diphenylpyridylphosphine).

Particular compounds, which can be prepared following scheme IV, are disclosed below:
4-[(5S or 5R)-1,7-dioxa-2-azaspiro[4.4]non-2-en-3-yl]-2-methoxyphenol (Compound No. 4);
4-[(5R or 5S)-1,7-Dioxa-2-azaspiro[4.4]non-2-en-3-yl]-2-methoxyphenol (Compound No. 5);
5-[(5S or 5R)-1,7-Dioxa-2-azaspiro[4.4]non-2-en-3-yl]-2-methoxyphenol (Compound No. 6);
(5S or 5R)-3-(3,4-Dimethoxyphenyl)-1,7-dioxa-2-azaspiro[4.4]non-2-ene (Compound No. 7);
(5R or 5S)-3-(3,4-Dimethoxyphenyl)-1,7-dioxa-2-azaspiro[4.4]non-2-ene (Compound No. 8);
(5R or 5S)-3-[4-(Difluoromethoxy)-3-methoxyphenyl]-1,7-dioxa-2-azaspiro[4.4]non-2-ene (Compound No. 13);
(5S or 5R)-3-[4-(Difluoromethoxy)-3-methoxyphenyl]-1,7-dioxa-2-azaspiro[4.4]non-2-ene (Compound No. 14);
(5R or 5S)-3-[3-(cyclopentyloxy)-4-(difluoromethoxy)phenyl]-1,7-dioxa-2-azaspiro[4.4]non-2-ene (Compound No. 56);
(5S or 5R)-3-(3-isopropoxy-4-methoxyphenyl)-1,7-dioxa-2-azaspiro[4.4]non-2-ene (Compound No. 57);
(5S or 5R)-3-[3-(Cyclopropylmethoxy)-4-methoxyphenyl]-1,7-dioxa-2-azaspiro[4.4]non-2-ene (Compound No. 58);
2-(Cyclopropylmethoxy)-4-[(5S or 5R)-1,7-dioxa-2-azaspiro[4.4]non-2-en-3-yl]phenol (Compound No. 59);
4-[(5S or 5R)-1,7-Dioxa-2-azaspiro[4.4]non-2-en-3-yl]-2-isopropoxyphenol (Compound No. 60);
(5S or 5R)-3-[3-(cyclopentyloxy)-4-(difluoromethoxy)phenyl]-1,7-dioxa-2-azaspiro[4.4]non-2-ene (Compound No. 61);
(5S or 5R)-3-[4-(difluoromethoxy)-3-isopropoxyphenyl]-1,7-dioxa-2-azaspiro[4.4]non-2-ene (Compound No. 63);
(5R or 5S)-3-[4-(difluoromethoxy)-3-isopropoxyphenyl]-1,7-dioxa-2-azaspiro[4.4]non-2-ene (Compound No. 64);
2-(Cyclopropylmethoxy)-4-[(5R or 5S)-1,7-dioxa-2-azaspiro[4.4]non-2-en-3-yl]phenol (Compound No. 65);
4-[(5R or 5S)-1,7-Dioxa-2-azaspiro[4.4]non-2-en-3-yl]-2-isopropoxyphenol (Compound No. 66);
(5R or 5S)-3-[3-(Cyclopropylmethoxy)-4-(difluoromethoxy)phenyl]-1,7-dioxa-2-azaspiro[4.4]non-2-ene (Compound No. 67);
(5R or 5S)-3-[4-(difluoromethoxy)-3-isopropoxyphenyl]-1,7-dioxa-2-azaspiro[4.4]non-2-ene (Compound No. 68);
(5S or 5R)-3-[3-(Benzyloxy)-4-(difluoromethoxy)phenyl]-1,7-dioxa-2-azaspiro[4.4]non-2-ene (Compound No. 76);
2-(Benzyloxy)-4-[(5S or 5R)-1,7-dioxa-2-azaspiro[4.4]non-2-en-3-yl]phenol (Compound No. 77);
(5S or 5R)-3-[3-(Benzyloxy)-4-methoxyphenyl]-1,7-dioxa-2-azaspiro[4.4]non-2-ene (Compound No. 78);
2-(Difluoromethoxy)-5-[(5S or 5R)-1,7-dioxa-2-azaspiro[4.4]non-2-en-3-yl]phenol (Compound No. 91);
5-[(5R or 5S)-1,7-Dioxa-2-azaspiro[4.4]non-2-en-3-yl]-2-methoxyphenol (Compound No. 92);
   pharmaceutically acceptable salts, pharmaceutically acceptable solvates, enantiomers, diastereomers, polymorphs or N-oxides thereof.

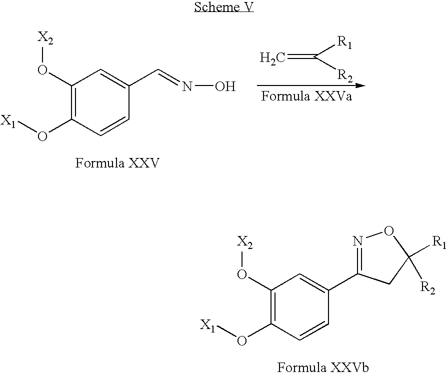

The compounds of Formula XXV b can be prepared by following the procedure as depicted in Scheme V. The reaction comprises reacting a compound of Formula XXV with a compound of Formula XXV a to give a compound of Formula XXV b.

The reaction of a compound of Formula XXV with a compound of Formula XXV a to give a compound of Formula XXV b can be carried out in the presence of one or more of reagents, for example, sodium hypochlorite, N-bromosuccinimide, N-chlorosuccinimide or mixtures thereof in an organic solvent, for example, tetrahydrofuran, dimethylformamide or dimethylsulphoxide.

Particular compounds prepared by this scheme are:
{3-[4-(Difluoromethoxy)-3-methoxyphenyl]-4,5-dihydroisoxazole-5,5-diyl}dimethanol (Compound No. 107);
3-[4-(Difluoromethoxy)-3-methoxyphenyl]-1,8-dioxa-2-azaspiro[4.5]dec-2-ene (Compound No. 111);
7-[4-(Difluoromethoxy)-3-methoxyphenyl]-5-oxa-6-azaspiro[3.4]oct-6-ene (Compound No. 115);
3-[4-(Difluoromethoxy)-3-methoxyphenyl]-1-oxa-2-azaspiro[4.4]non-2-ene (Compound No. 116);
3-[4-(Difluoromethoxy)-3-methoxyphenyl]-1-oxa-2-azaspiro[4.5]dec-2-ene (Compound No. 117);
   pharmaceutically acceptable salts, pharmaceutically acceptable solvates, enantiomers, diastereomers, polymorphs or N-oxides thereof.

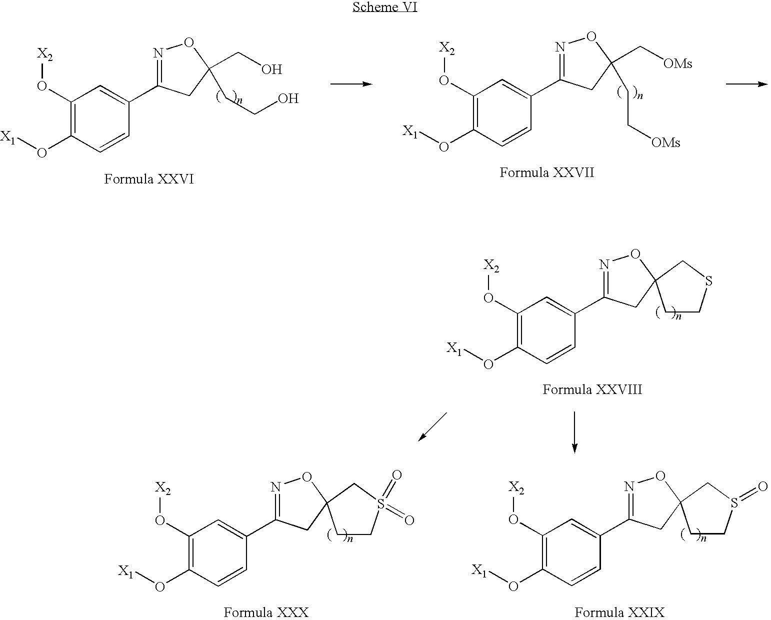

The compounds of Formulae XXVIII, XXIX and XXX can be prepared by following the procedure as depicted in Scheme VI. The reaction comprises the mesylation of a compound of Formula XXVI (wherein X₁ and X₂ are the same as defined earlier and n is an integer from 0-2) to give a compound of Formula XXVII, which can be cyclized to give a compound of Formula XXVIII, which can be oxidized to give compounds of Formulae XXIX and XXX.

The mesylation of a compound of Formula XXVI to give a compound of Formula XXVII can be carried out in the presence of one or more of mesylating agents, for example, methanesulfonyl chloride, methanesulfonic anhydride, trifluoromethanesulfonic anhydride, p-toluene sulphonyl chloride or mixtures thereof in the presence of one or more of bases, for example, triethylamine, pyridine, 2,6-lutidene, diisopropyl ethylamine or mixtures thereof in a solvent, for example, dichloromethane, chloroform, tetrahydrofuran or acetonitrile.

The cyclization of a compound of Formula XXVII to give a compound of Formula XXVIII can be carried out in the presence of one or more of hydrated or anhydrous alkali metal sulphides, for example, sodium sulphide in a solvent, for example, tetrahydrofuran, dimethylformamide, dimethylsulfoxide or dichloromethane.

The oxidation of a compound of Formula XXVIII to give compounds of Formulae XXIX and XXX can be carried out in the presence of one or more of oxidizing agents, for example, sodium periodate, m-chloroperoxybenzoic acid, text-butyl hydroperoxide or mixtures thereof in a solvent, for example, methanol, dichloromethane, tetrahydrofuran, dimethylformamide, dimethylsulfoxide, water or mixtures thereof.

Particular compounds prepared by this scheme are listed below:
3-[4-(Difluoromethoxy)-3-methoxyphenyl]-1-oxa-7-thia-2-azaspiro[4.4]non-2-ene (Compound No. 108);
3-[4-(Difluoromethoxy)-3-methoxyphenyl]-1-oxa-7-thia-2-azaspiro[4.4]non-2-ene 7-oxide (Compound No. 109);
7-[4-(Difluoromethoxy)-3-methoxyphenyl]-5-oxa-2-thia-6-azaspiro[3.4]oct-6-ene (Compound No. 110);
7-[4-(Difluoromethoxy)-3-methoxyphenyl]-5-oxa-2-thia-6-azaspiro[3.4]oct-6-ene 2-oxide (Compound No. 113);
3-[4-(Difluoromethoxy)-3-methoxyphenyl]-1-oxa-7-thia-2-azaspiro[4.4]non-2-ene 7,7-dioxide (Compound No. 114);
7-[4-(Difluoromethoxy)-3-methoxyphenyl]-5-oxa-2-thia-6-azaspiro[3.4]oct-6-ene 2,2-dioxide (Compound No. 120);
   pharmaceutically acceptable salts, pharmaceutically acceptable solvates, enantiomers, diastereomers, polymorphs or N-oxides thereof.

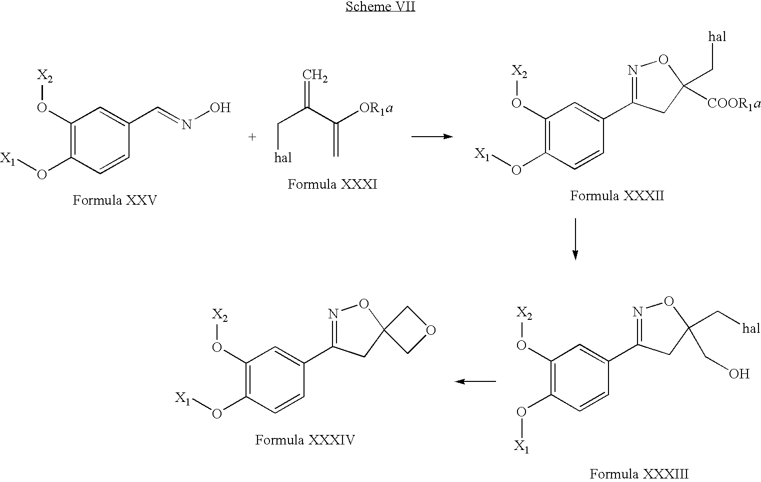

The compounds of Formula XXXIV can be prepared by following the procedure as depicted in Scheme VII. Accordingly, a compound of Formula XXV (wherein X₁ and X₂ are the same as defined earlier) can be reacted with a compound of Formula XXXI (wherein R₁ₐ can be alkyl and hal is the same as defined earlier) to give a compound of Formula XXXII, which can be reduced to give a compound of Formula XXXIII, which can be cyclized to give a compound of Formula XXXIV.

The reaction of a compound of Formula XXV with a compound of Formula XXXI to give a compound of Formula XXXII can be carried out, for example, by 1,3-dipolar cycloaddition reaction in the presence of one or more of reagents, for example, sodium hypochlorite, N-bromosuccinimide, N-chlorosuccinimide or mixtures thereof in a solvent, for example, dichloromethane, chloroform or mixtures thereof.

The reduction of a compound of Formula XXXII to give a compound of Formula XXXIII can be carried out in the presence of one or more of reducing agents, for example, sodium borohydride, lithium aluminium hydride, borane dimethyl sulphide or mixtures thereof in a solvent, for example, methanol, ethanol, tetrahydrofuran, ethyl acetate or mixtures thereof.

The cyclization of a compound of Formula XXXIII to give a compound of Formula XXXIV can be carried out in the presence of one or more of alkali metal hydroxides, for example, sodium hydroxide, potassium hydroxide or lithium hydroxide, alkali metal carbonates, for example, sodium carbonate or potassium carbonate, alkali metal alkoxides, for example, potassium t-butoxide, alkali metal hydrides, for example, sodium hydride or mixtures thereof in a solvent, for example, methanol, ethanol, tetrahydrofuran, dimethylformamide, water or mixtures thereof.

Particular compound prepared by this scheme is listed below:
7-[4-(Difluoromethoxy)-3-methoxyphenyl]-2,5-dioxa-6-azaspiro[3.4]oct-6-ene (Compound No. 123)
   pharmaceutically acceptable salts, pharmaceutically acceptable solvates, enantiomers, diastereomers, polymorphs or N-oxides thereof.

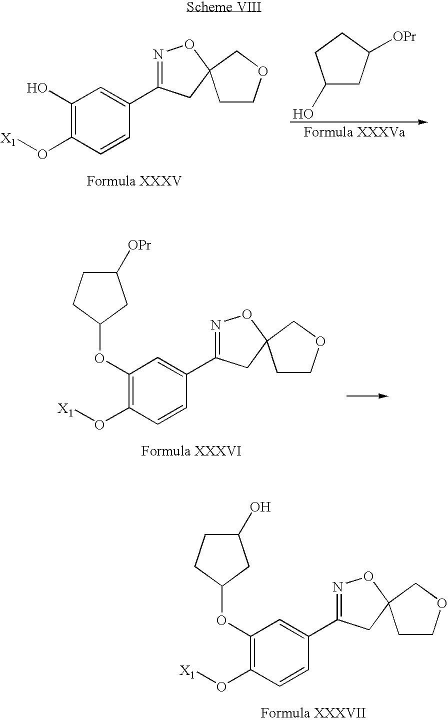

The compounds of Formula XXXVII can be prepared by following the procedure as depicted in Scheme VIII. Accordingly, a compound of Formula XXXV (wherein X₁ is same as defined earlier) can be reacted with a compound of Formula XXXV a to give a compound of Formula XXXVI (wherein Pr can be a protecting group, for example, tert-butyl dimethyl silyl) which can be deprotected to give a compound of Formula XXXVII.

The reaction of a compound of Formula XXXV with a compound of Formula XXXV a to give a compound of Formula XXXVI can be carried out in a solvent, for example, tetrahydrofuran, dimethylformamide, dimethoxy ethane, dioxane or diethyl ether in the presence of a redox couple. The oxidizing part of the redox couple is selected from the group consisting of diisopropyl azodicarboxylate (DIAD), diethylazodicarboxylate (DEAD), N,N,N′,N′-tetramethylazodicarboxylate (TMAD), 1,1′-(azodicarbonyl) dipiperidine (ADDP), cyanomethylenetributylphosphorane (CMBP), 4,7-dimethyl-3,5,7-hexahydro-1,2,4,7-tetrazocin-3,8-dione (DHTD) or N,N,N′,N,′-tetraisopropylazodicarboxamide (TIPA). The reduction part of the redox couple is phosphine selected from the group consisting of trialkylphosphine (such as tributylphosphine), triarylphosphine (for example, triphenylphosphine), tricycloalkylphosphine (for example, triscyclohexylphosphine) or tetraheteroarylphosphine. The phosphine reagents with a combination of aryl, alkyl or heteroaryl substituents may also be used (for example, diphenylpyridylphosphine).

The deprotection of a compound of Formula XXXVI to give a compound of Formula XXVIII can be carried out in a solvent, for example, methanol or ethanol in the presence of a acid, for example, hydrochloric acid.

Particular compound prepared by this scheme is listed below:
3-[2-(Difluoromethoxy)-5-(1,7-dioxa-2-azaspiro[4.4]non-2-en-3-yl)phenoxy]cyclopentanol (Compound No. 129)
   pharmaceutically acceptable salts, pharmaceutically acceptable solvates, enantiomers, diastereomers, polymorphs or N-oxides thereof.

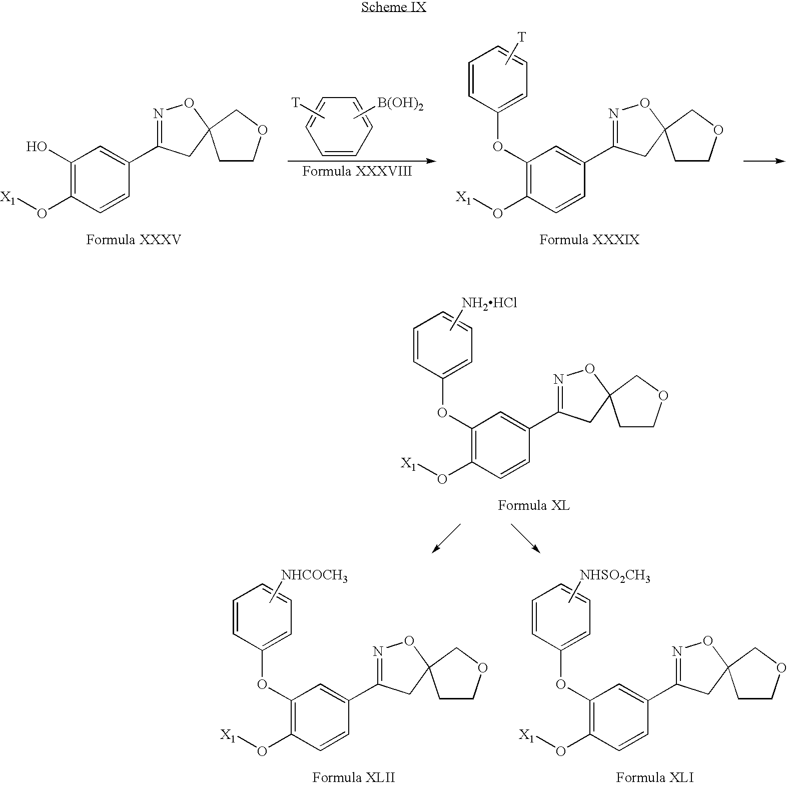

The compounds of Formulae XXXIX, XL, XLI and XLII can be prepared by following the procedure as depicted in Scheme IX. Accordingly, a compound of Formula XXXV (wherein X₁ is the same as defined earlier) can be reacted with a compound of Formula XXXVIII (wherein T can be halogen, alkoxy, alkyl or —NHCOOalkyl) to give a compound of Formula XXXIX, which (when T is —NHCOOalkyl) can be deprotected to give a compound of Formula XL, which can be

(a) mesylated to give a compound of Formula XLI.

(b) acylated to give a compound of Formula XLII.

The reaction of a compound of Formula XXXV with a compound of Formula XXXVIII to give a compound of Formula XXXIX can be carried out in the presence of a transition metal source, for example, copper acetate or elemental copper, in a solvent, for example, dichloromethane, acetonitrile or toluene.

The reaction of a compound of Formula XXXV with a compound of Formula XXXVIII to give a compound of Formula XXXIX can be carried out in the presence of a base, for example, triethyl amine, trimethyl amine, pyridine or Hunig's base.

The reaction of a compound of Formula XXXV with a compound of Formula XXXVIII to give a compound of Formula XXXIX can be carried out in the presence of for example, 4 Å molecular sieves.

The deprotection of a compound of Formula XXXIX to give a compound of Formula XL can be carried out in a solvent, for example, methanol or ethanol in the presence of a acid, for example, hydrochloric acid.

The mesylation of a compound of Formula XL to give a compound of Formula XLI can be carried out in the presence of one or more of mesylating agents, for example, methanesulfonyl chloride, methanesulfonic anhydride, trifluoromethanesulfonic anhydride, p-toluene sulphonyl chloride or mixtures thereof in the presence of a base, for example, pyridine, triethyl amine, diisopropyl ethyl amine or potassium carbonate in a solvent, for example, pyridine, dichloromethane, dichloroethane, dimethylformamide or dimethylacetamide.

The acylation of a compound of Formula XL to give a compound of Formula XLII can be carried out using acetic anhydride in a solvent, for example, pyridine, dichloromethane, dichloroethane, chloroform, dimethylformamide or dimethylacetamide.

The acylation of a compound of Formula XL to give a compound of Formula XLII can be carried out in the presence of a base, for example, pyridine, triethyl amine, diisopropyl ethyl amine or potassium carbonate.

Particular compounds prepared by this scheme are listed below:
Hydrochloride salt of 4-[2-(difluoromethoxy)-5-(1,7-dioxa-2-azaspiro[4.4]non-2-en-3-yl)phenoxy]aniline (Compound No. 124);
tert-Butyl {4-[2-(difluoromethoxy)-5-(1,7-dioxa-2-azaspiro[4.4]non-2-en-3-yl)phenoxy]phenyl}carbamate (Compound No. 125);
3-[4-(Difluoromethoxy)-3-(4-fluorophenoxy)phenyl]-1,7-dioxa-2-azaspiro[4.4]non-2-en (Compound No. 131);
3-[3-(4-Chlorophenoxy)-4-(difluoromethoxy)phenyl]-1,7-dioxa-2-azaspiro[4.4]non-2-ene (Compound No. 132);
3-{4-(Difluoromethoxy)-3-[4-(trifluoromethoxy)phenoxy]phenyl}-1,7-dioxa-2-azaspiro[4.4]non-2-ene (Compound No. 133);
3-{4-(Difluoromethoxy)-3-[4-(trifluoromethyl)phenoxy]phenyl}-1,7-dioxa-2-azaspiro[4.4]non-2-ene (Compound No. 134);
N-{4-[2-(Difluoromethoxy)-5-(1,7-dioxa-2-azaspiro[4.4]non-2-en-3-yl)phenoxy]phenyl}acetamide (Compound No. 135);
N-{4-[2-(Difluoromethoxy)-5-(1,7-dioxa-2-azaspiro[4.4]non-2-en-3-yl)phenoxy]phenyl}methane sulfonamide (Compound No. 136);
   pharmaceutically acceptable salts, pharmaceutically acceptable solvates, enantiomers, diastereomers, polymorphs or N-oxides thereof.

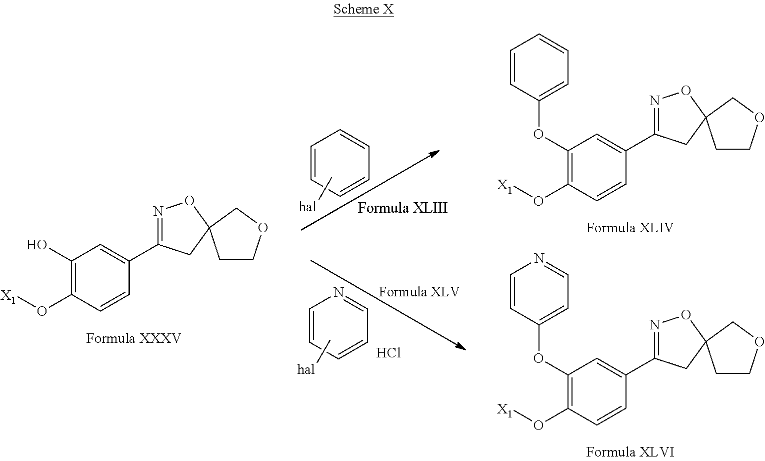

The compounds of Formulae XLIV and XLVI can be prepared by following the procedure as depicted in Scheme X. Accordingly, a compound of Formula XXVI (wherein X₁ is the same as defined earlier) can be reacted
(a) with a compound of Formula XLIII (wherein hal is the same as defined earlier) to give a compound of formula XLIV.
(b) with a compound of Formula XLV (wherein hal is the same as defined earlier) to give a compound Formula XLVI.

The reaction of a compound of Formula XXXV with a compound of Formula XLIII to give a compound of Formula XLIV can be carried out under ullmann coupling conditions, for example, in the presence of copper powder in a solvent, for example, pyridine.

The reaction of a compound of Formula XXVI with a compound of Formula XLIII to give a compound of Formula XLIV can be carried out in the presence of a base, for example, potassium carbonate or cesium carbonate.

The reaction of a compound of Formula XXXV with a compound of Formula XLV to give a compound of Formula XLVI can be carried out in the presence of a base, for example, potassium fluoride or cesium carbonate in a solvent, for example, dimethyl sulphoxide, dimethyl formamide or dimethyl acetamide.

Particular compounds prepared by this scheme are listed below:
3-[4-(Difluoromethoxy)-3-phenoxyphenyl]-1,7-dioxa-2-azaspiro[4.4]non-2-ene (Compound No. 112)
3-[4-(Difluoromethoxy)-3-(pyridin-4-yloxy)phenyl]-1,7-dioxa-2-azaspiro[4.4]non-2-ene (Compound No. 137)
   pharmaceutically acceptable salts, pharmaceutically acceptable solvates, enantiomers, diastereomers, polymorphs or N-oxides thereof.

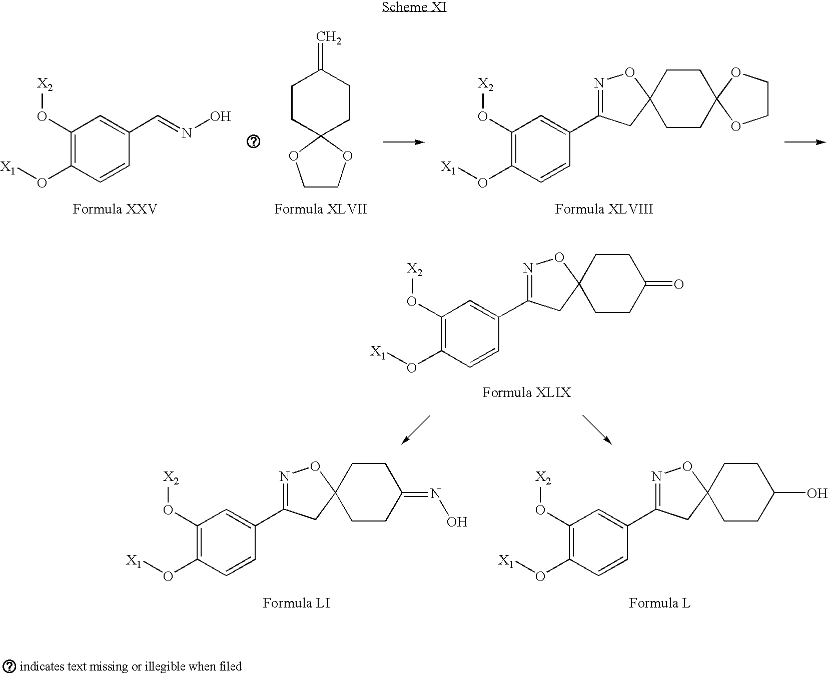

The compounds of Formulae XLVIII, XLIX, L and LI can be prepared by following the procedure as depicted in Scheme XI. Accordingly, a compound of Formula XXV (wherein X₁ and X₂ are the same as defined earlier) can be reacted with a compound of Formula XLVII to give a compound of Formula XLVIII, which can be deprotected to give a compound of Formula XLIX, which can be
(a) reduced to give a compound of Formula L.
(b) reacted with hydroxylamine hydrochloride to give a compound of Formula LI.

The reaction of a compound of Formula XXV with a compound of Formula XLVII to give a compound of Formula XLVIII can be carried out in the presence of one or more of reagents, for example, sodium hypochlorite, N-bromosuccinimide, N-chlorosuccinimide or mixtures thereof in a solvent, for example, dichloromethane, tetrahydrofuran, dimethylformamide or dimethylsulphoxide.

The deprotection of a compound of Formula XLVIII to give a compound of Formula XLIX can be carried out in the presence of one or more of acids, for example trifluoroacetic acid, p-toluene sulphonic acid or mixtures thereof in a solvent, for example, dichloromethane, water or mixtures thereof.

The reduction of a compound of Formula XLIX to give a compound of Formula L can be carried out in the presence of a reducing agent, for example, sodium borohydride, lithium aluminium hydride, sodium triacetoxy borohydride or L-selectride in a solvent, for example, tetrahydrofuran, diethyl ether, methanol, ethanol or mixtures thereof.

The reaction of a compound of Formula XLIX with hydroxylamine hydrochloride to give a compound of Formula LI can be carried out in the presence of one or more of bases, for example, alkali metal carbonates, for example, potassium carbonate or sodium carbonate, alkali metal acetates, for example, sodium acetate or mixtures thereof in a solvent, for example, dichloromethane, tetrahydrofuran, acetonitrile, dimethylformamide or mixtures thereof.

Particular compounds prepared by this scheme are listed below:
3-[4-(Difluoromethoxy)-3-methoxyphenyl]-1,9,12-trioxa-2-azadispiro[4.2.4.2]tetradec-2-ene (Compound No. 118);
3-[4-(Di fluoromethoxy)-3-methoxyphenyl]-1-oxa-2-azaspiro[4.5]dec-2-en-8-one (Compound No. 119);
3-[4-(Difluoromethoxy)-3-methoxyphenyl]-1-oxa-2-azaspiro[4.5]dec-2-en-8-one oxime (Compound No. 121);
3-[4-(Difluoromethoxy)-3-methoxyphenyl]-1-oxa-2-azaspiro[4.5]dec-2-en-8-ol (Compound No. 122);
   pharmaceutically acceptable salts, pharmaceutically acceptable solvates, enantiomers, diastereomers, polymorphs or N-oxides

Where desired, the compounds of Formula I and their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents, which may be used in combination with compounds of Formula I of this invention and their pharmaceutically acceptable salts, pharmaceutically acceptable solvates, stereoisomers, tautomers, racemates, prodrugs, metabolites, polymorphs or N-oxides include, but are not limited to, corticosteroids, β2-agonists, muscarinic receptor antagonists, anticholinergics, antiallergic agents, PAF antagonists, EGFR kinase inhibitors, p38 MAP Kinase inhibitors, additional PDE-IV inhibitors, kinase inhibitors, dopamine receptor antagonists, histamines, antitussives, leukotriene antagonists, 5-lipoxygenase inhibitors, chemokine inhibitors or combinations thereof.

The one or more β2-agonist as described herein may be chosen from those described in the art. The β2-agonists my include one or more compounds described in U.S. Pat. Nos. 3,705,233; 3,644,353; 3,642,896; 3,700,681; 4,579,985; 3,994,974; 3,937,838; 4,419,364; 5,126,375; 5,243,076; 4,992,474; and 4,011,258.

Suitable β2-agonists include, for example, one or more of albuterol, salbutamol, biltolterol, pirbuterol, levosaibutamol, tulobuterol, terbutaline, bambuterol, metaproterenol, fenoterol, salmeterol, carmoterol, arformoterol, formoterol, and their pharmaceutically acceptable salts or solvates thereof.

Corticosteroids as described herein may be chosen from those described in the art. Suitable corticosteroids may be include one or more compounds described in U.S. Pat. Nos. 3,312,590; 3,983,233; 3,929,768; 3,721,687; 3,436,389; 3,506,694; 3,639,434; 3,992,534; 3,928,326; 3,980,778; 3,780,177; 3,652,554; 3,947,478; 4,076,708; 4,124,707; 4,158,055; 4,298,604; 4,335,121; 4,081,541; 4,226,862; 4,290,962; 4,587,236; 4,472,392; 4,472,393; 4,242,334; 4,014,909; 4,098,803; 4,619,921; 5,482,934; 5,837,699; 5,889,015; 5,278,156; 5,015,746; 5,976,573; 6,337,324; 6,057,307; 6,723,713; 6,127,353; and 6,180,781. The disclosures of these patents are incorporated herein by reference in their entireties.

Suitable corticosteroids may include, for example, one or more of alclometasone, amcinonide, amelometasone, beclometasone, betamethasone, budesonide, ciciesonide, clobetasol, cloticasone, cyclomethasone, deflazacort, deprodone, dexbudesonide, diflorasone, difluprednate, fluticasone, flunisolide, halometasone, halopredone, hydrocortisone, hydrocortisone, methylprednisolone, mometasone, prednicarbate, prednisolone, rimexolone, tixocortol, triamcinolone, tolterodine, oxybutynin, ulobetasol, rofleponide, GW 215864, KSR 592, ST-126, dexamethasone and pharmaceutically acceptable salts, solvates thereof. Preferred corticosteroids include, for example, flunisolide, beclomethasone, triamcinolone, budesonide, fluticasone, mometasone, ciclesonide, and dexamethasone, while budesonide, fluticasone, mometasone, ciclesonide. Examples of possible salts or derivatives include: sodium salts, sulfobenzoates, phosphates, isonicotinates, acetates, propionates, dihydrogen phosphates, palmitates, pivalates, or furoates. In some cases, the corticosteroids may also occur in the form of their hydrates.

Suitable muscarinic receptor antagonists include substances that directly or indirectly block activation of muscarinic cholinergic receptors. Examples include, but are not limited to, the compounds disclosed in WO04/004629, WO04/005252, WO04/089900, WO04/89364, quaternary amines (e.g., methantheline, ipratropium, propantheline), tertiary amines (e.g., dicyclomine, scopolamine) and tricyclic amines (e.g., telenzepine). Other suitable muscarinic receptor antagonists include benztropine (commercially available as COGENTIN from Merck), hexahydro-sila-difenidol hydrochloride (HHSID hydrochloride disclosed in Lanibrecht et al., Trends in Pharmacol. Sci., 10(Suppl):60 (1989); (+/−)-3-quinuclidinyl xanthene-9-carboxylate hemioxalate (QNX-hemioxalate; Birdsall et al., Trends in Pharmacol, Sci., 4:459 (1983); telenzepine dihydrochloride (Coruzzi et al. Arch. Int. Pharmacodyn. Ther., 302:232 (1989); and Kawashima et al., Gen. Pharmacol., 21:17 (1990)), and atropine.

Suitable anticholinergics include, for example, tiotropium salts, ipratropium salts, oxitropitun salts, salts of the compounds known from WO 02/32899: tropenol N-methyl-2,2-diphenylpropionate, scopine N-methyl-2,2-diphenylpropionate, scopine N-methyl-2-fluoro-2,2-diphenylacetate and tropenol N-methyl-2-fluoro-2,2-diphenylacetate; as well as salts of the compounds known from WO 02/32898: tropenol N-methyl-3,3′,4,4′-tetrafluorobenzilate, scopine N-methyl-3,3′,4,4′-tetrafluorobenzilate, scopine N-methyl-4,4′-dichlorobenzilate, scopine N-methyl-4,4′-difluorobenzilate, tropenol N-methyl-3,3′-difluorobenzilate, scopine N-methyl-3,3′-difluorobenzilate, and tropenol N-ethyl-4,4′-difluorobenzilate, optionally in the form of their hydrates and solvates. By salts are meant those compounds which contain, in addition to the above mentioned cations, as counter-ion, an anion with a single negative charge selected from among the chloride, bromide, and methanesulfonate.

Preferred anticholinergics include, for example, tiotropium bromide, ipratropium bromide, oxitropium bromide, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide; scopine 4,4′-dichlorobenzilate methobromide, scopine 4,4′-difluorobenzilate methobromide, tropenol 3,3′-difluorobenzilate methobromide, scopine 3,3′-difluorobenzilate methobromide, and tropenol 4,4′-difluorobenzilate ethylbromide.

Suitable antiallergic agents include, for example, epinastine, cetirizine, azelastine, fexofenadine, levocabastine, loratadine, mizolastine, ketotifene, emedastine, dimetindene, clemastine, bamipine, hexachloropheniramine, pheniramine, doxylamine, chlorophenoxamine, dimenhydrinate, diphenhydramine, promethazine, ebastine, desloratadine, and meclizine. Preferred antiallergic agents include, for example, epinastine, cetirizine, azelastine, fexofenadine, levocabastine, loratadine, ebastine, desloratadine, and mizolastine, epinastine. Any reference to the above-mentioned antiallergic agents also includes any pharmacologically acceptable acid addition salts thereof, which may exist.

Suitable PAF antagonists include, for example, 4-(2-chlorophenyl)-9-methyl-2-[3-(4-morpholinyl)-3-propanon-1-yl]-6H-thieno[3,2-f][1,2,4]triazolo[4,3-α][1,4]diazepine and 6-(2-chlorophenyl)-8,9-dihydro-1-methyl-8-[(4-morpholinyl)carbonyl]-4H,7H-cyclopenta[4.5]thieno[3,2-f][1,2,4]triazolo[4,3-a][1,4]diazepine.

Suitable EGFR kinase inhibitors include, for example, 4-[(3-chloro-4-fluorophenyl)amino]-7-(2-{4-[(S)-(2-oxotetrahydrofuran-5-yl)carbonyl]piperazin-1-yl}-ethoxy)-6-[(vinylcarbonyl)amino]quinazoline, 4-[(3-chloro-4-fluorophenyl)amino]-7-[4-((S)-6-methyl-2-oxomorpholin-4-yl)butyloxy]-6-[(vinylcarbonyl)amino]quinazoline, 4-[(3-chloro-4-fluorophenyl)amino]-7-[4-((R)-6-methyl-2-oxomorpholin-4-yl)butyloxy]-6-[(vinylcarbonyl)amino]quinazoline, 4-[(3-chloro-4-fluorophenyl)(amino]-7-[2-((S)-6-methyl-2-oxomorpholin-4-yl)ethoxy]-6-[(vinylcarbonyl)amino]quinazoline, 4-[(3-chloro-4-fluorophenyl)amino]-6-[(4-{N-[2-(ethoxycarbonyl)ethyl]-N-[(ethoxycarbonyl)methyl]amino}-1-oxo-2-buten-1-yl)amino]-7-cyclopropylmethoxyquinazoline, 4-[(R)-(1-phenylethyl)amino]-6-{[4-(morpholin-4-yl)-1-oxo-2-buten-1-yl]amino}-7-cyclopropyl-methoxyquinazoline, and 4-[(3-chloro-4-fluorophenyl)amino]-6-[3-(morpholin-4-yl)propyloxy]-7-methoxyquinazoline. Any reference to the above-mentioned EGFR kinase inhibitors also includes any pharmacologically acceptable acid addition salts thereof which may exist. By the physiologically or pharmacologically acceptable acid addition salts thereof which may be formed by the EGFR kinase inhibitors are meant, according to the invention, pharmaceutically acceptable salts selected from among the salts of hydrochloric acid, hydrobromic acid, sulfuric acid, phosphoric acid, methanesulfonic acid, acetic acid, fumaric acid, succinic acid, lactic acid, citric acid, tartaric acid, or maleic acid. The salts of the EGFR kinase inhibitors selected from among the salts of acetic acid, hydrochloric acid, hydrobromic acid, sulfuric acid, phosphoric acid, and methanesulfonic acid are preferred according to the invention.

Suitable p38 kinase inhibitors include, for example, 1-[5-tert-butyl-2-p-tolyl-2H-pyrazol-3-yl]-3-[4-(2-morpholin-4-ylethoxy)naphthalen-1-yl]urea; 1-[5-tert-butyl-2-p-tolyl-2H-pyrazol-3-yl]-3-[4-(2-(1-oxothiomorpholin-4-yl)ethoxy)naphthalen-1-yl]urea; 1-[5-tert-butyl-2-(2-methylpyridin-5-yl)-2H-pyrazol-3-yl]-3-[4-(2-pyridin-4-ylethoxy)naphthalen-1-yl]urea; 1-[5-tert-butyl-2-(2-methoxypyridin-5-yl)-2H-pyrazol-3-yl]-3-[4-(2-morpholin-4-ylethoxy)naphthalen-1-yl]urea; and 1-[5-tert-butyl-2-methyl-2H-pyrazol-3-yl]-3-[4-(2-morpholin-4-ylethoxy)naphthalen-1-yl]urea disclosed in our co-pending U.S. patent application No. 60/605,344; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid tert-butyl ester; Hydrochloride salt of 2-(Piperidin-4-ylamino)-8-(tetrahydro-pyran-4-yl)-6-o-tolyl-8H-pyrido[2,3-d]pyrimidin-7-one; 2-(1-Methanesulfonyl-piperidin-4-ylamino)-8-(tetrahydro-pyran-4-yl)-6-o-tolyl-8H-pyrido[2,3-d]pyrimidin-7-one; 1-Benzyl-piperidin-4-ylamino)-8-(tetrahydro-pyran-4-yl)-6-o-tolyl-8H-pyrido[2,3-d]pyrimidin-7-one; 2-(1-Methyl-piperidin-4-ylamino)-8-(tetrahydro-pyran-4-yl)-6-o-tolyl-8H-pyrido[2,3-d]pyrimidin-7-one; 2-(4-Methyl-piperazin-1-ylamino)-8-(tetrahydro-pyran-4-yl)-6-o-tolyl-8H-pyrido[2,3-d]pyrimidin-7-one; 4-[6-(2-Chloro-phenyl)-7-oxo-8-(tetrahydro-pyran-4-yl)-7,8-dihydro-pyrido[2,3-d]pyrimidin-2-ylamino]-piperidine-1-carboxylic acid tert-butyl ester; 2-(Piperidin-1-ylamino)-8-(tetrahydro-pyran-4-yl)-6-o-tolyl-8H-pyrido[2,3-d]pyrimidin-7-one; 2-Cyclobutylamino-8-(tetrahydro-pyran-4-yl)-6-o-tolyl-8H-pyrido[2,3-d]pyrimidin-7-one; 2-(1-Acetyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 2-(1-Benzoyl-piperidin-4-ylamino)-8-(tetrahydro-pyran-4-yl)-6-o-tolyl-8H-pyrido[2,3-d]pyrimidin-7-one; 4-[7-oxo-8-(tetrahydro-pyran-4-yl)-6-o-tolyl-7,8-dihydro-pyrido[2,3-d]pyrimidin-2-ylamino]-piperidine-1-carboxylic acid (4-fluoro-phenyl)-amide; 2-(1-Ethanesulfonyl-piperidin-4-ylamino)-8-(tetrahydro-pyran-4-yl)-6-o-tolyl-8H-pyrido[2,3-d]pyrimidin-7-one; 4-[7-oxo-8-(tetrahydro-pyran-4-yl)-6-o-tolyl-7,8-dihydro-pyrido[2,3-d]pyrimidin-2-ylamino]-piperidine-1-carbothioic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino]-piperidine-1-carboxylic acid (4-trifluoromethyl-phenyl)-amide; 2-[4-(Propane-2-sulfonyl)-piperazin-1-ylamino]-8-(tetrahydro-pyran-4-yl)-6-o-tolyl-8H-pyrido[2,3-d]pyrimidin-7-one; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid propylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid ((R)-1,2-dimethyl-propyl)-amide; 4-[7-Oxo-8-(tetrahydro-pyran-4-yl-6-o-tolyl-7,8-dihydro-pyrido[2,3-d]pyrimidin-2-ylamino]-piperazine-1-carboxylic acid cyclohexylamide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid (4-fluoro-phenyl)-amide; 4-[7-Oxo-8-(tetrahydro-pyran-4-yl)-6-o-tolyl-7,8-dihydro-pyrido[2,3-d]pyrimidin-2-ylamino]-piperazine-1-carboxylic acid cyclopentyl methyl-amide; and the compounds which are disclosed in our co-pending U.S. patent application No. 60/598,621, 60/630,517, and Indian patent application no, 1098/DEL/2005 and 211/DEL/2005. Any reference to the above mentioned p38 kinase inhibitors also includes any pharmacologically acceptable acid addition salts thereof. According to the invention, physiologically or pharmacologically acceptable acid addition salts thereof of the p38 kinase inhibitors are include salts with hydrochloric acid, hydrobromic acid, sulfuric acid, phosphoric acid, methanesulfonic acid, acetic acid, fumaric acid, succinic acid, lactic acid, citric acid, tartaric acid, and maleic acid.

The leukotriene antagonist can be selected from compounds not limited to those described in U.S. Pat. No. 5,565,473, U.S. Pat. No. 5,583,152, U.S. Pat. No. 4,859,692 or U.S. Pat. No. 4,780,469.

Examples of leukotriene antagonist include, but are not limited to, montelukast, zafirlukast, pranlukast and pharmaceutically acceptable salts thereof.

5-Lipoxygenase inhibitors can be selected from the compounds disclosed in U.S. Pat. Nos. 4,826,868, 4,873,259, EP 419049, EP 542356 or EP 542355. Examples may include but are not limited to atreleuton, zyflo (zileuton), ABT-761, fenleuton or tepoxalin.

Chemokine inhibitors can be selected from the compounds disclosed in EP 287436, EP 389359, EP 988292, WO 02/26723 or WO 01/90106.

Examples of chemokine inhibitors include, but are not limited to AMD3100, AZD 8309, BX-471, GW-766994, UK-427857, CP-481715, UK-107543, UK-382055 or UK-395859.

Because of their valuable pharmacological properties, the compounds described herein may be administered to an animal for treatment orally, by inhalation, by intranasal route, rectally, parenterally (intravenously, intramuscularly or subcutaneously), intracistemally, intratracheally, intravaginally, intraperitoneally or topically.

The pharmaceutical compositions described herein can be produced and administered in dosage units, each unit containing a certain amount of at least one compound described herein and at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their racemic mixtures, enantiomers, diastereomers, rotamers, N-oxides, polymorphs, solvates and pharmaceutically acceptable salts, as well as the active metabolites. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

The examples mentioned below demonstrate general synthetic procedures, as well as specific preparations of particular compounds. The examples are provided to illustrate the details of the invention and should not be constrained to limit the scope of the present invention.

### EXPERIMENTAL

### General Procedure

### Synthesis of a Compound of Formula VI

### Step a: Formula II

To a solution of the compound (S or R)-3-[3-(cycloalkyloxy)-4-alkoxyphenyl]-1,7-dioxa-2-azaspiro[4.4]non-2-ene (2 g) (prepared by following the procedure described in WO 2006/085212) in dichloromethane was added aluminium trichloride (1.68 g) at 0° C. and stirred the reaction mixture at room temperature for 2 hours. The reaction mixture was subsequently poured into ice-cold water and extracted with dichloromethane, washed with water and brine, dried over anhydrous sodium sulphate and concentrated under reduced pressure to furnish the title compound.

The following compounds were prepared by following the above procedure
5-[(5S or 5R)-1,7-Dioxa-2-azaspiro[4.4]non-2-en-3-yl]-2-methoxyphenol (Compound No. 6)

Mass (m/z): 250.03 (M⁺+1).
5-[(5R or 5S)-1,7-Dioxa-2-azaspiro[4.4]non-2-en-3-yl]-2-methoxyphenol (Compound No. 92)

Mass: 250.27 (M⁺+1).

### Step b: Formula IV

To a solution of the compound obtained from step a above (2.5 g) in dimethyl formamide was added the compound of Formula III (1.8 mL) and potassium carbonate (2.77 g). The reaction mixture was stirred at 50-60° C. overnight. To the resulting reaction mixture was added water and extracted with ethyl acetate. The organic layer was separated, washed with water and brine, dried over anhydrous sodium sulphate and concentrated under reduced pressure to furnish the title compound.

The following compounds were prepared by following the above procedure
(5S or 5R)-3-(3,4-Dimethoxyphenyl)-1,7-dioxa-2-azaspiro[4.4]non-2-ene (Compound No. 7);

Mass (m/z): 263.97 (M⁺+1).
(5R or 5S)-3-(3,4-Dimethoxyphenyl)-1,7-dioxa-2-azaspiro[4.4]non-2-ene (Compound No. 8);
(5S or 5R)-3-(3-isopropoxy-4-methoxyphenyl)-1,7-dioxa-2-azaspiro[4.4]non-2-ene (Compound No. 57);

Mass (m/z); 292 (M⁺+1).
(5S or 5R)-3-[3-(Cyclopropylmethoxy)-4-methoxyphenyl]-1,7-dioxa-2-azaspiro[4.4]non-2-ene (Compound No. 58);

Mass (m/z): 304 (M⁺+1).
(5S or 5R)-3-[3-(Benzyloxy)-4-methoxyphenyl]-1,7-dioxa-2-azaspiro[4.4]non-2-ene (Compound No. 78);

Mass (m/z): 340.30 (M⁺+1).
(5R or 5S)-3-[3-(benzyloxy)-4-methoxyphenyl]-1,7-dioxa-2-azaspiro[4.4]non-2-ene (Compound No. 126);

Mass (m/z: 340.05 (M⁺+1).

### Step c: Formula V

To a solution of the compound obtained from step b above (500 mg) in dimethyl acetamide under nitrogen atmosphere was added sodium ethane thiolate (505 mg) and stirred the reaction mixture at 110-120′C for about 3 hours. Excess of dimethyl acetamide was evaporated under reduced pressure and the reaction mixture was acidified with ammonium chloride solution. The mixture was extracted with ethyl acetate, washed with water and brine, dried over anhydrous sodium sulphate and concentrated under reduced pressure. The residue thus obtained was purified by column chromatography to furnish the title compound.

The following compounds were prepared by following the above procedure
2-(Benzyloxy)-4-(1,7-dioxa-2-azaspiro[4.4]non-2-en-3-yl)phenol (Compound No. 9

Mass (m/z): 325.94 (M⁺+1).
4-[(5S or 5R)-1,7-dioxa-2-azaspiro[4.4]non-2-en-3-yl]-2-methoxyphenol (Compound No. 4);

Mass (m/z): 250.02 (M⁺+1).
4-[(5R or 5S)-1,7-dioxa-2-azaspiro[4.4]non-2-en-3-yl]-2-methoxyphenol (Compound No. 5);

Mass (m/z): 250.02 (M⁺+1).
2-(Cyclopropylmethoxy)-4-[(5S or 5R)-1,7-dioxa-2-azaspiro[4.4]non-2-en-3-yl]phenol (Compound No. 59);

Mass (m/z): 290 (M⁺+1).
4-[(5S or 5R)-1,7-Dioxa-2-azaspiro[4.4]non-2-en-3-yl]-2-isopropoxyphenol (Compound No. 60);

Mass (m/z): 278 (M⁺+1).
2-(Cyclopropylmethoxy)-4-[(5R or 5S)-1,7-dioxa-2-azaspiro[4.4]non-2-en-3-yl]phenol (Compound No. 65);
4-[(5R or 5S)-1,7-Dioxa-2-azaspiro[4.4]non-2-en-3-yl]-2-isopropoxyphenol (Compound No. 66);
2-(Benzyloxy)-4-[(5S or 5R)-1,7-dioxa-2-azaspiro[4.4]non-2-en-3-yl]phenol (Compound No. 77);
2-(Benzyloxy)-4-[(5R or 5S)-1,7-dioxa-2-azaspiro[4.4]non-2-en-3-yl]phenol (Compound No. 127);

Mass (m/z): 325.99 (M⁺+1).

### Step d: Formula VI

To a mixture of the compound obtained from step c above (370 mg) and potassium carbonate (369 mg) in dimethyl formamide was added the compound of Formula IIIa and stirred the reaction mixture overnight. To the resulting mixture water was added, extracted with ethyl acetate, washed with water and brine and dried over anhydrous sodium sulphate and concentrated under reduced pressure. The residue thus obtained was purified by column chromatography to furnish the title compound.

The following compounds were prepared by following the above procedure
(5R or 5S)-3-[4-(Difluoromethoxy)-3-methoxyphenyl]-1,7-dioxa-2-azaspiro[4.4]non-2-ene (Compound No. 13);

Mass (m/z): 300 (M⁺+1),
(5S or 5R)-3-[4-(Difluoromethoxy)-3-methoxyphenyl]-1,7-dioxa-2-azaspiro[4.4]non-2-ene (Compound No. 14);

Mass (m/z): 299.98 (M⁺+1),
(5S or 5R)-3-[3-(Cyclopropylmethoxy)-4-(difluoromethoxy)phenyl]-1,7-dioxa-2-azaspiro[4.4]non-2-ene (Compound No. 62);

Mass m/z): 340 (M⁺+1).
(5S or 5R)-3-[4-(difluoromethoxy)-3-isopropoxyphenyl]-1,7-dioxa-2-azaspiro[4.4]non-2-ene (Compound No. 63);

Mass (m/z): 328 (M⁺+1).
(5R or 5S)-3-[4-(difluoromethoxy)-3-isopropoxyphenyl]-1,7-dioxa-2-azaspiro[4.4]non-2-ene (Compound No. 64)

Mass (m/z): 292 (M⁺+1).
(5R or 5S)-3-[3-(Cyclopropylmethoxy)-4-(difluoromethoxy)phenyl]-1,7-dioxa-2-azaspiro[4.4]non-2-ene (Compound No. 67);

Mass (m/z): 340.24 (M⁺+1).
(5R or 5S)-3-[4-(difluoromethoxy)-3-isopropoxyphenyl]-1,7-dioxa-2-azaspiro[4.4]non-2-ene (Compound No. 68);

Mass (m/z): 328.26 (M⁺+1),
(5S or 5R)-3-[3-(Benzyloxy)-4-(difluoromethoxy)phenyl]-1,7-dioxa-2-azaspiro[4.4]non-2-ene (Compound No. 76);

Mass (m/z): 376 (M⁺+1).
3-[3-(Benzyloxy)-4-(2,2,2-trifluoroethoxy)phenyl]-1,7-dioxa-2-azaspiro[4.4]non-2-ene (Compound No. 90);

Mass (m/z): 408.8 (M⁺+1).
(5R or 5S)-3-[3-(Benzyloxy)-4-(difluoromethoxy)phenyl]-1,7-dioxa-2-azaspiro[4.4]non-2-ene (Compound No. 128);

Mass (m/z): 376.02 (M⁺+1).

### Synthesis of a Compound of Formula XI

### Step a: Formula VII

To a solution of the compound of Formula VI (1.0 eq.) in methanol was added palladium on carbon (10% by weight) and hydrogen gas was purged through baloon in the reaction mixture. The reaction mixture was stirred overnight. The mixture was filtered through celite pad. The filtrate was concentrated under reduced pressure. The residue thus obtained was purified by column chromatography to furnish the title compound.

The following compounds were prepared by following the above procedure
5-(1,7-Dioxa-2-azaspiro[4.4]non-2-en-3-yl)-2-(2,2,2-trifluoroethoxy)phenol (Compound No. 33);

Mass (m/z): 318.11 (M⁺+1).
2-(Difluoromethoxy)-5-[(5S or 5R)-1,7-dioxa-2-azaspiro[4.4]non-2-en-3-yl]phenol (Compound No. 91);

Mass (m/z): 286.1 (M⁺+1).
2-(Difluoromethoxy)-5-[(5R or 5S)-1,7-dioxa-2-azaspiro[4.4]non-2-en-3-yl]phenol (Compound No. 130);

Mass (m/z): 286.09 (M⁺+1).

### Step b: Formula XI

The title compound was prepared following the procedure as described for the preparation of Formula IV by reacting the compound of Formula. VIE with a compound of Formula. X

The following compounds were prepared by following the above procedure.
3-[2-(Difluoromethoxy)-5-(1,7-dioxa-2-azaspiro[4.4]non-2-en-3-yl)phenoxy]propan-1-ol (Compound No. 2);

Mass (m/z); 344.10 (M⁺+1).
[2-(Difluoromethoxy)-5-(1,7-dioxa-2-azaspiro[4.4]non-2-en-3-yl)phenoxy]acetonitrile (Compound No. 3);

Mass (m/z): 347.01 (M⁺+23).
2-[2-(Difluoromethoxy)-5-(1,7-dioxa-2-azaspiro[4.4]non-2-en-3-yl)phenoxy]ethanol (Compound No. 10);

Mass (m/z): 330.11 (M⁺+1),
3-[4-(Difluoromethoxy)-3-ethoxyphenyl]-1,7-dioxa-2-azaspiro[4.4]non-2-ene (Compound No. 11);

Mass (m/z): 314.06 (M⁺+1).
3-[3-(Cyclohexyloxy)-4-(difluoromethoxy)phenyl]-1,7-dioxa-2-azaspiro[4.4]non-2-ene (Compound No. 12);
Ethyl [2-(difluoromethoxy)-5-(1,7-dioxa-2-azaspiro[4.4]non-2-en-3-yl)phenoxy]acetate (Compound No. 15);

Mass (m/z): 372.04 (M⁺+1).
3-[4-(Difluoromethoxy)-3-(2-morpholin-4-ylethoxy)phenyl]-1,7-dioxa-2-azaspiro[4.4]non-2-ene (Compound No. 16);

Mass (m/z): 399.06 (M⁺+1),
5-[2-(Difluoromethoxy)-5-(1,7-dioxa-2-azaspiro[4.4]non-2-en-3-yl)phenoxy]pentanoic acid (Compound No. 18);

Mass (m/z): 386.09 (M⁺+1).
3-[3-(2,2,2-Trifluoroethoxy)-4-(difluoromethoxy)phenyl]-1,7-dioxa-2-azaspiro[4.4]non-2-one (Compound No. 19);

Mass (m/z): 368.04 (M⁺+1).
3-[3-(Cyclopentylmethoxy)-4-(difluoromethoxy)phenyl]-1,7-dioxa-2-azaspiro[4.4]non-2-ene (Compound No. 20);

Mass (m/z): (M⁺+1).
3-[3-(cyclopentyloxy)-4-(2,2,2-trifluoromethoxy)phenyl]-1,7-dioxa-2-azaspiro[4.4]non-2-ene (Compound No. 24);

Mass (m/z): 386 (M⁺+1).
3-[3-Propoxy-4-(2,2,2-trifluoroethoxy)phenyl]-1,7-dioxa-2-azaspiro[4.4]non-2-ene (Compound No. 29);

Mass (m/z): 360 (M⁺+1).
3-[3-Isopropoxy-4-(2,2,2-trifluoroethoxy)phenyl]-1,7-dioxa-2-azaspiro[4.4]non-2-ene (Compound No. 30);

Mass (m/z): 360 (M⁺+1).
3-[3-(Cyclopropylmethoxy)-4-(2,2,2-trifluoroethoxy)phenyl]-1,7-dioxa-2-azaspiro[4.4]non-2-ene (Compound No. 31);

Mass (m/z): 372 (M⁺+1).
3-[3-(2,3-Dihydro-1H-inden-2-yloxy)-4-(2,2,2-trifluoroethoxy)phenyl]-1,7-dioxa-2-azaspiro[4.4]non-2-ene (Compound No. 32);

Mass (m/z): 434 (M⁺+1).
3-[3-Methoxy-4-(2,2,2-trifluoroethoxy)phenyl]-1,7-dioxa-2-azaspiro[4.4]non-2-ene (Compound No. 34);

Mass (m/z): 332 (M⁺+1).
3-[3-Ethoxy-4-(2,2,2-trifluoroethoxy)phenyl]-1,7-dioxa-2-azaspiro[4.4]non-2-ene (Compound No. 35);

Mass (m/z): 346 (M⁺+1).
3-[3-Butoxy-4-(2,2,2-trifluoroethoxy)phenyl]-1,7-dioxa-2-azaspiro[4.4]non-2-ene (Compound No. 36);
3-[3-(Cyclohexylmethoxy)-4-(2,2,2-trifluoroethoxy)phenyl]-1,7-dioxa-2-azaspiro[4.4]non-2-ene (Compound No. 37);

Mass (m/z): 414 (M⁺+1).
3-{[2-(Difluoromethoxy)-5-(1,7-dioxa-2-azaspiro[4.4]non-2-en-3-yl)phenoxy]methyl}benzonitrile (Compound No. 38);

Mass (m/z): 401 (M⁺+1).

2-{2-[2-(Difluoromethoxy)-5-(1,7-dioxa-2-azaspiro[4.4]non-2-en-3-yl)phenoxy]ethyl}-1H-isoindole-1,3(2H)-dione (Compound No. 39);

Mass (m/z): 459 (M⁺+1).
3-[3-(Cyclohexyloxy)-4-(2,2,2-trifluoroethoxy)phenyl]-1,7-dioxa-2-azaspiro[4.4]non-2-ene (Compound No. 40);

Mass (m/z): 400 (M⁺+1).
Ethyl [5-(1,7-dioxa-2-azaspiro[4.4]non-2-en-3-yl)-2-(2,2,2-trifluoroethoxy)phenoxy]acetate (Compound No. 41);

Mass (m/z): 404 (M⁺+1).
3-[3-(Cyclohexylmethoxy)-4-(difluoromethoxy)phenyl]-1,7-dioxa-2-azaspiro[4.4]non-2-ene (Compound No. 42);

Mass (m/z): 382 (M⁺+1).
Tert-butyl [2-(difluoromethoxy)-5-(1,7-dioxa-2-azaspiro[4.4]non-2-en-3-yl)phenoxy]acetate (Compound No. 43);

Mass (m/z): 400 (M⁺+1).

### Synthesis of a Compound of Formula IX

To a solution of a compound of Formula VII (50 mg) in dimethylformamide (5 mL) was added sodium hydride (16 mg) at 0° C. and stirred the reaction mixture for 1 hour followed by the addition of the compound of Formula VIII (0.02 mL). The reaction mixture was stirred at room temperature for 4 hours followed by the addition of water. The compound was extracted with ethyl acetate, washed with brine solution, dried over anhydrous sodium sulphate and concentrated under reduced pressure to furnish the title compound.

The following compounds were prepared by following the above procedure
2-(Difluoromethoxy)-5-(1,7-dioxa-2-azaspiro[4.4]non-2-en-3-yl)phenyl cyclohexanecarboxylate (Compound No. 17)

Mass (m/z): 396.08 (M⁺+1),
2-(Difluoromethoxy)-5-(1,7-dioxa-2-azaspiro[4.4]non-2-en-3-yl)phenyl cyclopropanecarboxylate (Compound No. 25);

Mass (m/z): 354.09 (M⁺+1).
2-(Difluoromethoxy)-5-(1,7-dioxa-2-azaspiro[4.4]non-2-en-3-yl)phenyl morpholine-4-carboxylate (Compound No. 26);

Mass (m/z): 399.1.5 (M⁺+1).
2-(Difluoromethoxy)-5-(1,7-dioxa-2-azaspiro[4.4]non-2-en-3-yl)phen yl benzoate (Compound No. 27);

Mass (m/z); 390.13 (M⁺+1).

### Synthesis of a Compound of Formula XIII

### Step a: Formula XI

The title compound was prepared following the procedure as described for the preparation compound of Formula. IV, by reacting the compound of Formula VII with a compound of Formula X.

### Step b: Formula XIII

To a solution of the compound of Formula XI (wherein R_{3y} is —(CH₂)_{g1}C(═O)OR₃) (0.060 g) in dichloromethane (4 mL) was added the compound of Formula XII (2 mL) and stirred the reaction mixture for 3 to 4 hours at 50-60° C. The reaction mixture was washed with dilute hydrochloric acid and water. The organic layer was dried over anhydrous sodium sulphate and concentrated under reduced pressure to furnish the title compound. The following compounds were prepared by following the above procedure
N-cyclopropyl-2-[2-(difluoromethoxy)-5-(1,7-dioxa-2-azaspiro[4.4]non-2-en-3-yl)phenoxy]acetamide (Compound No. 21);

Mass (m/z): 383.08 (M⁺+1).
2-[2-(Difluoromethoxy)-5-(1,7-dioxa-2-azaspiro[4.4]non-2-en-3-yl)phenoxy]acetamide (Compound No. 22);

Mass (m/z): 343.09 (M⁺+1),
2-[2-(Difluoromethoxy)-5-(1,7-dioxa-2-azaspiro[4.4]non-2-en-3-yl)phenoxy]-N-methylacetamide (Compound No. 23);

Mass (m/z): 357.06 (M⁺+1).
5-[2-(Difluoromethoxy)-5-(1,7-dioxa-2-azaspiro[4.4]non-2-en-3-yl)phenoxy]pentanamide (Compound No. 28);

Mass (m/z): 385.20 (M⁺+1).
N-cyclopropyl-2-[5-(1,7-dioxa-2-azaspiro[4.4]non-2-en-3-yl)-2-(2, trifluoroethoxy)phenoxy]acetamide (Compound No. 44);

Mass (m/z): 415 (M⁺+1).
N-benzyl-2-[5-(1,7-dioxa-2-azaspiro[4.4]non-2-en-3-yl)-2-(2,2,2-trifluoroethoxy)phenoxy]acetamide (Compound No. 47);

Mass (m/z): 465 (M⁺+1).
N-cyclopentyl-2-[5-(1,7-dioxa-2-azaspiro[4.4]non-2-en-3-yl)-2-(2,2,2-trifluoroethoxy)phenoxy]acetamide (Compound No. 48);

Mass (m/z): 443 (M⁺+1).

### Scheme II:

### General Procedure:

### Synthesis of a Compound of Formula IVa

### Step a: Formula IIa

To a solution of the compound (S or R)-3-[3-(cycloalkyloxy)-4-alkoxyphenyl]-1,7-dioxa-2-azaspiro[4.4]non-2-ene (510 mg) (prepared by following the procedure described in WO 2006/085212) under nitrogen atmosphere in dimethylacetamide was added sodium ethane thiolate (473 mg) and stirred the reaction mixture at 110-120° C. for 5-6 hours. Excess of dimethyl acetamide was evaporated under reduced pressure and the residue thus Obtained was acidified by ammonium chloride solution. The mixture was extracted with ethyl acetate, washed with water and brine, dried over anhydrous sodium sulphate and concentrated under reduced pressure. The residue thus obtained was purified by column chromatography to furnish the title compound.

### Step b: Formula IVa

To a solution of the compound obtained from step a above (400 mg) in dimethyl formamide was added potassium carbonate (364 mg) and a compound of Formula over a period of 10 minutes. The reaction mixture was stirred overnight which was thereafter diluted with water and extracted with ethyl acetate. The organic layer was washed with water and brine, dried over anhydrous sodium sulphate and concentrated under reduced pressure. The residue thus obtained was purified by column chromatography to furnish the title compound.

The following compounds were prepared by following the above procedure:
2-(Cyclopentyloxy)-4-[(5R or 5S)-1,7-dioxa-2-azaspiro[4.4]non-2-en-3-yl]phenol (Compound No. 45);

Mass (m/z): 304 (M⁺+1).
2-(Cyclopentyloxy)-4-[(5S or 5R)-1,7-dioxa-2-azaspiro[4.4]non-2-en-3-yl]phenol (Compound No. 46);

Mass (m/z): 304 (M⁺+1).
(5R or 5S)-3-[3-(cyclopentyloxy)-4-(difluoromethoxy)phenyl]-1,7-dioxa-2-azaspiro[4.4]non-2-ene (Compound No. 56);

Mass (m/z): 354 (M⁺+1).
(5S or 5R)-3-[3-(cyclopentyloxy)-4-(difluoromethoxy)phenyl]-1,7-dioxa-2-azaspiro[4.4]non-2-ene (Compound No. 61);

Mass (m/z): 354 (M⁺+1).

### Scheme III:

### General Procedure:

### Synthesis of a Compound of Formula XVI

To a solution of the compound of Formula XIV (450 mg) in dimethylformamide was added a compound of Formula XV (728 mg) and potassium carbonate (653 mg). The reaction mixture was stirred for 4 hours at 60-70° C. The reaction mixture was diluted with water and was extracted with ethyl acetate. The organic layer was separated, dried over anhydrous sodium sulphate, filtered and concentrated under reduced pressure. The residue thus obtained was purified by preparative thin layer chromatography to furnish the title compound.

The following compounds were prepared by following the above procedure
3-[3-{[(3S)-1-Benzylpyrrolidin-3-yl]oxy}-4-(difluoromethoxy)phenyl]-1,7-dioxa-2-azaspiro[4.4]non-2-ene (Compound No. 1);

Mass (m/z): 445 (M⁺+1).
Tert-butyl (3S)-3-[2-(difluoromethoxy)-5-(1,7-dioxa-2-azaspiro[4.4]non-2-en-3-yl)phenoxy]pyrrolidine-1-carboxylate (Compound No. 52);

Mass (m/z): 455 (M⁺+1).
Tert-butyl 4-[2-(difluoromethoxy)-5-(1,7-dioxa-2-azaspiro[4.4]non-2-en-3-yl)phenoxy]piperidine-1-carboxylate (Compound No. 49);

Mass (m/z): 369 (M⁺+1-Boc).
Tert-butyl (3R)-3-[2-(difluoromethoxy)-5-(1,7-dioxa-2-azaspiro[4.4]non-2-en-3-yl)phenoxy]pyrrolidine-1-carboxylate (Compound No. 53);

Mass (m/z): 455 (M⁺+1).
Tert-butyl 3-[2-(difluoromethoxy)-5-(1,7-dioxa-2-azaspiro[4.4]non-2-en-3-yl)phenoxy]piperidine-1-carboxylate (Compound No. 54);

Mass (m/z): 469 (M⁺+1).
Tert-butyl (2S)-2-{[2-(difluoromethoxy)-5-(1,7-dioxa-2-azaspiro[4.4]non-2-en-3-yl)phenoxy]methyl}pyrrolidine-1-carboxylate (Compound No. 55);

Mass (m/z): 469.34 (M⁺+1).

### Synthesis of a Compound of Formula XVII

A solution of the compound of Formula XVI (150 mg) in methanolic HCl (3.0 mL) was stirred at room temperature for 3 hours. The solvent was evaporated under reduced pressure and dried under high vacuum to furnish the title compound.

The following compounds were prepared by following the above procedure
Hydrochloride salt of 3-[4-(difluoromethoxy)-3-(piperidin-4-yloxy)phenyl]-1,7-dioxa-2-azaspiro[4.4]non-2-ene (Compound No. 50);

Mass (m/z): 369 (M⁺+1).
Hydrochloride salt of 3-{4-(Difluoromethoxy)-3-[(3S)-pyrrolidin-3-yloxy]phenyl}-1,7-dioxa-2-azaspiro[4.4]non-2-ene (Compound No. 69);

Mass (m/z): 355 (M⁺+1).
Hydrochloride salt of 3-{4-(difluoromethoxy)-3-[(2S)-pyrrolidin-2-ylmethoxy]phenyl}-1,7-dioxa-2-azaspiro[4.4]non-2-ene (Compound No. 70);

Mass (m/z): 369 (M⁺+1).
Hydrochloride salt of 3-{4-(difluoromethoxy)-3-[(2R)-pyrrolidin-2-ylmethoxy]phenyl}-1,7-dioxa-2-azaspiro[4.4]non-2-ene (Compound No. 71);

Mass (m/z): 369 (M⁺+1).
Hydrochloride salt of 3-[4-(difluoromethoxy)-3-(piperidin-3-yloxy)phenyl]-1,7-dioxa-2-azaspiro[4.4]non-2-ene (Compound No. 94);

Mass (m/z): 369.34 (M⁺+1).

### Synthesis of a Compound of Formula XIX

To a solution of the compound of Formula XVII (50 mg) in dimethylformamide was added potassium carbonate (75 mg) and a compound of Formula XVIII (21 mg). The reaction mixture was stifled overnight. The mixture was diluted with water and extracted with ethyl acetate. The organic layer was separated, dried over anhydrous sodium sulphate, filtered and concentrated under reduced pressure. The residue thus obtained was purified by preparative thin layer chromatography to furnish the title compound.

The following compounds were prepared by following the above procedure
3-{3-[(1-Acetylpiperidin-4-yl)oxy]-4-(difluoromethoxy)phenyl}-1,7-dioxa-2-azaspiro[4.4]non-2-ene (Compound No. 51);

Mass (m/z): 411 (M⁺+1).
3-[4-(Difluoromethoxy)-3-{[(2R)-1-propionylpyrrolidin-2-yl]methoxy}phenyl]-1,7-dioxa-2-azaspiro[4.4]non-2-ene (Compound No. 72);

Mass (m/z): 425 (M⁺+1).
3-[3-{[(2S)-1-Acetylpyrrolidin-2-yl]methoxy}-4-(difluoromethoxy)phenyl]-1,7-dioxa-2-azaspiro[4.4]non-2-ene (Compound No. 73);

Mass (m/z): 411 (M⁺+1).
3-[3-{[(3S)-1-Benzoylpyrrolidin-3-yl]oxy}-4-(difluoromethoxy)phenyl]-1,7-dioxa-2-azaspiro[4.4]non-2-ene (Compound No. 74);

Mass (m/z): 459 (M⁺+1).
3-[4-(Difluoromethoxy)-3-{[(3S)-1-propionylpyrrolidin-3-yl]oxy}phenyl]-1,7-dioxa-2-azaspiro[4.4]non-2-ene (Compound No. 75);

Mass (m/z): 410 (M⁺+1),
3-{4-(Difluoromethoxy)-3-[(1-propionylpiperidin-4-yl)oxy]phenyl}-1,7-dioxa-2-azaspiro[4.4]non-2-ene (Compound No. 79);

Mass (m/z): 425 (M⁺+1).
3-[4-(Difluoromethoxy)-3-{[1-(4-fluorobenzoyl)piperidin-4-yl]oxy}phenyl]-1,7-dioxa-2-azaspiro[4.4]non-2-ene (Compound No. 80);

Mass (m/z): 491 (M⁺+1),
3-[3-{[1-(Cyclopropylcarbonyl)piperidin-4-yl]oxy}-4-(difluoromethoxy)phenyl]-1,7-dioxa-2-azaspiro[4.4]non-2-ene (Compound No. 81);

Mass (m/z): 437 (M⁺+1).
3-[3-{[1-(Cyclopentylcarbonyl)piperidin-4-yl]oxy}-4-(difluoromethoxy)phenyl]-1,7-dioxa-2-azaspiro[4.4]non-2-ene (Compound No. 82);

Mass (m/z): 465 (M⁺+1).
3-[4-(Difluoromethoxy)-3-({1-[(trifluoromethyl)sulfonyl]piperidin-4-yl}oxy)phenyl]-1,7-dioxa-2-azaspiro[4.4]non-2-ene (Compound No. 83);

Mass (m/z): 501 (M⁺+1).
3-{3-[(1-Acetylpiperidin-3-yl)oxy]-4-(difluoromethoxy)phenyl}-1,7-dioxa-2-azaspiro[4.4]non-2-ene (Compound No. 84);

Mass (m/z): 411 (M⁺+1).
3-{4-(Difluoromethoxy)-3-[(1-propionylpiperidin-3-yl)oxy]phenyl}-1,7-dioxa-2-azaspiro[4.4]non-2-ene (Compound No. 85);

Mass (m/z): 425 (M⁺+1).
3-[4-(Difluoromethoxy)-3-{[1-(4-fluorobenzoyl)piperidin-3-yl]oxy}phenyl]-1,7-dioxa-2-azaspiro[4.4]non-2-ene (Compound No. 86);

Mass (m/z): 491 (M⁺+1).
3-[3-{[1-(Cyclopropylcarbonyl)piperidin-3-yl]oxy}-4-(difluoromethoxy)phenyl]-1,7-dioxa-2-azaspiro[4.4]non-2-ene (Compound No. 87);

Mass (m/z); 437 (M⁺+1).
3-[3-{[1-(Cyclopentylcarbonyl)piperidin-3-yl]oxy}-4-(difluoromethoxy)phenyl]-1,7-dioxa-2-azaspiro[4.4]non-2-ene (Compound No. 88);

Mass (m/z): 465 (M⁺+1).
3-[4-(Difluoromethoxy)-3-{[1-(ethylsulfonyl)piperidin-3-yl]oxy}phenyl]-1,7-dioxa-2-azaspiro[4.4]non-2-ene (Compound No. 89);

Mass (m/z): 461 (M⁺+1).
3-[3-{[(3S)-1-acetylpyrrolidin-3-yl]oxy}-4-(difluoromethoxy)phenyl]-1,7-dioxa-2-azaspiro[4.4]non-2-one (Compound No. 93);

Mass (m/z): 397.1
3-[4-(Difluoromethoxy)-3-{[1-(phenylcarbonyl)piperidin-4-yl]oxy}phenyl]-1,7-dioxa-2-azaspiro[4.4]non-2-ene (Compound No. 95);

Mass (m/z): 473 (M⁺+1).
3-[4-(Difluoromethoxy)-3-{[1-(morpholin-4-ylcarbonyl)piperidin-4-yl]oxy}phenyl]-1,7-dioxa-2-azaspiro[4.4]non-2-ene (Compound No. 96);

Mass (m/z): 482 (M⁺+1).
3-[4-(Difluoromethoxy)-3-{[1-(phenylcarbonyl)piperidin-3-yl]oxy}phenyl]-1,7-dioxa-2-azaspiro[4.4]non-2-ene (Compound No. 97);

Mass (m/z); 473 (M⁺+1).
3-[4-(Difluoromethoxy)-3-{[1-(morpholin-4-ylcarbonyl)piperidin-3-yl]oxy}phenyl]-1,7-dioxa-2-azaspiro[4.4]non-2-ene (Compound No. 98);

Mass (m/z): 482 (M⁺+1).

3-[4-(Difluoromethoxy)-3-({1-[(trifluoromethyl)sulfonyl]piperidin-3-yl}oxy)phenyl]-1,7-dioxa-2-aza spiro[4.4]non-2-ene (Compound No. 99);

Mass (m/z): 501 (M⁺+1).
3-[4-(Difluoromethoxy)-3-{[(2R)-1-(phenylcarbonyl)pyrrolidin-2-yl]methoxy}phenyl]-1,7-dioxa-2-azaspiro[4.4]non-2-ene (Compound No. 100);

Mass (m/z): 473.14 (M⁺+1).
3-[3-{[(2R)-1-acetylpyrrolidin-2-yl]methoxy}-4-(difluoromethoxy)phenyl]-1,7-dioxa-2-azaspiro[4.4]non-2-one (Compound No. 101);

Mass (m/z): 411.25 (M⁺+1).
3-[4-(Difluoromethoxy)-3-{[(2R)-1-propanoylpyrrolidin-2-yl]methoxy}phenyl]-1,7-dioxa-2-azaspiro[4.4]non-2-one (Compound No. 102);

Mass (m/z): 425.27 (M⁺+1).
3-[3-{[(2R)-1-(cyclopropylcarbonyl)pyrrolidin-2-yl]methoxy}-4-(difluoromethoxy)phenyl]-1,7-dioxa-2-azaspiro[4.4]non-2-ene (Compound No. 103);

Mass (m/z): 437.28 (M⁺+1).
3-[3-{[(3S)-1-(cyclopropylcarbonyl)pyrrolidin-3-yl]oxy}-4-(difluoromethoxy)phenyl]-1,7-dioxa-2-azaspiro[4.4]non-2-ene (Compound No. 104);

Mass (m/z): 423.27 (M⁺+1).
3-[3-{[(3S)-1-(cyclopentylcarbonyl)pyrrolidin-3-yl]oxy}-4-(difluoromethoxy)phenyl]-1,7-dioxa-2-azaspiro[4.4]non-2-ene (Compound No. 105);

Mass (m/z): 451.30 (M⁺+1).
3-[4-(Difluoromethoxy)-3-({(3R)-1-[(4-fluorophenyl)carbonyl]pyrrolidin-3-yl}-oxy)phenyl]-1,7-dioxa-2-azaspiro[4.4]non-2-ene (Compound No. 106);

Mass (m/z): 491.29 (M⁺+1).

### Scheme V

The following compounds of Formula XXV b were prepared by following the procedures described in WO 2005/021515,
{3-[4-(Difluoromethoxy)-3-methoxyphenyl]-4,5-dihydroisoxazole-5,5-diyl}dimethanol (Compound No. 107);

Mass (m/z): 304.31 (M⁺+1).
3-[4-(Difluoromethoxy)-3-methoxyphenyl]-1,8-dioxa-2-azaspiro[4.5]dec-2-ene (Compound No. 111);

Mass (m/z): 314.26 (M⁺+1).
7-[4-(Difluoromethoxy)-3-methoxyphenyl]-5-oxa-6-azaspiro[3.4]oct-6-ene (Compound No. 115);

Mass (m/z): 284.03 (M⁺+1).
3-[4-(Difluoromethoxy)-3-methoxyphenyl]-1-oxa-2-azaspiro[4.4]non-2-ene (Compound No. 116);

Mass (m/z): 298.08 (M⁺+1).
3-[4-(Difluoromethoxy)-3-methoxyphenyl]-1-oxa-2-azaspiro[4.5]dec-2-ene (Compound No. 117);

Mass (m/z): 312.06 (M⁺+1).

### Scheme VI

### Synthesis of a Compound of Formula XXVIII

### Step a: Formula XXVII

To a solution of a compound of Formula XXVI (0.00078 mole) in dichloromethane (10 mL), triethylamine (0.003 mole) was added and the reaction mixture was cooled to 0° C. Methane sulphonyl chloride (0.0023 mole) was added drop wise and the reaction mixture was stirred at 0° C. to room temperature for about 2 hours. The reaction mixture was then diluted with dichloromethane and washed with sodium bicarbonate solution. Organic layer was washed with water and saturated sodium chloride solution, dried over anhydrous sodium sulphate and concentrated under reduced pressure to furnish the crude title compound.

### Step b: Formula XXVIII

To a solution of a compound of Formula XXVII (0.00076 mole) in dimethylformamide (5 mL), sodium sulphide. 9H₂O (0.0019 mole) was added and the reaction mixture was refluxed at 90-100° C. for about 14-16 hours. Excess of the solvent was evaporated, water was added to the residue and the solution was extracted with ethyl acetate, dried over anhydrous sodium sulphate and concentrated under reduced pressure to furnish the pure title compound.

The following compounds were prepared by following the above procedure 3-[4-(Difluoromethoxy)-3-methoxyphenyl]-1-oxa-7-thia-2-azaspiro[4.4]non-2-ene (Compound No. 108);

Mass (m/z): 316.24 (M⁺+1).
7-[4-(Difluoromethoxy)-3-methoxyphenyl]-5-oxa-2-thia-6-azaspiro[3.4]oct-6-ene (Compound No. 110);

Mass (m/z): 302.17 (M⁺+1).

### Synthesis of a Compound of Formula XXIX

A compound of Formula XXVIII (0.00015 mole) was dissolved in methanol (5 mL), sodium periodate (0.00015 mole) was added at 0° C. and the reaction mixture was stirred at room temperature for about 5 hours. The residue was filtered and the organic solvent was removed under reduced pressure to furnish solid compound, which was further purified by preparative TLC using 50% ethyl acetate in hexane.

The following compounds were prepared by following the above procedure
3-[4-(Difluoromethoxy)-3-methoxyphenyl]-7-hydroxy-1-oxa-7-thionia-2-azaspiro[4.4]non-2-ene (Compound No. 109);

Mass (m/z): 332.24 (M⁺+1).
7-[4-(Difluoromethoxy)-3-methoxyphenyl]-2-hydroxy-5-oxa-2-thionia-6-azaspiro[3.4]oct-6-ene (Compound No. 113);

Mass (m/z): 317.98 (M⁺+1).

### Synthesis of a Compound of Formula XXX

To a solution of a compound of Formula XXVIII (0.00022 mole) in dichloromethane (5 mL), m-chloroperoxy benzoic acid (0.00033 mole) was added at 0° C. and the reaction mixture was stirred at room temperature over-night. The reaction mixture was extracted with water. Organic layer was washed with 1N sodium hydroxide solution and then with water, dried over sodium sulphate and concentrated under reduced pressure to furnish the crude title compound, which was further purified by preparative TLC using ethyl acetate as eluent.

The following compounds were prepared by following the above procedure
3-[4-(Difluoromethoxy)-3-methoxyphenyl]-1-oxa-7-thia-2-azaspiro[4.4]non-2-ene 7,7-dioxide (Compound No. 114)

Mass (m/z): 347.92 (M⁺+1)
7-[4-(Difluoromethoxy)-3-methoxyphenyl]-5-oxa-2-thia-6-azaspiro[3,4]oct-6-ene 2,2-dioxide (Compound No. 120)

Mass (m/z): 333.92 (M⁺+1)

### Scheme VII

### Synthesis of a Compound of Formula XXXIV

### Step a: Formula XXXII

The compounds of Formula XXXII were prepared by following the procedures described in WO 2005/021515.

### Step b: Formula XXXIII

A compound of Formula XXXII (0.00071 mole) was taken in tetrahydrofuran (15 mL). Methanol (5 mL) was added to it. Sodium borohydride (0.0014 mole) was added and the reaction mixture was stirred at room temperature over-night. The reaction was quenched with saturated ammonium chloride and the solvent was removed under reduced pressure. Water was added to the residue and extraction was done with ethyl acetate, the residue was dried over sodium sulphate and concentrated under reduced pressure to furnish the crude title compound, which was further purified by column chromatography using silica gel (100-200).

### Step c: Formula XXXIV

A compound of Formula XXXIII (0.00027 mole) was dissolved in a mixture of ethanol:water (10:2 mL), potassium hydroxide (0.0005 mole) was added and the reaction mixture was stirred at refluxing temperature over-night. Excess solvent was removed under reduced pressure. Water was added to the residue and it was extracted with ethyl acetate, washed with saturated sodium chloride solution, dried over sodium sulphate and concentrated under reduced pressure. The compound was purified by column chromatography.

The following compound was prepared by following the above procedure
7-[4-(Difluoromethoxy)-3-methoxyphenyl]-2,5-dioxa-6-azaspiro[3.4]oct-6-ene (Compound No. 123)

Mass (m/z): 286.02 (M⁺+1)

### Scheme VIII

### Synthesis of a Compound of Formula XXXVII

### Step a: Formula XXXV a

Cyclopentane 1,3-diol (0.0014 mole) and tert-butyl dimethyl silyl chloride (0.0008 mole) in dichloromethane (5 mL) were treated dropwise with 1,8-diaza bicyclo [5.4.0]undec-7-ene (0.0008 mole) at room temperature and the reaction mixture was stirred for about 14 hours. The reaction mixture was then diluted with dichloromethane, washed with 1 N HCl solution, followed by saturated sodium bicarbonate solution and brine solution. It was dried over anhydrous sodium sulphate and concentrated under reduced pressure to furnish the title compound.

### Step b: Formula XXXV

It was prepared by following Scheme I.

### Step c: Formula XXXVI

A compound of Formula XXXV (0.00035 mole), a compound of Formula XXXV a (0.00035 mole) and triphenyl phosphine (0.00052 mole) were taken together in tetrahydrofuran (10 mL) and the reaction mixture was stirred for about 10 minutes, followed by dropwise addition of diisopropyl azodicarboxylate (0.00052 mole). The reaction mixture was stirred over-night, solvent was then removed under reduced pressure and the residue purified by column chromatography.

### Step d: Formula XXXVII

A compound of Formula XXXVI (70 mg) was taken in ethanolic HCl (10 mL) and stirred over-night. Ethanol was removed under reduced pressure, water was added and the solution was extracted with ethyl acetate. It was washed with saturated sodium chloride solution, dried over anhydrous sodium sulphate and concentrated under reduced pressure. Purification was done by preparative TLC using ethyl acetate:hexane (1:1) to get the pure title compound.

The following compound was prepared by following the above procedure
3-[2-(Difluoromethoxy)-5-(1,7-dioxa-2-azaspiro[4.4]non-2-en-3-yl)phenoxy]cyclopentanol (Compound No. 129);

Mass (m/z): 370.13 (M⁺+1).

### Scheme IX

### Synthesis of a Compound of Formula XXXIX

A compound of Formula XXXV (0.701 mmole), copper II acetate (0.701 mmole), 4-(n-butoxycarbonyl)aminophenyl boronic acid (1.4 mmole), 4 A° molecular sieves were taken together in dichloromethane. Tri ethyl amine (3.505 mmole) was added to the reaction mixture and stirred together at room temperature over-night. The reaction mixture was then filtered through celite pad. The organic solvent was evaporated under reduced pressure, diluted with ethyl acetate, washed with saturated sodium bicarbonate solution followed by brine solution, dried over anhydrous sodium sulphate and concentrated under reduced pressure. The crude mixture was purified by column chromatography.

The following compounds were prepared by following the above procedure
tert-butyl {4-[2-(Difluoromethoxy)-5-(1,7-dioxa-2-azaspiro[4.4]non-2-en-3-yl)phenoxy]phenyl}carbamate (Compound No. 125);

Mass (m/z): 477.07 (M⁺+1).
3-[4-(Difluoromethoxy)-3-(4-fluorophenoxy)phenyl]-1,7-dioxa-2-azaspiro[4.4]non-2-ene (Compound No. 131);

Mass (m/z): 380.09 (M⁺+1).
3-[3-(4-Chlorophenoxy)-4-(difluoromethoxy)phenyl]-1,7-dioxa-2-azaspiro[4.4]non-2-ene (Compound No. 132);

Mass (m/z): 396.11 (M⁺+1).
3-{4-(Difluoromethoxy)-3-[4-(trifluoromethoxy)phenoxy]phenyl}-1,7-dioxa-2-azaspiro[4.4]non-2-ene (Compound No. 133);

Mass (m/z): 446.14 (M⁺+1).
3-{4-(Difluoromethoxy)-3-[4-(trifluoromethyl)phenoxy]phenyl}-1,7-dioxa-2-azaspiro[4.4]non-2-ene (Compound No. 134);

Mass (m/z): 430.18 (M⁺+1).

### Synthesis of a Compound of Formula XL

Tert-butyl {4-[2-(difluoromethoxy)-5-(1,7-dioxa-2-azaspiro[4.4]non-2-en-3-yl)phenoxy]phenyl}carbamate (Compound No. 125) (50 mg) was dissolved in ethereal HCl and the mixture was stirred at room temperature over-night. The solvent was evaporated under reduced pressure to obtain a solid, which was washed with hexane and dried.

The following compound was prepared by following the above procedure Hydrochloride salt of 4-[2-(difluoromethoxy)-5-(1,7-dioxa-2-azaspiro[4.4]non-2-en-3-yl)phenoxy]aniline (Compound No. 124);

Mass (m/z): 377.0 (M⁺+1).

### Synthesis of a Compound of Formula XLI

Hydrochloride salt of 4-[2-(difluoromethoxy)-5-(1,7-dioxa-2-azaspiro[4.4]non-2-en-3-yl)phenoxy]aniline (Compound No. 124) (80 mg, 0.212 mmole) and methane sulphonyl chloride (0.424 mmole) were taken in pyridine (1 mL) and the reaction mixture was stirred at room temperature over-night. The solvent was evaporated under reduced pressure. Water was added to the residue and extracted with ethyl acetate, dried over anhydrous sodium sulphate and concentrated under reduced pressure to furnish the compound, which was further purified by preparative TLC.

The following compound was prepared by following the above procedure {4-[2-(Difluoromethoxy)-5-(1,7-dioxa-2-azaspiro[4.4]non-2-en-3-yl)phenoxy]phenyl}methane sulfonamide (Compound No. 136);

Mass (m/z): 455.10 (M⁺+1).

### Synthesis of a Compound of Formula XLII

To the solution of hydrochloride salt of 4-[2-(difluoromethoxy)-5-(1,7-dioxa-2-azaspiro[4.4]non-2-en-3-yl)phenoxy]aniline (Compound No. 124) (80 mg, 0.212 mmole) in dichloromethane (2 mL) triethyl amine (0.425 mmole) and acetic anhydride (0.425 mmole) were added and the reaction mixture was stirred at room temperature over-night. Water was added to the reaction mixture and extracted with dichloromethane, washed with brine solution, dried over anhydrous sodium sulphate and concentrated under reduced pressure to furnish crude title compound with was purified by preparative TLC.

The following compound was prepared by following the above procedure
N-{4-[2-(Difluoromethoxy)-5-(1,7-dioxa-2-azaspiro[4.4]non-2-en-3-yl)phenoxy]phenyl}acetamide (Compound No. 135);

Mass (m/z): 419.13 (M⁺+1).

### Scheme X

### Synthesis of Compound of Formula XLIV

A compound of Formula XXXV (0.350 mmole) and bromo benzene (0.636 mimic) were taken in pyridine (2.5 mL), potassium carbonate (0.507 mmole) was added and the reaction mixture was stirred at 150° C. temperature for about 5 minutes. Cu powder (0.314 mmole) was added and the mixture was stirred again at 150° C. temperature for about 24 hours, It was neutralized with HO, water was added, extraction was done with ethyl acetate, washings were done with brine solution, dried over anhydrous sodium sulphate and concentrated under reduced pressure to furnish a crude mixture which was purified by preparative TLC using 30% ethyl acetate in hexane as solvent.

The following compound was prepared by following the above procedure
3-[4-(Difluoromethoxy)-3-phenoxyphenyl]-1,7-dioxa-2-azaspiro[4.4]non-2-ene (Compound No. 112);

Mass (m/z): 361.96 (M⁺+1).

### Synthesis of a Compound of Formula XLVI

To a solution of a compound of Formula XXXV (0.526 mmole) in dimethyl sulphoxide (3 mL), suspension of potassium fluoride in dimethyl sulphoxide (1.052 mmole) was added, followed by cesium carbonate (2.104 mmole) and stirred for about 10 minutes. 4-Bromo pyridine (0.784 mmole) was added and the reaction mixture was refluxed at 140° C. for about 6 hours. Water was added and the mixture was extracted with ethyl acetate, washed with brine solution, dried over anhydrous sodium sulphate and concentrated under reduced pressure. The crude compound was purified by column chromatography.

The following compound was prepared by following the above procedure
3-[4-(Difluoromethoxy)-3-(pyridin-4-yloxy)phenyl]-1,7-dioxa-2-azaspiro[4.4]non-2-ene (Compound No. 137);

Mass (m/z): 362.92 (M⁺+1).

### Scheme XI

### Synthesis of a Compound of Formula XLVIII

The following compound of Formula XLVIII was prepared by following the procedures described in WO 2005/021515.
3-[4-(Difluoromethoxy)-3-methoxyphenyl]-1,9,12-trioxa-2-azadispiro[4.2.4.2]tetradec-2-cue (Compound No. 118);

Mass (m/z): 369.99 (M⁺+1).

### Synthesis of a Compound of Formula XLIX

To the solution of a compound of Formula XLVIII (0.271 mmole) in dichloromethane (2 mL), trifluoroacetic acid (1.355 mmole) was added drop wise in about 1 hour. Water (0.1 mL) was added and the reaction mixture was stirred vigorously for about 6 hours. The reaction mixture was diluted with dichloromethane, washed with sodium bicarbonate solution, the organic layer was dried over anhydrous sodium sulphate and concentrated under reduced pressure. The crude compound was purified by preparative TLC.

The following compound was prepared by following the above procedure
3-[4-(Difluoromethoxy)-3-methoxyphenyl]-1-oxa-2-azaspiro[4.5]dec-2-en-8-one (Compound No. 119);

Mass (m/z): 326.0 (M⁺+1).

### Synthesis of a Compound of Formula L

To the solution of 3-[4-(difluoromethoxy)-3-methoxyphenyl]-1-oxa-2-azaspiro[4,5]dec-2-en-8-one (Compound No. 119) (200 mg, 0.615 mmole) in methanol:tetrahydrofuran (0.2:5.0 mL), sodium borohydride (46 mg, 1.230 mmole) was added. The reaction mixture was stirred for about 3 hours and quenched with ammonium chloride solution. Excess of solvent was removed and the mixture was extracted with ethyl acetate, washed with brine solution, dried over anhydrous sodium sulphate and concentrated under reduced pressure. The crude compound obtained was purified by preparative TLC.

The following compound was prepared by following the above procedure
3-[4-(Difluoromethoxy)-3-methoxyphenyl]-1-oxa-2-azaspiro[4.5]dec-2-en-8-ol (Compound No. 122);

Mass (m/z): 327.98 (M⁺+1).

### Synthesis of a Compound of Formula LI

3-[4-(Difluoromethoxy)-3-methoxyphenyl]-1-oxa-2-azaspiro[4.5]dec-2-en-8-one (Compound No. 119) (250 mg, 0.769 mmole), hydroxylamine hydrochloride (106 mg, 1.538 mmole) and potassium carbonate (530 mg, 3.845 mmole) were taken together in acetonitrile (3 mL). The reaction mixture was stirred for about 6 hours at room temperature. Excess of solvent was removed under reduced pressure and water was added to the residue, Solid, which separated out was filtered and dried under vacuo.

The following compound was prepared by following the above procedure
3-[4-(Difluoromethoxy)-3-methoxyphenyl]-1-oxa-2-azaspiro[4.5]dec-2-en-8-one oxime (Compound No. 121);

Mass (m/z): 340.99 (M⁺+1).

### Example 1

### Biological Assay

### PDE-4 Enzyme Assay

The efficacy of compounds of PDE-4 inhibitors was determined by an enzyme assay using cell lysate of HEK293 cells transfected with PDE4B2 or PDE7A1 plasmids as PDE4B or PDE7A source. Some compounds were screened against PDE7A, enzyme. The enzyme reaction was carried out in the presence of cAMP (1 μM) at 30° C. in the presence or absence of test compound for 45-60 min. An aliquot of this reaction mixture was taken further for the ELISA assay and the protocol of the kit followed to determine level of cAMP in the sample. The concentration of the cAMP in the sample directly correlates with the degree of PDE-4 or PDE-7 enzyme inhibition. Results were expressed as percent control and the IC₅₀ values of test compounds were reported. IC₅₀ values of test compounds were found to be in the range from about 10 μM to about 1 nM concentration.

### Cell Based Assay for TNF-α Release

### Method of Isolation of Human Peripheral Blood Mononuclear Cells:

Human whole blood was collected in vacutainer tubes containing heparin or EDTA as an anti coagulant. The blood was diluted (1:1) in sterile phosphate buffered saline and 10 mL was carefully: layered over 5 mL Ficoll Hypaque gradient (density 1.077 gimp in a 15 mL conical centrifuge tube. The sample was centrifuged at 3000 rpm for 25 minutes in a swing-out rotor at room temperature. After centrifugation, interface of cells were collected, diluted at least 1:5 with PBS (phosphate buffered saline) and washed three times by centrifugation at 2500 rpm for 10 minutes at room temperature. The cells were resuspended in serum free RPMI 1640 medium at a concentration of 2 million cells/mL.

### LPS Stimulation of Human PBMNC's:

PBMN cells (0.1 mL; 2 million/mL) were co-incubated with 20 mL of compound (final DMSO concentration of 0.2%) for 10 min in a flat bottom 96 well microtiter plate. Compounds were dissolved in DMSO initially and diluted in medium for a final concentration of 0.2 DMSO. LPS (1 mg/mL, final concentration) was then added at a volume of 10 μl per well. After 30 min, 20 μl of fetal calf serum (final concentration of 10%) was added to each well. Cultures were incubated overnight at 37° C. in an atmosphere of 5% CO₂ and 95% air. Supernatant were then removed and tested by ELISA for TNF-α release using a commercial kit (e.g. BD Biosciences). The level of TNF-α in treated wells was compared with the vehicle treated controls and inhibitory potency of a compound was expressed as IC₅₀ values calculated by using Graph pad prism. IC₅₀ values of some of the compounds was found to be in the range from about 10 μM to about 100 nM concentration.

$$Percent inhibition = 100 - \frac{Percent TNF \text{-} α drug treated}{Percent TNF \text{-} α in vehicle treated} \times 100$$

In-vitro Assay to Evaluate Efficacy of PDE4 Inhibitors in Combination with p38 MAP Kinase Inhibitors or Corticosteroids

The procedure was same as above except that the test compounds were added either singly or in combination with other therapeutic agents at sub-optimal doses. A synergistic effect was seen with the combination of PDE4 inhibitor with corticosteroid or PDE4 inhibitor with p38 MAP kinase inhibitor as compared to the individual compounds when used singly.

In-vitro Assay to Evaluate Efficacy of PDE4 Inhibitors in Combination with β2-Agonists
Measurement of Intracellular cAMP Elevation in U937 Cells

U937 cells (human promonocytic cell line) are grown in endotoxin-free RPMI 1640+HEPES medium containing 10% (v/v) heat-inactivated foetal bovine serum and 1% (v/v) of an antibiotic solution (5000 IU/mL penicillin, 5000 μg/mL streptomycin). Cells (0.25×10⁶/200 μl) are resuspended in Krebs' buffer solution and incubated at 37° C. for 15 min in the presence of test compounds or vehicle (20 μl). cAMP generation is initiated by adding 50 μl of 10 μM prostaglandin (PGE2). Reaction is stopped after 15 min, by adding 1 N HCl (50 μl) and assay mixture placed on ice for 30 min. Sample is centrifuged (450 g, 3 min), and levels of cAMP measured in the supernatant using cAMP enzyme-linked immunosorbent assay kit (Assay Designs). Percent inhibition is calculated by the following formula and IC₅₀ value determined using Graph pad prism.

$$Percent inhibition = 100 - \frac{\begin{matrix}Percent conversion \\ in drug treated\end{matrix}}{\begin{matrix}Percent conversion \\ in vehicle treated\end{matrix}} \times 100$$

In-vitro Functional Assay to Evaluate Efficacy of PDE4 Inhibitors in Combination with Muscarinic Receptor Antagonists

### Animals and Anaesthesia

Procure Guinea Pig (400-600 gm) from experimental animal facility at Ranbaxy Research laboratories. Remove trachea under anesthesia (sodium pentobarbital, 300 mg/kg i.p) and immediately keep it in ice-cold Krebs Henseleit buffer. Indomethacin (10 uM) is present throughout the KH buffer to prevent the formation of bronchoactive prostanoids.

### Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 mL Krebs Henseleit buffer maintained at 37° C. and bubbled with carbogen, at a basal tension of 1 gm. Change the buffer 4-5 times for about an hour. Equilibrate the tissue for 1 hr for stabilization. After 1 hr, challenge the tissue with 1 uM carbachol. Repeat this after every 2-3 washes till two similar consequetive responses are obtained. At the end of stabilization, wash the tissues for 30 minutes followed by incubation with suboptimal dose of MRA/Vehicle for 20 minutes prior to contraction of the tissues with 1 μM carbachol and subsequently assess the relaxant activity of the PDE4 inhibitor [10⁻⁹ M to 10⁻⁴ M] on the stabilized developed tension/response. Record the contractile response of tissues either on Powerlab data acquisition system or on Grass polygraph (Model 7). Express the relaxation as percentage of maximum carbachol response. Express the data as mean±s.e. mean for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

In-vitro Functional Assay to Evaluate Efficacy of PDE4 Inhibitors in Combination with Beta-Agonists

### Experimental Procedure:

Experimental Procedure and data analysis was the same as above except that tissues were stabilized with 1 μM carbachol or 10 μM histamine, washed for 30 minutes followed by a precontraction with histamine (10 μM) or carbachol (1 μM), Tension was allowed to develop to stabilize for 15-20 minutes followed by the cumulative addition of beta-agonists prior to incubation with suboptimal dose of PDE4 inhibitor. A potentiation of the relaxant activity of a beta agonist was seen with the addition of a PDE4 inhibitor.

### In-vivo Assay to Evaluate Efficacy of PDE4 Inhibitors in Combination MRA Compounds

### LPS Induced Airway Hyper-Reactivity in Rats

### Drug Treatment:

PDE-4 inhibitor and muscarinic receptor antagonist were instilled intratracheally under anesthesia at different doses, either alone or in combination.

### Method

Wistar rats (250-350 gm) were placed in body box of a whole body plethysmograph (Buxco Electronics, USA) to induce bronchoconstriction. Animals were allowed to acclimatize in the body box and were given successive challenges, each of 2 min duration, with PBS (vehicle thr acetylcholine) or acetylcholine (i.e. 24, 48, 96, 144, 384, and 768 mg/mL). The respiratory parameters were recorded online using Biosystem XA software, (Buxco Electronics, USA.) for 3 min. A gap of 2 min was allowed for the animals to recover and then challenged with the next higher dose of acetylcholine (ACh). This step was repeated until Penh of rats attained 2 times the value (PC-100) seen with PBS challenge. Following PBS/ACh challenge, Penh values (index of airway resistance) in each rat was obtained in the presence of PBS and different doses of ACh. Penh, at any chosen dose of Ach, was expressed as percent of PBS response. The Penh values thus calculated were fed into Graph Pad Prism software (Graphpad Software Inc., USA) and using a nonlinear regression analysis PC100 (2 folds of PBS value) values computed. Percent inhibition was computed using the following formula.

$$\% Inhibition = \frac{PC {100}_{TEST} - PC {100}_{CON}}{768 - PC {100}_{CON}} \times 100$$

where,
PC100_{CON}=PC100 in vehicle treated group
PC100_{TEST}=PC100 in group treated with a given dose of test compound
768=is the maximum amount of acetylcholine used

A synergistic effect was seen with the combination of selected PDE4 inhibitors with a MRA compound as compared to the individual compounds when used singly.

In-vivo Assay to Evaluate Efficacy of PDE4 Inhibitors in Combination with Corticosteroids

### LPS Induced Rat Neutrophilia Model

### Drug Treatment:

PDE-4 inhibitor and corticosteroids were instilled intratracheally under anesthesia at different doses, either alone or in combination

LPS challenge: One hour after drug instillation, (LPS 20 μg/200 μl of PBS) was instilled intratracheally. One group of vehicle treated rats were instilled with 200 μl of phosphate buffered saline (PBS) and served as negative control.
Bronchoalveolar lavage (BAL): Two hours after LPS challenge, bronchoalveolar lavage was performed; the animals were sacrificed using thiopentone sodium (150 mg/kg/i.p.). Trachea was cannulated and BAL was performed using Hank's Buffer salt solution (HBSS) (5 mL×10 times). The bronchoalveolar lavage fluid was centrifuged at 800 g for 5 min, at 4° C. and the pellet was resuspended in 1 mL HBSS. Total leukocyte count was performed in the resuspended sample by using hemocytometer. A cytocentrifuge preparation was made using the resuspended bronchoalveolar lavage fluid on a glass slide, stained with Leishmann's stain and then differential leukocyte counts were performed for computation of neutrophil. Statistical significance of each parameter in different treatment groups was determined with respect to vehicle control group using one-way analysis of variance followed by Dunnett's ‘t’ test for multiple comparison. A p level of ≦0.05 was considered to be statistically significant. ED₅₀ value was obtained by regression analysis of concentration and percent inhibition data using GraphPad Prism software v4.2. Percent inhibition was computed using the following formula.

$$\% Inhibition = \frac{{Neu}_{LPS} - {Neu}_{TEST}}{{Neu}_{LPS} - {Neu}_{PBS}} \times 100$$

where,
Neu_{LPS}=Neutrophil count in vehicle treated LPS challenged group
Neu_{TEST}=Neutrophil count in group treated with a given dose of test compound
Neu_{PBS}=Percentage of Neutrophil in group challenged with PBS

A synergistic effect was seen with the combination of selected PDE4 inhibitors with a corticosteroid as compared to the individual compounds when used singly

We claim:

1. A compound having the structure of Formula I: 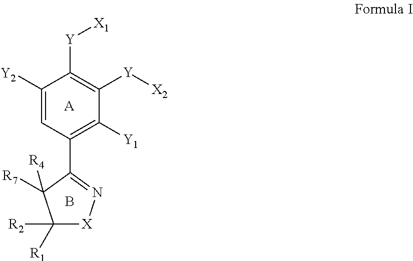
its pharmaceutically acceptable salts, pharmaceutically acceptable solvates, enantiomers, diastereomers, polymorphs or N-oxides wherein
when X is oxygen,
R₁ is hydrogen, alkyl, heterocyclyl, —(CH₂)₁₋₄OR′, provided that R₂ is also (CH₂)₁₋₄ OR′ (wherein R′ is hydrogen, alkyl, alkenyl, alkynyl, (un)saturated cycloalkyl, aryl, heterocyclyl or heteroaryl), —C(═O)NRₓR_{y} provided that R₂ is also —C(O)NRₓR_{y} [wherein Rₓ and R_{y} are hydrogen, alkyl, alkenyl of three to six carbon atoms, alkynyl of three to six carbon atoms, cycloalkyl, —SO₂R₅ (wherein R₅ is hydrogen, alkyl, alkenyl, alkynyl, aryl, cycloalkyl, alkaryl, heteroaryl, heteroarylalkyl, heterocyclyl or heterocyclylalkyl), aryl, alkaryl, heteroaryl, heterocyclyl, heteroarylalkyl, and heterocyclylalkyl], —(CH₂)ₘ—C(═O)R₃ (wherein m is an integer in the range of 0-2 and R₃ is cycloalkyl, aryl, optionally substituted Rₚ or R_{q}, wherein Rₚ is heterocyclyl or heteroaryl ring wherein the said rings are attached to (CH₂)ₘC(═O) through N, and R_{q} is heterocyclyl or heteroaryl ring wherein the said rings are attached to —(CH₂)ₘC(═O) through C);
R₂ is —(CH₂)ₘC(═O)R₃ (wherein m and R₃ are the same as defined earlier), —(CH₂)₁₋₄OR′, provided R₁ is also (CH₂)₁₋₄OR′ (wherein R′ is same as defined earlier); —C(═O)NRₓR_{y} provided R₁ is also —C(═O)NRₓR_{y} (wherein Rₓ and R_{y} are same as defined earlier), or R₁ and R₂ may together form optionally substituted cycloalkyl or heterocyclyl ring wherein the substituents of such a joint R₁-R₂ ring(s) can be oxo, alkyl, alkenyl, alkynyl, halogen (F, Cl, Br, I), nitro, —NH₂, ═NOH, —C(═O)NRₓR_{y}, —COORₓ, —COONRₓR_{y} (wherein Rₓ and R_{y} are the same as defined earlier), —NHCOOR₆ (wherein R₆ is alkyl, alkenyl, alkynyl, cycloalkyl, alkaryl, heteroarylalkyl or heterocyclylalkyl), cyano, hydroxy, alkoxy, or substituted amino;
R₄ is hydrogen, alkyl, —OR₅ (wherein R₅ is the same as defined earlier), halogen (F, Cl, Br, I), —NH₂, substituted amino, cyano, carboxy, or —C(═O)NRₓR_{y} (wherein Rₓ and R_{y} are the same as defined above), or R₂ and R₄ may together form optionally substituted 4-12 membered (un)saturated monocyclic or bicyclic ring system fused to ring B having 0-4 heteroatom(s) selected from N, O and S with the proviso that R₂ and R₄ together does not form —CH₂—O—CH₂—O—CH₂—, wherein the substituents can be one or more of alkyl, halogen (F, Cl, Br, D, hydroxy, alkoxy, —NH₂ or substituted ammo;
R₇ is hydrogen, alkyl, alkenyl, alkynyl, —OR₅ (wherein R₅ is the same as defined earlier), halogen (F, Cl, Br, I), cyano, —NH₂ or substituted amino;
X₁ and X₂ are hydrogen, alkyl, alkaryl, cycloalkyl, heterocyclyl, heteroaryl, aryl, heteroarylalkyl or heterocyclylalkyl, —(CH₂)ₘCOR₃, —(CH₂)₅C(═O)NRₓR_{y} or —(CH₂)_{g1}C(═O)OR₃ (wherein g and g₁ are an integer from 0-3, m Rₓ, R_{y} and R₃ are same as defined earlier);
Y is an oxygen atom, a sulphur atom, or —NR (wherein R is hydrogen, acyl, aryl, or alkyl);
Y₁ and Y₂ are independently selected from hydrogen, alkyl, —OR (wherein R is the same as defined earlier), —SR (wherein R is the same as defined earlier, and —NHR (wherein R is the same as defined earlier);
Any of Y₁ and X₂ & X₁ and Y₂ may together form a cyclic ring fused with the ring A shown in Formula I, the ring containing 3-5 carbon atoms within the ring and having 1-3 heteroatoms such as N, O and S.
X₁ and X₂ may together form a cyclic ring fused with the ring A shown in Formula I, the ring containing 3-5 carbon atoms within the ring and having 2-3 heteroatoms such as N, O and S.
When X is NR₇ or S, wherein R₇ is hydrogen or lower alkyl (C₁-C₆)
R₁ and R₂ are independently alkyl, alkenyl, alkynyl, alkoxy, hydroxyl, cyano, nitro, halogen (F, Cl, Br, I), heteroaryl, heterocyclyl, heteroarylalkyl, heterocyclylalkyl, substituted amino, carboxy, —(CH₂)ₘ(C═O)R₃ (wherein in and R₃ are the same as defined earlier), —C(═O)NRₓR_{y} (wherein Rₓ and R_{y} are the same as defined above), or —(CH₂)₁₋₄OR′ (wherein R′ is same as defined earlier) or R₁ and R₂ may together form optionally substituted cycloalkyl or heterocyclyl ring wherein the substituents of such a joint R₁-R₂ ring(s) can be oxo, alkyl, alkenyl, alkynyl, halogen (F, Cl, Br, I), nitro, —NH₂, —C(═O)NRₓR_{y}, —COORₓ, —COONRₓR_{y} (wherein Rₓ and R_{y} are the same as defined earlier), —NHCOOR₆ (wherein R₆ is alkyl, alkenyl, alkynyl, cycloalkyl, alkaryl, heteroarylalkyl or heterocyclylalkyl), cyano, hydroxy, alkoxy or substituted amino;
R₄ is hydrogen, alkyl, halogen (F, Cl, Br, I), —OR₅ (wherein R₅ is the same as defined earlier), cyano, carboxy, substituted amino, or —C(═O)NRₓR_{y} (wherein Rₓ and R_{y} are the same as defined above), or R₂ and R₄ may together form optionally substituted 4-12 membered (un)saturated monocyclic or bicyclic ring system used to ring B having 0-4 heteroatom(s) such as N, O and S, with the proviso that R₂ and R₄ together does not form —CH₂—O—CH₂—O—CH₂—, wherein the substituents can be one or more of alkyl, halogen (F, Cl, Br, I), hydroxy, alkoxy, or amino;
R₇ is hydrogen, alkyl, alkenyl, alkynyl, —OR₅ (wherein R₅ is the same as defined earlier), halogen (F, Cl, Br, I), cyano, —NH₂ or substituted amino;
X₁ and X₂ are alkyl, cycloalkyl, alkaryl, heteroaryl, heterocyclyl, heteroarylalkyl or heterocyclylalkyl, —(CH₂)_{g}C(═O)NRₓR_{y} or —(CH₂)_{g1}C(═O)OR₃ (wherein g, Rₓ, R_{y}, R₃ and g₁ are an integer from 0-3);
Y is an oxygen atom, a sulphur atom, or —NR (Wherein R is hydrogen, acyl, aryl or alkyl);
Y₁ and Y₂ are independently hydrogen, alkyl, —OR (wherein R is the same as defined earlier), —SR (wherein R is the same as defined earlier), or —NHR (wherein R is the same as defined earlier);
Any of Y₁ and X₂ & X₁ and Y₂ may together form a cyclic ring fused with the ring A shown in Formula I, the ring containing 3-5 carbon atoms within the ring and having 1-3 heteroatoms such as N, O and S;
X and X₂ may together form a cyclic ring fused with the ring A shown in Formula I, the ring containing 3-5 carbon atoms within the ring and having 2-3 heteroatoms such as N, O and S.

2. A compound which is selected from:
3-[3-{[(3S)-1-Benzylpyrrolidin-3-yl]oxy}-4-(difluoromethoxy)phenyl]-1,7-dioxa-2-azaspiro[4.4]non-2-ene (Compound No. 1);
3-[2-(Difluoromethoxy)-5-(1,7-dioxa-2-azaspiro[4.4]non-2-en-3-yl)phenoxy]propan-1-ol (Compound No. 2);
[2-(Difluoromethoxy)-5-(1,7-dioxa-2-azaspiro[4.4]non-2-en-3-yl)phenoxy]acetonitrile (Compound No. 3);
4-[(5S or 5R)-1,7-dioxa-2-azaspiro[4.4]non-2-en-3-yl]-2-methoxyphenol (Compound No. 4);
4-[(5R or 5S)-1,7-Dioxa-2-azaspiro[4.4]non-2-en-3-yl]-2-methoxyphenol (Compound No. 5);
5-[(5S or 5R)-1,7-Dioxa-2-azaspiro[4.4]non-2-en-3-yl]-2-methoxyphenol (Compound No. 6);
(5S or 5R)-3-(3,4-Dimethoxyphenyl)-1,7-dioxa-2-azaspiro[4.4]non-2-ene (Compound No. 7);
(5R or 5S)-3-(3,4-Dimethoxyphenyl)-1,7-dioxa-2-azaspiro[4.4]non-2-ene (Compound No. 8);
2-(Benzyloxy)-4-(1,7-dioxa-2-azaspiro[4.4]non-2-en-3-yl)phenol (Compound No. 9);
2-[2-(Difluoromethoxy)-5-(1,7-dioxa-2-azaspiro[4.4]non-2-en-3-yl)phenoxy]ethanol (Compound No. 10);
3-[4-(Difluoromethoxy)-3-ethoxyphenyl]-1,7-dioxa-2-azaspiro[4.4]non-2-ene (Compound No. 11);
3-[3-(Cyclohexyloxy)-4-(difluoromethoxy)phenyl]-1,7-dioxa-2-azaspiro[4.4]non-2-ene (Compound No. 12);
(5R or 5S)-3-[4-(Difluoromethoxy)-3-methoxyphenyl]-1,7-dioxa-2-azaspiro[4.4]non-2-ene (Compound No. 13);
(5S or 5R)-3-[4-(Difluoromethoxy)-3-methoxyphenyl]-1,7-dioxa-2-azaspiro[4.4]non-2-ene (Compound No. 14);
Ethyl [2-(difluoromethoxy)-5-(1,7-dioxa-2-azaspiro[4.4]non-2-en-3-yl)phenoxy]acetate (Compound No. 15);
3-[4-(Difluoromethoxy)-3-(2-morpholin-4-ylethoxy)phenyl]-1,7-dioxa-2-azaspiro[4.4]non-2-ene (Compound No. 16);
2-(Difluoromethoxy)-5-(1,7-dioxa-2-azaspiro[4.4]non-2-en-3-yl)phenyl cyclohexanecarboxylate (Compound No. 17);
5-[2-(Difluoromethoxy)-5-(1,7-dioxa-2-azaspiro[4.4]non-2-en-3-yl)phenoxy]pentanoic acid (Compound No. 18);
3-[3-(2,2,2-Trifluoroethoxy)-4-(difluoromethoxy)phenyl]-1,7-dioxa-2-azaspiro[4.4]non-2-one (Compound No. 19);
3-[3-(Cyclopentylmethoxy)-4-(difluoromethoxy)phenyl]-1,7-dioxa-2-azaspiro[4.4]non-2-one (Compound No. 20);
N-cyclopropyl-2-[2-(difluoromethoxy)-5-(1,7-dioxa-2-azaspiro[4.4]non-2-en-3-yl)phenoxy]acetamide (Compound No. 21);
2-[2-(Difluoromethoxy)-5-(1,7-dioxa-2-azaspiro[4.4]non-2-en-3-yl)phenoxy]acetamide (Compound No. 22);
2-[2-(Difluoromethoxy)-5-(1,7-dioxa-2-azaspiro[4.4]non-2-en-3-yl)phenoxy]-N-methylacetamide (Compound No. 23);
3-[3-(Cyclopentyloxy)-4-(2,2,2-trifluoroethoxy)phenyl]-1,7-dioxa-2-azaspiro[4.4]non-2-one (Compound No. 24);
2-(Difluoromethoxy)-5-(1,7-dioxa-2-azaspiro[4.4]non-2-en-3-yl)phenyl cyclopropanecarboxylate (Compound No. 25);
2-(Difluoromethoxy)-5-(1,7-dioxa-2-azaspiro[4.4]non-2-en-3-yl)phenyl morpholine-4-carboxylate (Compound No. 26);
2-(Difluoromethoxy)-5-(1,7-dioxa-2-azaspiro[4.4]non-2-en-3-yl)phenyl benzoate (Compound No. 27);
5-[2-(Difluoromethoxy)-5-(1,7-dioxa-2-azaspiro[4.4]non-2-en-3-yl)phenoxy]pentanamide (Compound No. 28);
3-[3-Propoxy-4-(2,2,2-trifluoroethoxy)phenyl]-1,7-dioxa-2-azaspiro[4.4]non-2-ene (Compound No. 29);
3-[3-Isopropoxy-4-(2,2,2-trifluoroethoxy)phenyl]-1,7-dioxa-2-azaspiro[4.4]non-2-ene (Compound No. 30);
3-[3-(Cyclopropylmethoxy)-4-(2,2,2-trifluoroethoxy)phenyl]-1,7-dioxa-2-azaspiro[4.4]non-2-ene (Compound No. 31);
3-[3-(2,3-Dihydro-1H-inden-2-yloxy)-4-(2,2,2-trifluoroethoxy)phenyl]-1,7-dioxa-2-azaspiro[4.4]non-2-ene (Compound No. 32);
5-(1,7-Dioxa-2-azaspiro[4.4]non-2-en-3-yl)-2-(2,2,2-trifluoroethoxy)phenol (Compound No. 33);
3-[3-Methoxy-4-(2,2,2-trifluoroethoxy)phenyl]-1,7-dioxa-2-azaspiro[4.4]non-2-ene (Compound No. 34);
3-[3-Ethoxy-4-(2,2,2-trifluoroethoxy)phenyl]-1,7-dioxa-2-azaspiro[4.4]non-2-ene (Compound No. 35);
3-[3-Butoxy-4-(2,2,2-trifluoroethoxy)phenyl]-1,7-dioxa-2-azaspiro[4.4]non-2-ene (Compound No. 36);
3-[3-(Cyclohexylmethoxy)-4-(2,2,2-trifluoroethoxy)phenyl]-1,7-dioxa-2-azaspiro[4.4]non-2-ene (Compound No. 37);
3-{[2-(Difluoromethoxy)-5-(1,7-dioxa-2-azaspiro[4.4]non-2-en-3-yl)phenoxy]methyl}benzonitrile (Compound No. 38);
2-{2-[2-(difluoromethoxy)-5-(1,7-dioxa-2-azaspiro[4.4]non-2-en-3-yl)phenoxy]ethyl}-1H-isoindole-1,3(2H)-dione (Compound No. 39);
3-[3-(Cyclohexyloxy)-4-(2,2,2-trifluoroethoxy)phenyl]-1,7-dioxa-2-azaspiro[4.4]non-2-ene (Compound No. 40);
Ethyl [5-(1,7-dioxa-2-azaspiro[4.4]non-2-en-3-yl)-2-(2,2,2-trifluoroethoxy)phenoxy]acetate (Compound No. 41);
3-[3-(Cyclohexylmethoxy)-4-(difluoromethoxy)phenyl]-1,7-dioxa-2-azaspiro[4.4]non-2-ene (Compound No. 42);
Tert-butyl [2-(difluoromethoxy)-5-(1,7-dioxa-2-azaspiro[4.4]non-2-en-3-yl)phenoxy]acetate (Compound No. 43);
N-cyclopropyl-2-[5-(1,7-dioxa-2-azaspiro[4.4]non-2-en-3-yl)-2-(2,2,2-trifluoroethoxy)phenoxy]acetamide (Compound No. 44);
2-(Cyclopentyloxy)-4-[(5R or 5S)-1,7-dioxa-2-azaspiro[4.4]non-2-en-3-yl]phenol (Compound No. 45);
2-(Cyclopentyloxy)-4-[(5S or 5R)-1,7-dioxa-2-azaspiro[4.4]non-2-en-3-yl]phenol (Compound No. 46);
N-benzyl-2-[5-(1,7-dioxa-2-azaspiro[4.4]non-2-en-3-yl)-2-(2,2,2-trifluoroethoxy)phenoxy]acetamide (Compound No. 47);
N-Cyclopentyl-2-[5-(1,7-dioxa-2-azaspiro[4.4]non-2-en-3-yl)-2-(2,2,2-trifluoroethoxy)phenoxy]acetamide (Compound No. 48);
Tert-butyl 4-[2-(difluoromethoxy)-5-(1,7-dioxa-2-azaspiro[4.4]non-2-en-3-yl)phenoxy]piperidine-1-carboxylate (Compound No. 49);
Hydrochloride salt of 3-[4-(difluoromethoxy)-3-(Piperidin-4-yloxy)phenyl]-1,7-dioxa-2-azaspiro[4.4]non-2-ene (Compound No. 50);
3-{3-[(1-Acetylpiperidin-4-yl)oxy]-4-(difluoromethoxy)phenyl}-1,7-dioxa-2-azaspiro[4.4]non-2-ene (Compound No. 51);
Tert-butyl (3S)-3-[2-(difluoromethoxy)-5-(1,7-dioxa-2-azaspiro[4.4]non-2-en-3-yl)phenoxy]pyrrolidine-1-carboxylate (Compound No. 52);
Tert-butyl (3R)-3-[2-(difluoromethoxy)-5-(1,7-dioxa-2-azaspiro[4.4]non-2-en-3-yl)phenoxy]pyrrolidine-1-carboxylate (Compound No. 53);
Tert-butyl 3-[2-(difluoromethoxy)-5-(1,7-dioxa-2-azaspiro[4.4]non-2-en-3-yl)phenoxy]piperidine-1-carboxylate (Compound No. 54);
Tert-butyl (2S)-2-{[2-(difluoromethoxy)-5-(1,7-dioxa-2-azaspiro[4.4]non-2-en-3-yl)phenoxy]methyl}pyrrolidine-1-carboxylate (Compound No. 55);
(5R or 5S)-3-[3-(cyclopentyloxy)-4-(difluoromethoxy)phenyl]-1,7-dioxa-2-azaspiro[4.4]non-2-ene (Compound No. 56);
(5S or 5R)-3-(3-isopropoxy-4-methoxyphenyl)-1,7-dioxa-2-azaspiro[4.4]non-2-ene (Compound No. 57);
(5S or 5R)-3-[3-(Cyclopropylmethoxy)-4-methoxyphenyl]-1,7-dioxa-2-azaspiro[4.4]non-2-ene (Compound No. 58);
2-(Cyclopropylmethoxy)-4-[(5S or 5R)-1,7-dioxa-2-azaspiro[4.4]non-2-en-3-yl]phenol (Compound No. 59);
4-[(5S or 5R)-1,7-Dioxa-2-azaspiro[4.4]non-2-en-3-yl]-2-isopropoxyphenol (Compound No. 60);
(5S or 5R)-3-[3-(cyclopentyloxy)-4-(difluoromethoxy)phenyl]-1,7-dioxa-2-azaspiro[4.4]non-2-ene (Compound No. 61);
(5S or 5R)-3-[3-(Cyclopropylmethoxy)-4-(difluoromethoxy)phenyl]-1,7-dioxa-2-azaspiro[4.4]non-2-ene (Compound No. 62);
(5S or 5R)-3-[4-(difluoromethoxy)-3-isopropoxyphenyl]-1,7-dioxa-2-azaspiro[4.4]non-2-ene (Compound No. 63);
(5R or 5S)-3-[4-(difluoromethoxy)-3-isopropoxyphenyl]-1,7-dioxa-2-azaspiro[4.4]non-2-ene (Compound No. 64);
2-(Cyclopropylmethoxy)-4-[(5R or 5S)-1,7-dioxa-2-azaspiro[4.4]non-2-en-3-yl]phenol (Compound No. 65);
4-[(5R or 5S)-1,7-Dioxa-2-azaspiro[4.4]non-2-en-3-yl]-2-isopropoxyphenol (Compound No. 66);
(5R or 5S)-3-[3-(Cyclopropylmethoxy)-4-(difluoromethoxy)phenyl]-1,7-dioxa-2-azaspiro[4.4]non-2-ene (Compound No. 67);
(5R or 5S)-3-[4-(difluoromethoxy)-3-isopropoxyphenyl]-1,7-dioxa-2-azaspiro[4.4]non-2-ene (Compound No. 68);
Hydrochloride salt of 3-{4-(difluoromethoxy)-3-[(3S)-pyrrolidin-3-yloxy]phenyl}-1,7-dioxa-2-azaspiro[4.4]non-2-ene (Compound No. 69);
Hydrochloride salt of 3-{4-(difluoromethoxy)-3-[(2S)-pyrrolidin-2-ylmethoxy]phenyl}-1,7-dioxa-2-azaspiro[4.4]non-2-ene (Compound No. 70);
Hydrochloride salt of 3-{4-(difluoromethoxy)-3-[(2R)-pyrrolidin-2-ylmethoxy]phenyl}-1,7-dioxa-2-azaspiro[4.4]non-2-ene (Compound No. 71);
3-[4-(Difluoromethoxy)-3-{[(2R)-1-propionylpyrrolidin-2-yl]methoxy}phenyl]-1,7-dioxa-2-azaspiro[4.4]non-2-ene (Compound No. 72);
3-[3-{[(2S)-1-acetylpyrrolidin-2-yl]methoxy}-4-(difluoromethoxy)phenyl]-1,7-dioxa-2-azaspiro[4.4]non-2-ene (Compound No. 73);
3-[3-{[(3S)-1-benzoylpyrrolidin-3-yl]oxy}-4-(difluoromethoxy)phenyl]-1,7-dioxa-2-azaspiro[4.4]non-2-ene (Compound No. 74);
3-[4-(Difluoromethoxy)-3-{[(3S)-1-propionylpyrrolidin-3-yl]oxy}phenyl]-1,7-dioxa-2-azaspiro[4.4]non-2-ene (Compound No. 75);
(5S or 5R)-3-[3-(Benzyloxy)-4-(difluoromethoxy)phenyl]-1,7-dioxa-2-azaspiro[4.4]non-2-ene (Compound No. 76);
2-(Benzyloxy)-4-[(5S or 5R)-1,7-dioxa-2-azaspiro[4.4]non-2-en-3-yl]phenol (Compound No. 77);
(5S or 5R)-3-[3-(Benzyloxy)-4-methoxyphenyl]-1,7-dioxa-2-azaspiro[4.4]non-2-ene (Compound No. 78);
3-{4-(Difluoromethoxy)-3-[(1-propionylpiperidin-4-yl)oxy]phenyl}-1,7-dioxa-2-azaspiro[4.4]non-2-ene (Compound No. 79);
3-[4-(Difluoromethoxy)-3-{[1-(4-fluorobenzoyl)piperidin-4-yl]oxy}phenyl]-1,7-dioxa-2-azaspiro[4.4]non-2-ene (Compound No. 80);
3-[3-{[1-(Cyclopropylcarbonyl)piperidin-4-yl]oxy}-4-(difluoromethoxy)phenyl]-1,7-dioxa-2-azaspiro[4.4]non-2-ene (Compound No. 81);
3-[3-{[1-(Cyclopentylcarbonyl)piperidin-4-yl]oxy}-4-(difluoromethoxy)phenyl]-1,7-dioxa-2-azaspiro[4.4]non-2-ene (Compound No. 82);
3-[4-(Difluoromethoxy)-3-({1-[(trifluoromethyl)sulfonyl]piperidin-4-yl}oxy)phenyl]-1,7-dioxa-2-azaspiro[4.4]non-2-ene (Compound No. 83);
3-{3-[(1-Acetylpiperidin-3-yl)oxy]-4-(difluoromethoxy)phenyl}-1,7-dioxa-2-azaspiro[4.4]non-2-ene (Compound No. 84);
3-{4-(Difluoromethoxy)-3-[(1-propionylpiperidin-3-yl)oxy]phenyl}-1,7-dioxa-2-azaspiro[4.4]non-2-ene (Compound No. 85);
3-[4-(Difluoromethoxy)-3-[1-(4-fluorobenzoyl)piperidin-3 oxy]phenyl]-1,7-dioxa-2-azaspiro[4.4]non-2-ene (Compound No. 86);
3-[3-{[1-(Cyclopropylcarbonyl)piperidin-3-yl]oxy}-4-(difluoromethoxy)phenyl]-1,7-dioxa-2-azaspiro[4.4]non-2-ene (Compound No. 87);
3-[3-{[1-(Cyclopentylcarbonyl)piperidin-3-yl]oxy}-4-(difluoromethoxy)phenyl]-1,7-dioxa-2-azaspiro[4.4]non-2-ene (Compound No. 88);
3-[4-(Difluoromethoxy)-3-{[1-(ethylsulfonyl)piperidin-3-yl]oxy}phenyl]-1,7-dioxa-2-azaspiro[4.4]non-2-ene (Compound No. 89);
3-[3-(Benzyloxy)-4-(2,2,2-trifluoroethoxy)phenyl]-1,7-dioxa-2-azaspiro[4.4]non-2-ene (Compound No. 90);
2-(Difluoromethoxy)-5-[(5S or 5R)-1,7-dioxa-2-azaspiro[4.4]non-2-en-3-yl]phenol (Compound No. 91);
5-[(5R or 5S)-1,7-Dioxa-2-azaspiro[4.4]non-2-en-3-yl]-2-methoxyphenol (Compound No. 92);
3-[3-{[(3S)-1-acetylpyrrolidin-3-yl]oxy}-4-(difluoromethoxy)phenyl]-1,7-dioxa-2-azaspiro[4.4]non-2-ene (Compound No. 93);
Hydrochloride salt of 3-[4-(Difluoromethoxy)-3-(piperidin-3-yloxy)phenyl]-1,7-dioxa-2-azaspiro[4.4]non-2-ene (Compound No. 94);
3-[4-(Difluoromethoxy)-3-{[1-(phenylcarbonyl)piperidin-4-yl]oxy}phenyl]-1,7-dioxa-2-azaspiro[4.4]non-2-ene (Compound No. 95);
3-[4-(Difluoromethoxy)-3-{[1-(morpholin-4-ylcarbonyl)piperidin-4-yl]oxy}phenyl]-1,7-dioxa-2-azaspiro[4.4]non-2-ene (Compound No. 96);
3-[4-(Difluoromethoxy)-3-{[1-(phenylcarbonyl)piperidin-3-yl]oxy}phenyl]-1,7-dioxa-2-azaspiro[4.4]non-2-ene (Compound No. 97);
3-[4-(Difluoromethoxy)-3-{[1-(morpholin-4-ylcarbonyl)piperidin-3-yl]oxy}phenyl]-1,7-dioxa-2-azaspiro[4.4]non-2-ene (Compound No. 98);
3-[4-(Difluoromethoxy)-3-({1-[(trifluoromethyl)sulfonyl]piperidin-3-yl}oxy)phenyl]-1,7-dioxa-2-azaspiro[4.4]non-2-ene (Compound No. 99);
3-[4-(Difluoromethoxy)-3-{[(2R)-1-(phenylcarbonyl)pyrrolidin-2-yl]methoxy}phenyl]-1,7-dioxa-2-azaspiro[4.4]non-2-ene (Compound No. 100);
3-[3-{[(2R)-1-acetylpyrrolidin-2-yl]methoxy}-4-(difluoromethoxy)phenyl]-1,7-dioxa-2-azaspiro[4.4]non-2-ene (Compound No. 101);
3-[4-(Difluoromethoxy)-3-{[(2R)-1-propanoylpyrrolidin-2-yl]methoxy}phenyl]-1,7-dioxa-2-azaspiro[4.4]non-2-ene (Compound No. 102);
3-[3-{[(2R)-1-(cyclopropylcarbonyl)pyrrolidin-2-yl]methoxy}-4-(difluoromethoxy)phenyl]-1,7-dioxa-2-azaspiro[4.4]non-2-ene (Compound No. 103);
3-[3-{[(3S)-1-(cyclopropylcarbonyl)pyrrolidin-3-yl]oxy}-4-(difluoromethoxy)phenyl]-1,7-dioxa-2-azaspiro[4.4]non-2-ene (Compound No. 104);
3-[3-{[(3S)-1-(cyclopentylcarbonyl)pyrrolidin-3-yl]oxy}-4-(difluoromethoxy)phenyl]-1,7-dioxa-2-azaspiro[4.4]non-2-ene (Compound No. 105);
3-[4-(Difluoromethoxy)-3-({(3R)-1-[(4-fluorophenyl)carbonyl]pyrrolidin-3-yl}oxy)phenyl]-1,7-dioxa-2-azaspiro[4.4]non-2-ene (Compound No. 106);
{3-[4-(Difluoromethoxy)-3-methoxyphenyl]-4,5-dihydroisoxazole-5,5-diyl}dimethanol (Compound No. 107);
3-[4-(Difluoromethoxy)-3-methoxyphenyl]-1-oxa-7-thia-2-azaspiro[4.4]non-2-ene (Compound No. 108);
3-[4-(Difluoromethoxy)-3-methoxyphenyl]-1-oxa-7-thia-2-azaspiro[4.4]non-2-ene 7-oxide (Compound No. 109);
7-[4-(Difluoromethoxy)-3-methoxyphenyl]-5-oxa-2-thia-6-azaspiro[3.4]oct-6-ene (Compound No. 110);
3-[4-(Difluoromethoxy)-3-methoxyphenyl]-1,8-dioxa-2-azaspiro[4.5]dec-2-ene (Compound No. 111);
3-[4-(Difluoromethoxy)-3-phenoxyphenyl]-1,7-dioxa-2-azaspiro[4.4]non-2-ene (Compound No. 112);
7-[4-(Difluoromethoxy)-3-methoxyphenyl]-5-oxa-2-thia-6-azaspiro[3,4]oct-6-ene 2-oxide (Compound No. 113);
3-[4-(Difluoromethoxy)-3-methoxyphenyl]-1-oxa-7-thia-2-azaspiro[4.4]non-2-ene 7,7-dioxide (Compound No. 114);
7-[4-(Difluoromethoxy)-3-methoxyphenyl]-5-oxa-6-azaspiro[3.4]oct-6-ene (Compound No. 115);
3-[4-(Difluoromethoxy)-3-methoxyphenyl]-1-oxa-2-azaspiro[4.4]non-2-ene (Compound No. 116);
3-[4-(Difluoromethoxy)-3-methoxyphenyl]-1-oxa-2-azaspiro[4,5]dec-2-ene (Compound No. 117);
3-[4-(Difluoromethoxy)-3-methoxyphenyl]-1,9,12-trioxa-2-azadispiro[4.2.4.2]tetradec-2-ene (Compound No. 118);
3-[4-(Difluoromethoxy)-3-methoxyphenyl]-1-oxa-2-azaspiro[4.5]dec-2-en-8-one (Compound No. 119);
7-[4-(Difluoromethoxy)-3-methoxyphenyl]-5-oxa-2-thia-6-azaspiro[3.4]oct-6-ene 2,2-dioxide (Compound No. 120);
3-[4-(Difluoromethoxy)-3-methoxyphenyl]-1-oxa-2-azaspiro[4.5]dec-2-en-8-one oxime (Compound No. 121);
3-[4-(Difluoromethoxy)-3-methoxyphenyl]-1-oxa-2-azaspiro[4.5]dec-2-en-8-ol (Compound No. 122);
7-[4-(Difluoromethoxy)-3-methoxyphenyl]-2,5-dioxa-6-azaspiro[3.4]oct-6-ene (Compound No. 123);
Hydrochloride salt of 4-[2-(difluoromethoxy)-5-(1,7-dioxa-2-azaspiro[4.4]non-2-en-3-yl)phenoxy]aniline (Compound No. 124);
tert-Butyl {4-[2-(difluoromethoxy)-5-(1,7-dioxa-2-azaspiro[4.4]non-2-en-3-yl)phenoxy]phenyl}carbamate (Compound No. 125);
(5R or 5S)-3-[3-(benzyloxy)-4-methoxyphenyl]-1,7-dioxa-2-azaspiro[4.4]non-2-ene (Compound No. 126);
2-(Benzyloxy)-4-[(5R or 5S)-1,7-dioxa-2-azaspiro[4.4]non-2-en-3-yl]phenol (Compound No. 127);
(5R or 5S)-3-[3-(Benzyloxy)-4-(difluoromethoxy)phenyl]-1,7-dioxa-2-azaspiro[4.4]non-2-ene (Compound No. 128);
3-[2-(Difluoromethoxy)-5-(1,7-dioxa-2-azaspiro[4.4]non-2-en-3-yl)phenoxy]cyclopentanol (Compound No. 129);
2-(Difluoromethoxy)-5-[(5R or 5S)-1,7-dioxa-2-azaspiro[4.4]non-2-en-3-yl]phenol (Compound No. 130);
3-[4-(Difluoromethoxy)-3-(4-fluorophenoxy)phenyl]-1,7-dioxa-2-azaspiro[4.4]non-2-ene (Compound No. 131);
3-[3-(4-Chlorophenoxy)-4-(difluoromethoxy)phenyl]-1,7-dioxa-2-azaspiro[4.4]non-2-ene (Compound No. 132);
3-{4-(Difluoromethoxy)-3-[4-(trifluoromethoxy)phenoxy]phenyl}-1,7-dioxa-2-azaspiro[4.4]non-2-ene (Compound No. 133);
3-{4-(Difluoromethoxy)-3-[4-(trifluoromethyl)phenoxy]phenyl}-1,7-dioxa-2-azaspiro[4.4]non-2-ene (Compound No. 134);
N-{4-[2-(Difluoromethoxy)-5-(1,7-dioxa-2-azaspiro[4.4]non-2-en-3-yl)phenoxy]phenyl}acetamide (Compound No. 135);
N-{4-[2-(Difluoromethoxy)-5-(1,7-dioxa-2-azaspiro[4.4]non-2-en-3-yl)phenoxy]phenyl}methane sulfonamide (Compound No. 136);
3-[4-(Difluoromethoxy)-3-(pyridin-4-yloxy)phenyl]-1,7-dioxa-2-azaspiro[4.4]non-2-ene (Compound No. 137);
pharmaceutically acceptable salts, pharmaceutically acceptable solvates, enantiomers, diastereomers, polymorphs or N-oxides thereof.

3. A pharmaceutical composition comprising a therapeutically effective amount of a compound as defined in claim 1 or 2 together with one or more of pharmaceutically acceptable carriers, excipients or diluents.

4. A pharmaceutical composition comprising a therapeutically effective amount of a compound of claim 1 or 2 and at least one other active ingredient selected from corticosteroids, β2-agonists, muscarinic receptor antagonists, anticholinergics, antiallergic agents, PAF antagonists, EGFR kinase inhibitors, p38 MAP Kinase inhibitors, additional PDE-IV inhibitors, kinase inhibitors, dopamine receptor antagonists, histamines, antitussives, leukotriene antagonists, 5-lipoxygenase inhibitors, chemokine inhibitors or combinations thereof.

5. A method for treating, preventing, inhibiting or suppressing an inflammatory condition or disease or CNS diseases, in a patient, comprising administering to the said patient a therapeutically effective amount of a compound of claim 1 or 2.

6. A method for treating, preventing, inhibiting or suppressing an inflammatory condition or disease or CNS diseases, in a patient, comprising administering to the said patient a therapeutically effective amount of a pharmaceutical composition of claim 3 or 4.

7. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a compound of claim 1 or 2.

8. A method for the treatment, prevention, inhibition or suppression of CNS diseases, AIDS, asthma, arthritis, bronchitis, chronic obstructive pulmonary disease (COPD), psoriasis, allergic rhinitis, shock, atopic dermatitis, Crohn's disease, adult respiratory distress syndrome (ARDS), eosinophilic granuloma, allergic conjunctivitis, osteoarthritis, ulcerative colitis and other inflammatory diseases in a patient comprising administering to said patient a therapeutically effective amount of a pharmaceutical composition of claim 3 or 4.

9. A method according to claim 5, 6, 7 or 8 wherein, the disease or disorder is mediated through phosphodiesterase type 4 or 7.

10. A method for the preparation of a compound of Formula II, and its pharmaceutically acceptable salts, pharmaceutically acceptable solvates, enantiomers, diastereomers, polymorphs or N-oxides, wherein the method comprises,
a. deprotecting a compound of Formula Ia 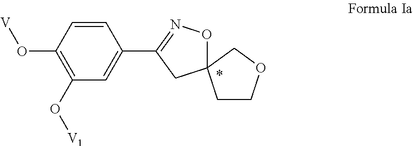
to give a compound of Formula II 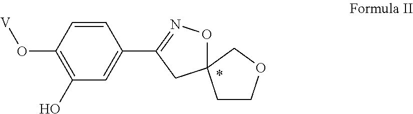
wherein * refers to chiral centre (racemic or R or S isomer),
V is alkyl,
V₁ is cycloalkyl.

11. A method for the preparation of a compound of Formula IV, and its pharmaceutically acceptable salts, pharmaceutically acceptable solvates, enantiomers, diastereomers, polymorphs or N-oxides, wherein the method comprises,
a. deprotecting a compound of Formula Ia 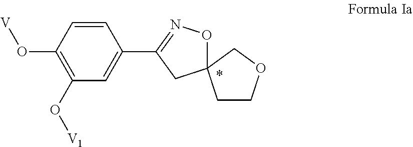
to give a compound of Formula II 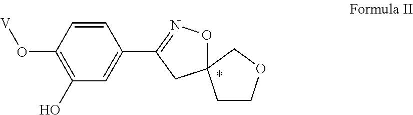
b. reacting a compound of Formula II with a compound of Formula III
Ryy-hal Formula III
to give a compound of Formula IV 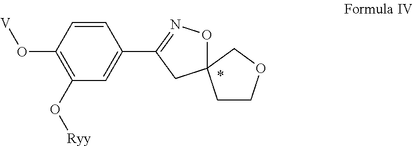
wherein
* refers to chiral centre (racemic or R or S isomer),
V is alkyl,
V₁ is cycloalkyl,
hal is Br, C or I,
Ryy is alkyl, aryl, cycloalkyl, alkaryl, heteroaryl, heterocyclyl, heteroarylalkyl, heterocyclylalkyl, —(CH₂)_{g1}COOR₃, —(CH₂)ₘCOR₃ or —(CH₂)_{g}C(═O)NRₓR_{y}. wherein R₃, g, m, Rₓ, R_{y} and g₁ are the same as defined in claim 1.

12. A method for the preparation of a compound of Formula V, and its pharmaceutically acceptable salts, pharmaceutically acceptable solvates, enantiomers, diastereomers, polymorphs or N-oxides, wherein the method comprises,
a. deprotecting a compound of Formula Ia 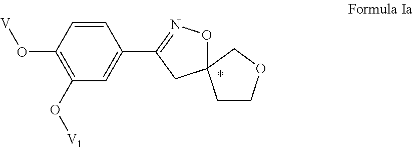
to give a compound of Formula II 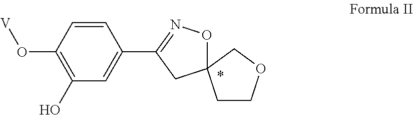
b. reacting a compound of Formula II with a compound of Formula III
Ryy-hal Formula III
to give a compound of Formula IV 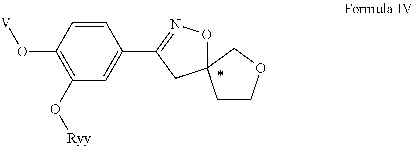
c. deprotecting a compound of Formula IV to give a compound of Formula V 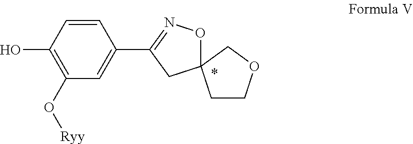
wherein
* refers to chiral centre (racemic or R or S isomer),
V is alkyl,
V₁ is cycloalkyl,
hal is Br, Cl or I,
Ryy is alkyl, aryl, cycloalkyl, alkaryl heteroaryl, heterocyclyl, heteroarylalkyl, heterocyclylalkyl, —(CH₂)_{g1}COOR₃, —(CH₂)ₘCOR₃ or —(CH₂)_{g}C(═O)NRₓR_{y},
wherein R₃, g, m, Rₓ, R_{y} and g₁ are the same as defined in claim 1.

13. A method for the preparation of a compound of Formula VI, and its pharmaceutically acceptable salts, pharmaceutically acceptable solvates, enantiomers, diastereomers, polymorphs or N-oxides, wherein the method comprises,
a. deprotecting a compound of Formula Ia 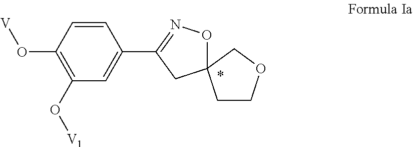
to give a compound of Formula II 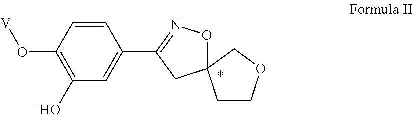
b. reacting a compound of Formula II with a compound of Formula III
R_{yy}-Hal Formula III
to give a compound of Formula IV 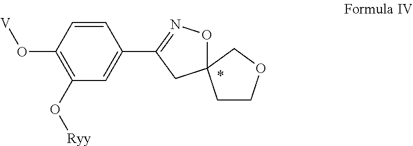
c. deprotecting a compound of Formula to give a compound of Formula V 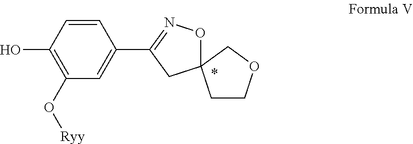
d. reacting a compound of Formula V with a compound of Formula IIIc
R_{xy}-Hal Formula IIIa
to give a compound of Formula VI 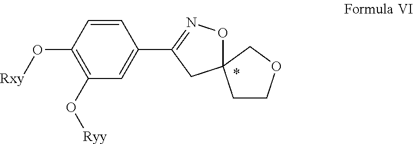
wherein
* refers to chiral centre (racemic or R or S isomer),
V is alkyl,
V₁ is cycloalkyl,
hal is Br, Cl or I,
Ryy is alkyl, aryl, cycloalkyl, alkaryl, heteroaryl, heterocyclyl, heteroarylalkyl, heterocyclylalkyl, —(CH₂)_{g1}COOR₃, —(CH₂)ₘCOR₃ or —(CH₂)_{g}C(O)NRₓR_{y},
wherein R₃, g, m, Rₓ, R_{y} and g₁ are the same as defined in claim 1,
Rxy is alkyl, cycloalkyl, alkaryl, heteroaryl, heterocyclyl, heteroarylalkyl, heterocyclylalkyl.

14. A method for the preparation of a compound of Formula VII, and its pharmaceutically acceptable salts, pharmaceutically acceptable solvates, enantiomers, diastereomers, polymorphs or N-oxides, wherein the method comprises,
a. deprotecting a compound of Formula Ia 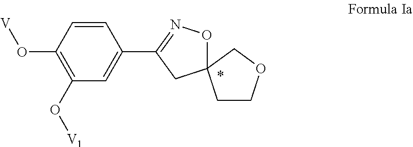
to give a compound of Formula II 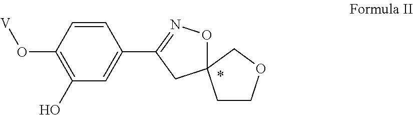
b. reacting a compound of Formula II with a compound of Formula III
Ryy-hal Formula III
to give a compound of Formula IV 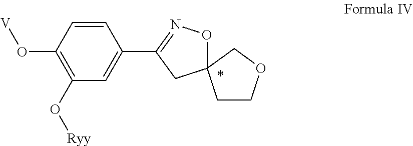
c. deprotecting a compound of Formula IV to give a compound of Formula V 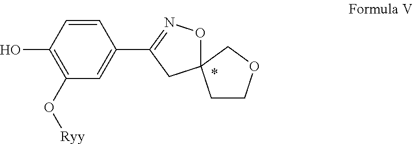
d. reacting a compound of Formula V with a compound of Formula IIIa
Rxy-hal Formula IIIa
to give a compound of Formula VI 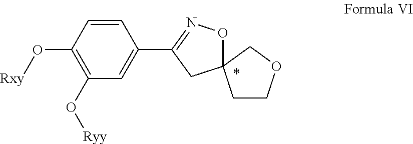
e. deprotecting a compound of Formula VI to give a compound of Formula VII 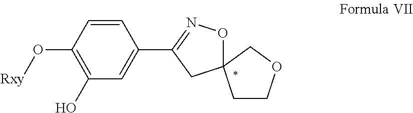
wherein
* refers to chiral centre (racemic or R or S isomer),
V is alkyl,
V₁ is cycloalkyl,
hal is Br, Cl or I,
Ryy is alkyl, aryl, cycloalkyl, alkaryl, heteroaryl, heterocyclyl, heteroarylalkyl, heterocyclylalkyl, —(CH₂)_{g1}COOR₃, —(CH₂)ₘCOR₃ or —(CH₂)_{g}C(═O)NRₓR_{y},
wherein R₃, g, m, Rₓ, R_{y} and g₁ are the same as defined in claim 1,
Rxy is alkyl, cycloalkyl, alkaryl, heteroaryl, heterocyclyl, heteroarylalkyl, heterocyclylalkyl.

15. A method for the preparation of a compound of Formula IX, and its pharmaceutically acceptable salts, pharmaceutically acceptable solvates, enantiomers, diastereomers, polymorphs or N-oxides, wherein the method comprises
a. deprotecting a compound of Formula Ia 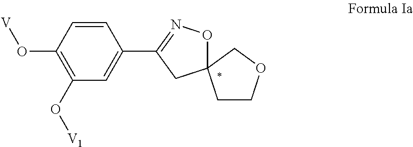
to give a compound of Formula II 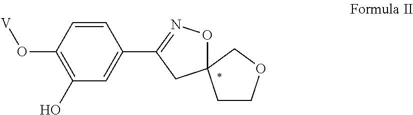
b. reacting a compound of Formula II with a compound of Formula III
Ryy-hal Formula III
to give a compound of Formula IV 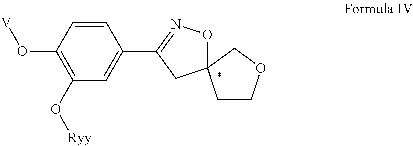
c. deprotecting a compound of Formula IV to give a compound of Formula V 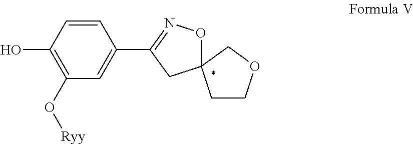
d. reacting a compound of Formula V with a compound of Formula IIIa
Rxy-hal Formula IIIa
to give a compound of Formula VI 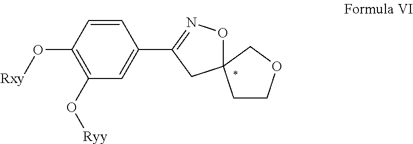
e. deprotecting a compound of Formula VI to give a compound of Formula VII 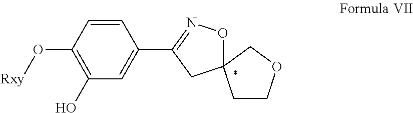
f. reacting a compound of Formula VII with a compound of Formula VIII
Rff-CO-hal Formula VIII
to give a compound of Formula IX 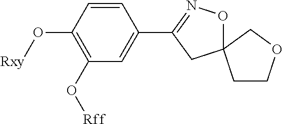
wherein * refers to chiral centre (racemic or R or S isomer),
V is alkyl,
V₁ is cycloalkyl,
hal is Br, Cl or I.
Ryy is alkyl, aryl, cycloalkyl, alkaryl, heteroaryl, heterocyclyl, heteroarylalkyl, heterocyclylalkyl, —(CH₂)_{g1}COOR₃, —(CH₂)_{g}COR₃ or —(CH₂)_{g}C(═O)NRₓR_{y},
wherein R₃, g, m, Rₓ, R_{y} and g₁ are the same as defined in claim 1,
Rxy is alkyl, cycloalkyl, alkaryl, heteroaryl, heterocyclyl, heteroarylalkyl, heterocyclylalkyl,
Rff is alkyl, cycloalkyl, alkaryl, aryl, heteroaryl, heterocyclyl, heteroarylalkyl or heterocyclylalkyl.

16. A method for the preparation of a compound of Formula XI, and its pharmaceutically acceptable salts, pharmaceutically acceptable solvates, enantiomers, diastereomers, polymorphs or N-oxides, wherein the method comprises,
a. deprotecting a compound of Formula Ia 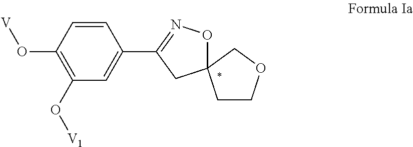
to give a compound of Formula II 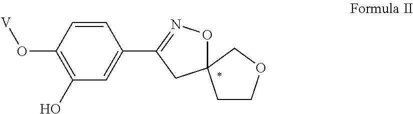
b. reacting a compound of Formula II with a compound of Formula III
Ryy-hal Formula III
to give a compound of Formula IV 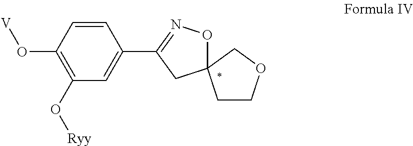
c. deprotecting a compound of Formula IV to give a compound of Formula V 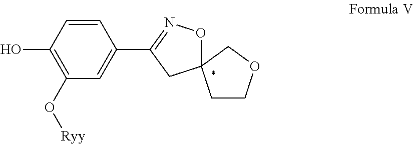
d. reacting a compound of Formula V with a compound of Formula IIIa
Rxy-hal Formula IIIa
to give a compound of Formula VI 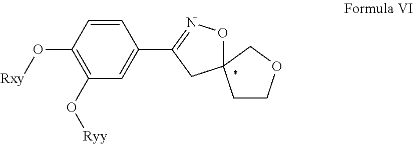
e. deprotecting a compound of Formula VI to give a compound of Formula VII 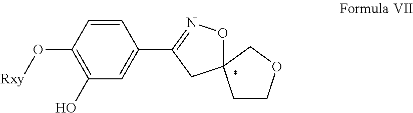
f. reacting a compound of Formula VII with a compound of Formula X
R₃y-hal Formula X
to give a compound of Formula XI 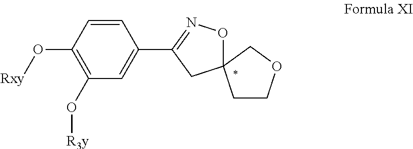
wherein
V is alkyl,
V₁ is cycloalkyl,
hal is Br, Cl or I,
Ryy is alkyl, aryl, cycloalkyl, alkaryl, heteroaryl, heterocyclyl, heteroarylalkyl, heterocyclylalkyl —(CH₂)_{g1}COOR₃, —(CH₂)ₘCOR₃ or —(CH₂)_{g}C(═O)NRₓR_{y},
Rxy is alkyl, cycloalkyl, alkaryl, heteroaryl, heterocyclyl, heteroarylalkyl, heterocyclylalkyl,
Rff is alkyl, cycloalkyl, alkaryl, aryl, heteroaryl, heterocyclyl, heteroarylalkyl or heterocyclylalkyl,
R_{3y} is —(CH₂)_{g1}C(═O)OR₃, —(CH₂)ₘCOR₃, alkyl, cycloalkyl, alkaryl, heteroaryl, heterocyclyl, heteroarylalkyl, heterocyclylalkyl or —(CH₂)_{g}C(═O)NRₓR_{y},
R₃, g, m, Rₓ, R_{y} and g₁ are the same as defined in claim 1.

17. A method for the preparation of a compound of Formula XIII, and its pharmaceutically acceptable salts, pharmaceutically acceptable solvates, enantiomers, diastereomers, polymorphs or N-oxides, wherein the method comprises
a. deprotecting a compound of Formula Ia 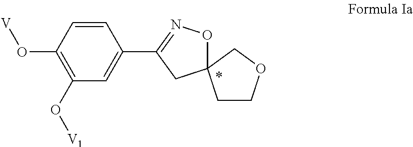
to give a compound of Formula II 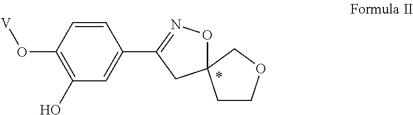
b. reacting a compound of Formula II with a compound of Formula III
Ryy-hal Formula III
to give a compound Formula IV 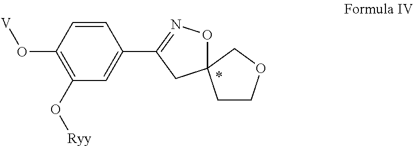
c. deprotecting a compound of Formula IV to give a compound of Formula V 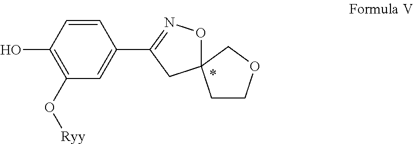
d. reacting a compound of Formula V with a compound of Formula IIIa
Rxy-hal Formula IIIa
to give a compound of Formula VI 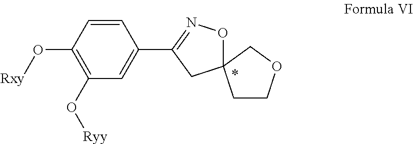
e. deprotecting a compound of Formula VI to give a compound of Formula VII 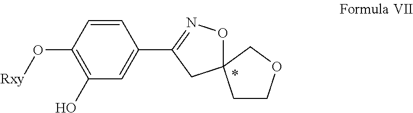
f. reacting a compound of Formula VII with a compound of Formula X
R₃y-hal Formula X
to give a compound of Formula XI 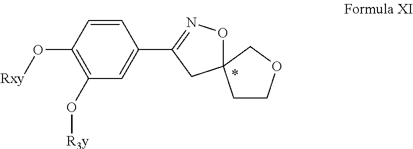
g. reacting a compound of Formula XI with a compound of Formula XII
NH₂—P Formula XII
to give a compound of Formula XIII 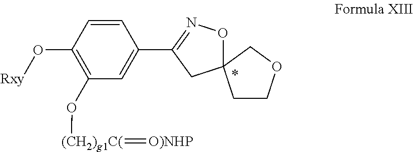
wherein * refers to chiral centre (racemic or R or S isomer),
V is alkyl,
V₁ is cycloalkyl,
hal is Br, Cl or I,
Ryy is alkyl, aryl, cycloalkyl, alkaryl, heteroaryl, heterocyclyl, heteroarylalkyl, heterocyclylalkyl, —(CH₂)_{g1}COOR₃, —(CH₂)ₘCOR³ or —(CH₂)_{g}C(═O)NRₓR_{y},
Rxy is alkyl, cycloalkyl, alkaryl, heteroaryl, heterocyclyl, heteroarylalkyl, heterocyclylalkyl,
Rff is alkyl, cycloalkyl, alkaryl, aryl, heteroaryl, heterocyclyl, heteroarylalkyl or heterocyclylalkyl,
R_{3y} is —(CH₂)_{g1}C(═O)OR₃, —(CH₂)ₘCOR₃, alkyl, cycloalkyl, alkaryl, heteroaryl, heterocyclyl, heteroarylalkyl, heterocyclylalkyl or —(CH₂)_{g}C(═O)NRₓR_{y},
P is alkyl, aralkyl, cycloalkyl, —C(═O)Oaralkyl, —C(═O)OC(CH₃)₃, —C(═O)OC(CH₃)₂CHBr₂ or —C(═O)OC(CH₃)₂CCl₃,
R₃, g, m, Rₓ, R_{y} and g₁ are the same as defined in claim 1.

18. A method for the preparation of a compound of Formula IIa, and its pharmaceutically acceptable salts, pharmaceutically acceptable solvates, enantiomers, diastereomers, polymorphs or N-oxides, wherein the method comprises
deprotecting a compound of Formula Ia 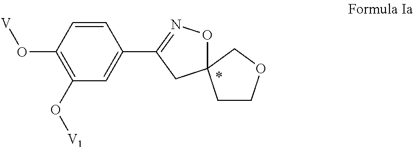
to give a compound of Formula IIa 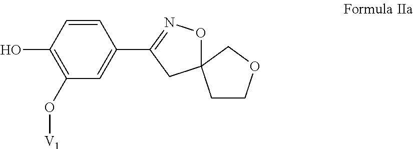
wherein
V is alkyl,
V₁ is cycloalkyl,
* refers to chiral centre (racemic or R or S isomer).

19. A method for the preparation of a compound of Formula IVa, and its pharmaceutically acceptable salts, pharmaceutically acceptable solvates, enantiomers, diastereomers, polymorphs or N-oxides, wherein the method comprises
a. deprotecting a compound of Formula Ia 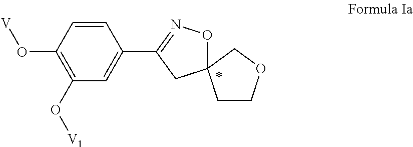
to give a compound of Formula IIa 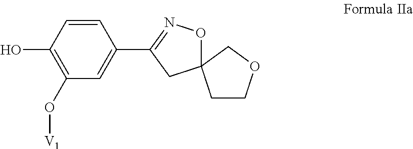
b. reacting a compound of Formula IIa with a compound of Formula III
Ryy-hal Formula III
to give a compound of Formula IVa 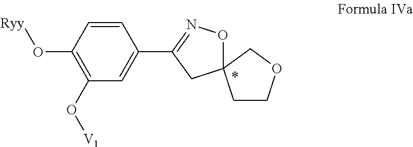
wherein
V is alkyl,
V₁ is cycloalkyl,
hal is Br, Cl or I,
Ryy is alkyl, aryl, cycloalkyl, alkaryl, heteroaryl, heterocyclyl, heteroarylalkyl or heterocyclylalkyl, —(CH₂)₂C(O)NRₓR_{y}, —(CH₂)ₘCOR₃ or —(CH₂)_{g1}C(═O)OR₃,
wherein R₃, g, m, Rₓ, R_{y} and g₁ are the same as defined in claim 1,
* refers to chiral centre (racemic or R or S isomer).

20. A method for the preparation of a compound of Formula XVII, and its pharmaceutically acceptable salts, pharmaceutically acceptable solvates, enantiomers, diastereomers, polymorphs or N-oxides wherein the method comprises:
a. reacting a compound of Formula XIV with a compound of Formula XV, 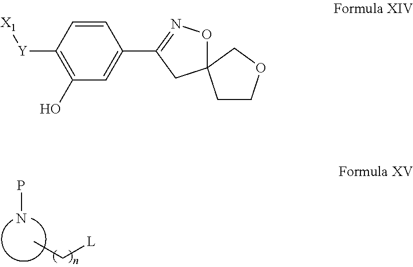
to give a compound of Formula XVI 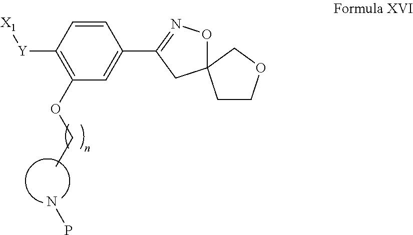
b. deprotecting a compound of Formula XVI to give a compound of Formula XVII 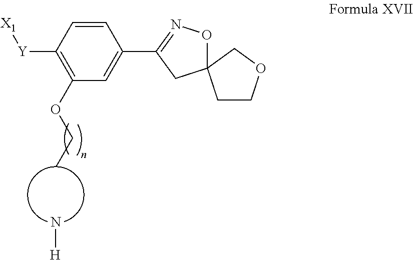
wherein Y and X are the same as defined in claim 1,
P is alkyl, aralkyl, cycloalkyl, —C(═O)Oaralkyl, —C(═O)OC(CH₃)₃, —C(═O)OC(CH₃)₂CHBr₂ or —C(═O)OC(CH₃)₂CCl₃,
L is a leaving group selected from hal (Br, Cl or I), —Omesyl, —Otosyl or —Otriflyl,
n is an integer from 0-2.

21. A method for the preparation of a compound of Formula XIX, and its pharmaceutically acceptable salts, pharmaceutically acceptable solvates, enantiomers, diastereomers, polymorphs or N-oxides wherein the method comprises
a. reacting a compound of Formula XIV with a compound of Formula XV 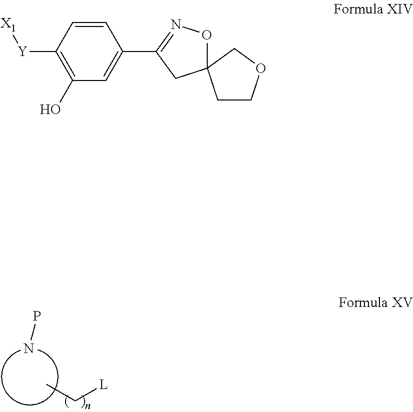
to give a compound of Formula XVI 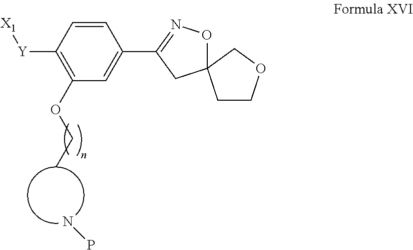
b. deprotecting a compound of Formula XVI to give a compound of Formula XVII 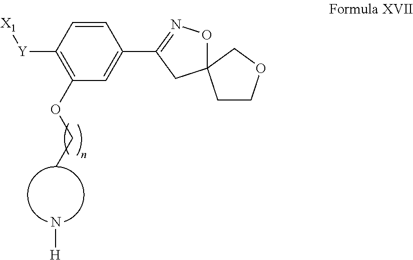
c. reacting a compound of Formula XVII with a compound of Formula XVIII
Rff-G-hal Formula XVIII
to give a compound of Formula XIX 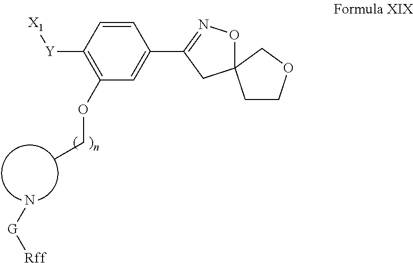
wherein Y and X₁ are the same as defined in claim 1,
P is alkyl, aralkyl, cycloalkyl, —C(═O)Oaralkyl, —C(═O)OC(CH₃)₃, —C(═O)OC(CH₃)₂CHBr₂ or —C(═O)OC(CH₃)₂CCl₃,
Rff is alkyl, cycloalkyl, alkaryl, aryl, heteroaryl, heterocyclyl, heteroarylalkyl or heterocyclylalkyl,
hal is Br, Cl or I,
is a leaving group selected from hal (Br, Cl or I), —Omesyl, —Otosyl or —Otriflyl,
n is an integer from 0-2,
G is —CO or —SO₂.

22. A method for the preparation of a compound of Formula XXIV, and its pharmaceutically acceptable salts, pharmaceutically acceptable solvates, enantiomers, diastereomers, polymorphs or N-oxides, wherein the method comprises
a. reacting a compound of Formula XX 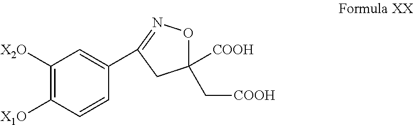
with a compound of Formula XXa
Q Formula XXa
to give a compound of Formula XXI 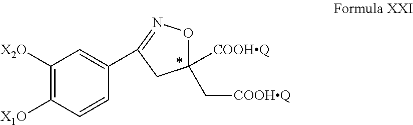
b. reacting a compound of Formula XXI with a compound of Formula P′—OH to give a compound of Formula XXII 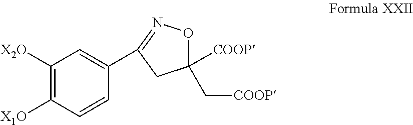
c. reducing a compound of Formula XXII to give a compound of Formula XXIII 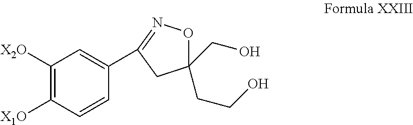
d. cyclizing a compound of Formula XXIII to give a compound of Formula XXIV 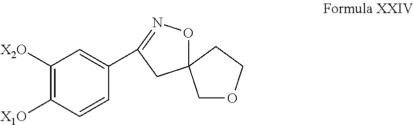
wherein
X₁ and X₂ are the same as defined in claim 1,
Q is a chiral resolving agent selected from L-Ephedrine, D-Ephedrine, (+)-Brussian, (−)-Brussian, (1S,2R) (−)-cis-1-amino-2-indanol, (1R 2S) (+)-cis-1-amino-2-indanol, (1R,2R)-(−)-1,2-diamino cyclohexane or (1S,2S)-(+)-1,2-diamino cyclohexane, α-methylbenzylamine or β-methylbenzylamine,
* refers to chiral centre (racemic or R or S isomer),
P′ is alkyl or aralkyl.

23. A method for the preparation of a compound of Formula XXV b, and its pharmaceutically acceptable salts, pharmaceutically acceptable solvates, enantiomers, diastereomers, polymorphs or N-oxides, wherein the method comprises
reacting a compound of Formula XXV with a compound of Formula XXV a 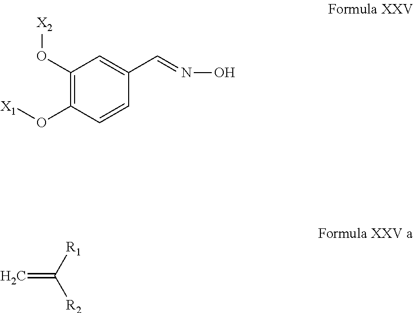
to give a compound of Formula XXV b 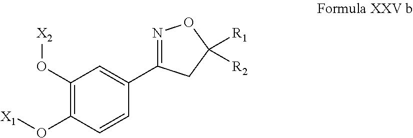
wherein
X₁, X₂, R₁ and R₂ are the same as defined in claim 1.

24. A method for the preparation of a compound of Formula XXVIII, and its pharmaceutically acceptable salts, pharmaceutically acceptable solvates, enantiomers, diastereomers, polymorphs or N-oxides, wherein the method comprises
a. mesylating a compound of Formula XXVI 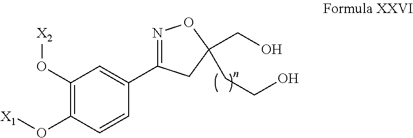
to give a compound of Formula XXVII 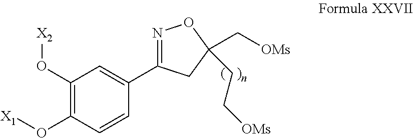
b. cyclizing a compound of Formula XXVII to give a compound of Formula XXVIII 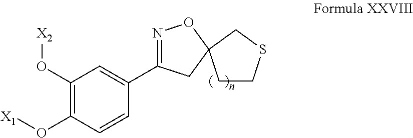
wherein
X₁ and X₂ are the same as defined in claim 1,
n is an integer from 0-2.

25. A method for the preparation of a compound of Formula XXIX, and its pharmaceutically acceptable salts, pharmaceutically acceptable solvates, enantiomers, diastereomers, polymorphs or N-oxides, wherein the method comprises
a. mesylating a compound of Formula XXVI 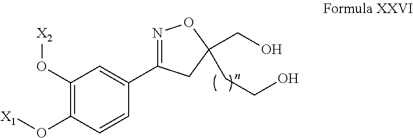
to give a compound of Formula XXVII 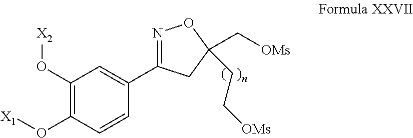
b. cyclizing a compound of Formula XXVII to give a compound of Formula XXVIII 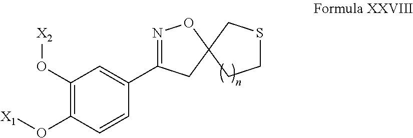
c. oxidizing a compound of Formula XXVIII to give a compound of Formula XXIX 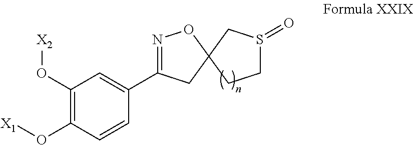
wherein
X₁ and X₂ are the same as defined in claim 1,
n is an integer from 0-2.

26. A method for the preparation of a compound of Formula XXX, and its pharmaceutically acceptable salts, pharmaceutically acceptable solvates, enantiomers, diastereomers, polymorphs or N-oxides, wherein the method comprises
a. mesylating a compound of Formula XXVI 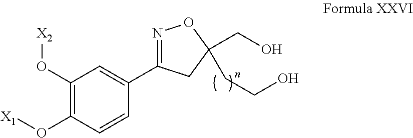
to give a compound of Formula XXVII 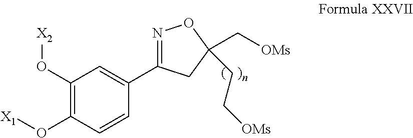
b. cyclizing a compound of Formula XXVII to give a compound of Formula XXVIII 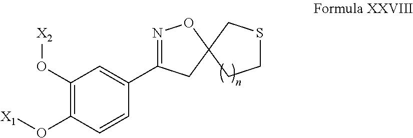
c. oxidizing a compound of Formula XXVIII to give a compound of Formulae XXX 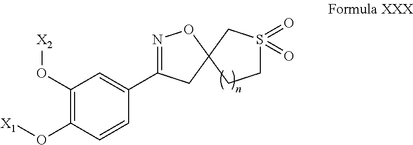
wherein
X₁ and X₂ are the same as defined in claim 1,
n is an integer from 0-2.

27. A method for the preparation of a compound of Formula XXXIV, and its pharmaceutically acceptable salts, pharmaceutically acceptable solvates, enantiomers, diastereomers, polymorphs or N-oxides, wherein the method comprises
a. reacting a compound of Formula XXV with a compound of Formula XXXI 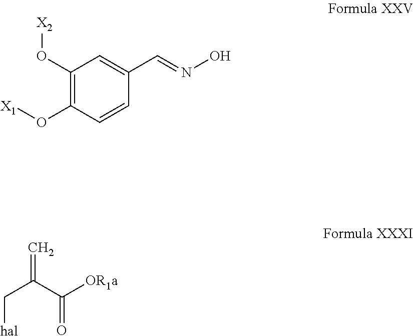
to give a compound of Formula XXXII 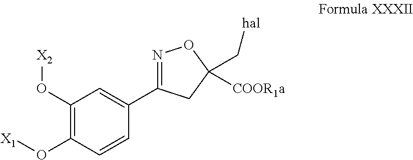
b. performing reduction of a compound of Formula XXXII to give a compound of Formula XXXIII 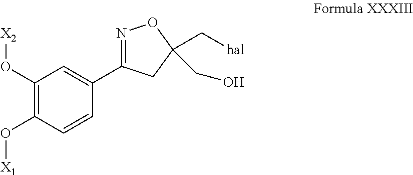
c. cyclizing a compound of Formula XXXIII to give a compound of Formula XXXIV 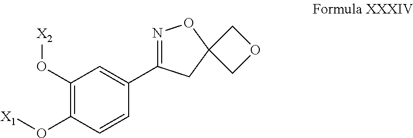
wherein X₁ and X₂ are the same as defined in claim 1,
hal is Br, Cl or I,
R₁ₐ is alkyl.

28. A method for the preparation of a compound of Formula XXXVII, and its pharmaceutically acceptable salts, pharmaceutically acceptable solvates, enantiomers, diastereomers, polymorphs or N-oxides, wherein the method comprises
a. reacting a compound of Formula XXXV with a compound of Formula XXXV a 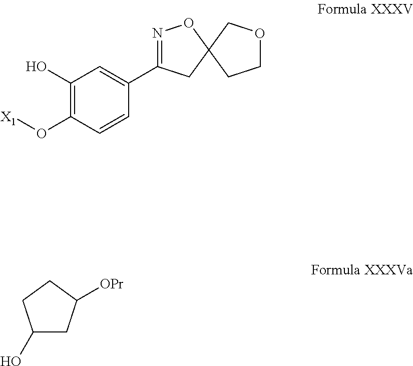
to give a compound of Formula XXXVI 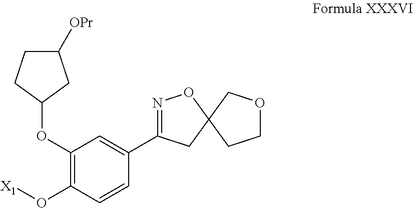
b. deprotecting a compound of Formula XXXVI to give a compound of Formula XXVII 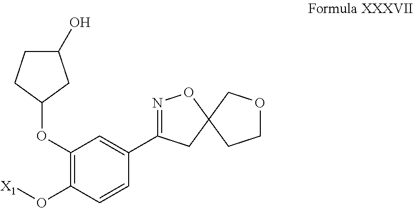
wherein
X₁ is the same as defined in claim 1,
Pr is a protecting group.

29. A method for the preparation of a compound of Formula XXXIX, and its pharmaceutically acceptable salts, pharmaceutically acceptable solvates, enantiomers, diastereomers, polymorphs or N-oxides, wherein the method comprises
reacting a compound of Formula XXXV with a compound of Formula XXXV 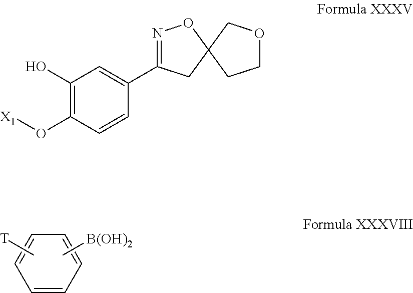
to give a compound of Formula XXXIX 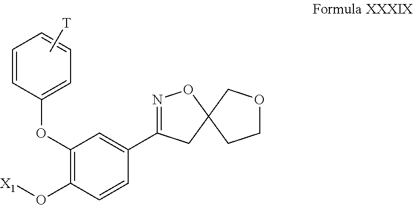
wherein
X₁ is the same as defined in claim 1,
T is halogen, alkoxy, alkyl or —NHCOOalkyl.

30. A method for the preparation of a compound of Formula XL, and its pharmaceutically acceptable salts, pharmaceutically acceptable solvates, enantiomers, diastereomers, polymorphs or N-oxides, wherein the method comprises
a. reacting a compound of Formula XXXV with a compound of Formula XXXVIII 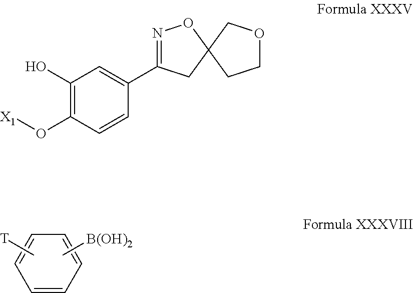
to give a compound of Formula XXXIX 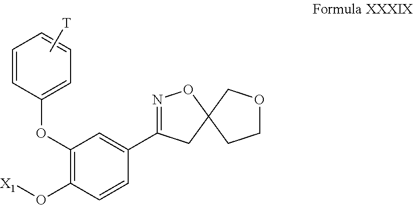
b. deprotecting a compound of Formula XXXIX (when T is —NHCOOalkyl) to give a compound of Formula XL 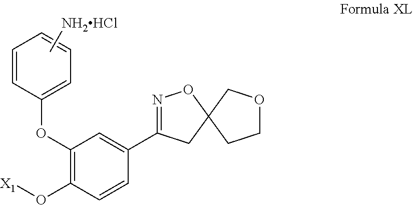
wherein
X₁ is the same as defined in claim 1,
T is halogen, alkoxy, alkyl or —NHCOOalkyl.

31. A method for the preparation of a compound of Formula XLI, and its pharmaceutically acceptable salts, pharmaceutically acceptable solvates, enantiomers, diastereomers, polymorphs or N-oxides, Wherein the method comprises
a. reacting a compound of Formula XXXV with a compound of Formula XXXVIII 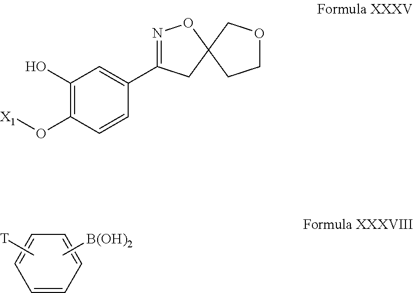
to give a compound of Formula XXXIX 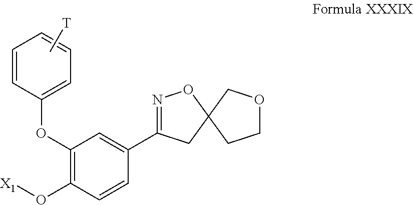
b. deprotecting a compound of Formula XXXIX (when T is —NHCOOalkyl) to give a compound of Formula XL 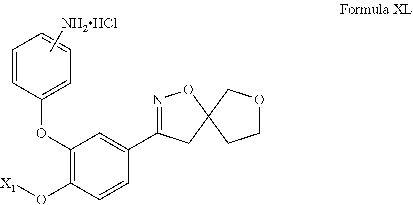
c. mesylating a compound of Formula XL to give a compound of Formula XLI 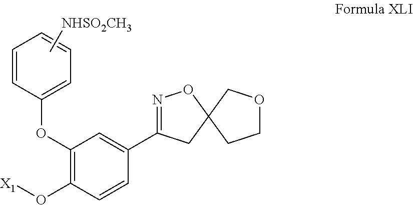
wherein
X₁ is the same as defined in claim 1,
T is halogen, alkoxy, alkyl or —NHCOOalkyl.

32. A method for the preparation of a compound of Formula XLII, and its pharmaceutically acceptable salts, pharmaceutically acceptable solvates, enantiomers, diastereomers, polymorphs or N-oxides, wherein the method comprises
a. reacting a compound of Formula XXXV with a compound of Formula XXXVIII 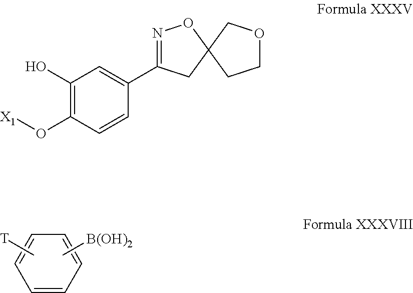
to give a compound of Formula XXXIX 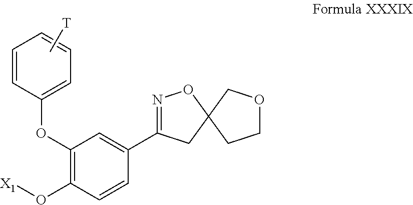
b. deprotecting a compound of Formula XXXIX (when T is —NHCOOalkyl) to give a compound of Formula XL 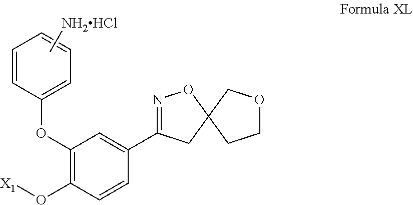
c. acylating a compound of Formula XL to give a compound of Formula XLII 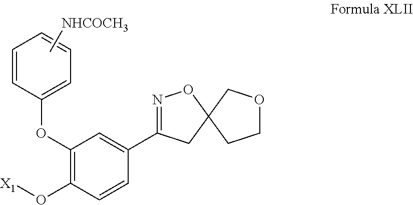
wherein
X is the same as defined in claim 1,
T is halogen, alkoxy, alkyl or —NHCOOalkyl.

33. A method for the preparation of a compound of Formula XLIV, and its pharmaceutically acceptable salts, pharmaceutically acceptable solvates, enantiomers, diastereomers, polymorphs or N-oxides, wherein the method comprises
reacting a compound of Formula XXXV with a compound of Formula XLIII 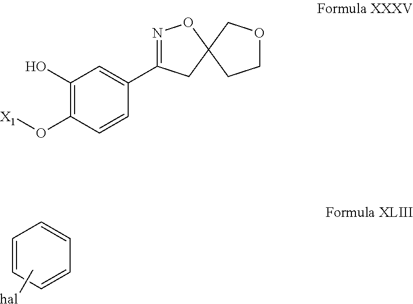
to give a compound of formula XLIV 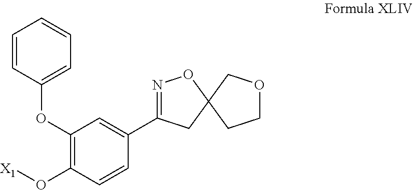
wherein
hal is Br, Cl or I,
X₁ is the same as defined in claim 1.

34. A method for the preparation of a compound of Formula XLVI, and its pharmaceutically acceptable salts, pharmaceutically acceptable solvates, enantiomers, diastereomers, polymorphs or N-oxides, wherein the method comprises
reacting a compound of Formula XXXV with a compound of Formula XLV 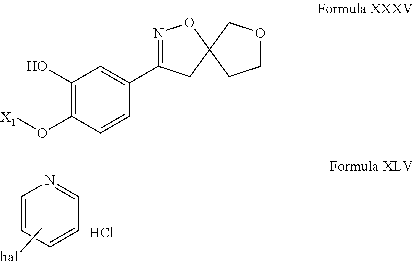
to give a compound Formula XLVI 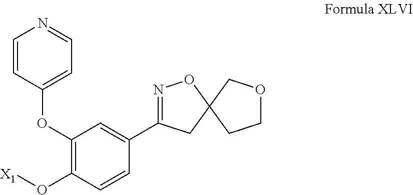
wherein
hal is Br, Cl or I,
X₁ is the same as defined in claim 1.

35. A method for the preparation of a compound of Formula XLVIII, and its pharmaceutically acceptable salts, pharmaceutically acceptable solvates, enantiomers, diastereomers, polymorphs or N-oxides, wherein the method comprises
reacting a compound of Formula XXV with a compound of Formula XLVII 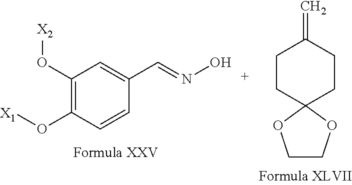
to give a compound of Formula XLVIII 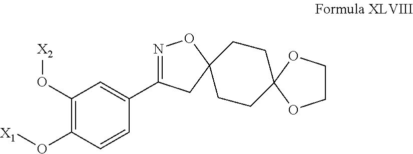
wherein
X₁ and X₂ are the same as defined in claim 1.

36. A method for the preparation of a compound of Formula XLIX, and its pharmaceutically acceptable salts, pharmaceutically acceptable solvates, enantiomers, diastereomers, polymorphs or N-oxides, wherein the method comprises
a. reacting a compound of Formula XXV with a compound of Formula XLVII 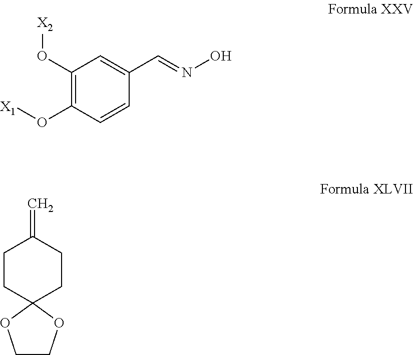
to give a compound of Formula XLVIII 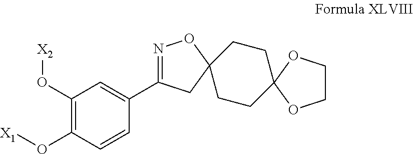
b. deprotecting a compound of Formula XLVIII to give a compound of Formula XLIX 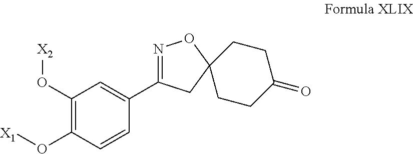
wherein
X₁ and X₂ are the same as defined in claim 1.

37. A method for the preparation of a compound of Formula L, and its pharmaceutically acceptable salts, pharmaceutically acceptable solvates, enantiomers, diastereomers, polymorphs or N-oxides, wherein the method comprises
a. reacting a compound of Formula XXV with a compound of Formula XLVII 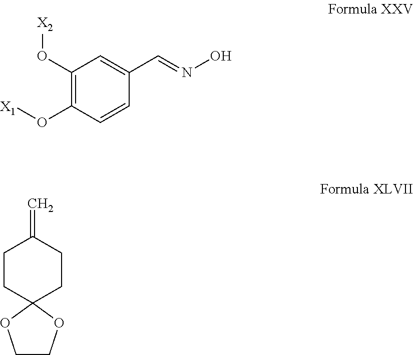
to give a compound of Formula XLVIII 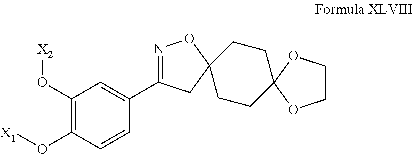
b. deprotecting a compound of Formula XLVIII to give a compound of Formula XLIX 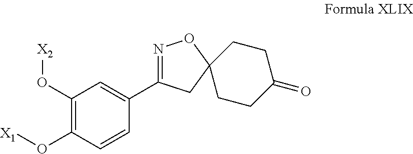
c. performing the reduction of a compound of Formula XLIX to give a compound of Formula L 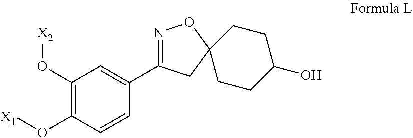
wherein
X₁ and X₂ are the same as defined in claim 1.

38. A method for the preparation of a compound of Formula LI, and its pharmaceutically acceptable salts, pharmaceutically acceptable solvates, enantiomers, diastereomers, polymorphs or N-oxides, wherein the method comprises
a. reacting a compound of Formula XXV with a compound of Formula XLVII 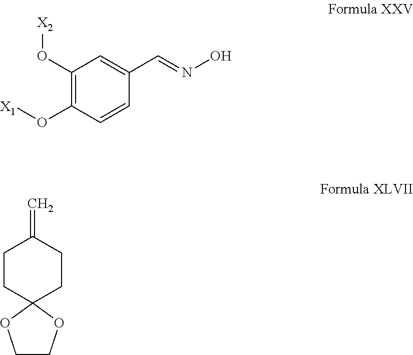
to give a compound of Formula XLVIII 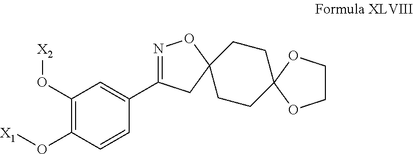
b. deprotecting a compound of Formula XLVIII to give a compound of Formula XLIX 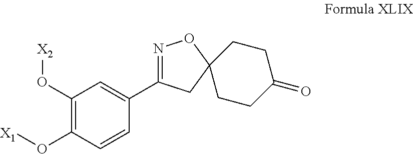
c. reacting a compound of Formula XLIX with hydroxylamine hydrochloride to give a compound of Formula LI 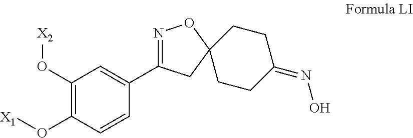
wherein
X₁ and X₂ are the same as defined in claim 1.
